(19) United States
(12) Patent Application Publication
(10) Pub. No.: US 2019/0169675 A1
(43) Pub. Date: Jun. 6, 2019

(54) PROTEOMIC ANALYSIS OF HOST CELL PROTEINS

(71) Applicant: LONZA LTD, Visp (CH)

(72) Inventor: James Graham, Slough (GB)

(73) Assignee: LONZA LTD, Visp (CH)

(21) Appl. No.: 16/324,960

(22) PCT Filed: Aug. 11, 2017
(86) PCT No.: PCT/US17/46440

§ 371 Date: Feb. 12, 2019

Related U.S. Application Data
(60) Provisional application No. 62/374,489, filed on Aug. 12, 2016.

(51) Int. Cl. C12Q 1/37

(52) U.S. Cl. CPC C12Q 1/37; G16B 45/00

(57) ABSTRACT

Disclosed herein are methods and compositions useful for detecting and/or quantifying host cell proteins during the production of a product, e.g., a recombinant protein, e.g., an antibody.

### RELATED APPLICATIONS

This application claims priority to U.S. Ser. No. 62/374,489, filed Aug. 12, 2016, the entire contents of which is incorporated herein by reference.

### FIELD OF THE INVENTION

The present disclosure relates to methods of detecting and/or quantifying host cell protein impurities during the production of a product, e.g., a recombinant protein, e.g., an antibody.

### BACKGROUND

Host cell protein (HCP) is an unwanted complex mixture of host proteins which may present in the final product after various manufacturing process. Those HCPs can pose risks to, inter alia, product efficacy and patient safety.

Previously, HCP impurity testing has been accomplished using ELISA-based methods and/or advances in the field of proteomics. HCP impurity testing utilizing proteomics has been based on 2-dimensional chromatography separations. This approach is powerful, but lacks sufficient throughput to be used as a routine process development tool. Lack of such a tool means that proteomic HCP assessment has often been reactive and limited to analysis of a product following process development rather than as a source of information on which development decisions can be based. Therefore, a need exists for methods of detecting and quantifying HCPs in biopharmaceutical products in a simple, rapid, high throughput manner.

### SUMMARY

The invention pertains, in part, to the development of methods of rapidly analyzing a sample, e.g., a plurality of samples, comprising a protein, e.g., proteins, e.g., HCPs or fragments thereof, produced by processes or methods of manufacturing a product, e.g., recombinant polypeptide, to assess the risk the protein poses as a contaminant in a final formulated product, e.g., recombinant polypeptide. For example, a sample of recombinant polypeptide produced by a process or method of manufacturing may be analyzed by a method described herein to quickly assess the risk any protein, e.g., proteins, e.g., HCPs or fragments thereof, might pose as a contaminant. In such an exemplary method, many samples can be quickly and accurately assessed to assess the risk of many proteins, in such a way to allow for the comparison of the risk of contaminants associated with a process or method of manufacturing. A method of the invention may thus be useful to evaluate, differentiate, and select between processes or methods of manufacturing. Method of the invention may further be useful to evaluate, differentiate, and select between samples based on the assessment of the risk associated with proteins, as well as for monitoring a process or method of manufacturing to determine the ongoing development of proteins and associated contaminant risks.

In one aspect, the invention provides a simple method of rapidly analyzing a sample, e.g., to provide an assessment of the risk of a protein (e.g., the risk the protein presents if present as a contaminant in a preparation, e.g., a preparation to be administered to a subject, e.g., a pharmaceutical preparation), the method comprising:

a) providing, e.g., forming and/or maintaining, a sample mixture comprising:
i) the sample, which comprises the protein and optionally a product (e.g., a recombinant polypeptide, e.g., an antibody, enzyme, or cytokine), produced via a process; and
ii) a denaturant, e.g., deoxycholate and urea,

under conditions, e.g., concentrations of denaturant, that denatures the protein in the sample at temperature of between 10 and 30° C., e.g., 18-26° C., e.g., 20±3° C., 20±2° C., 20±1° C., or 20° C.;

b) providing, e.g., forming and or maintaining, a sample/enzyme mixture comprising:
i) sample mixture (e.g., an aliquot of sample mixture from (a)); and
ii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture, under conditions in which the enzyme maintains substantial activity and reacts with, e.g., cleaves, the protein to provide protein digestion products;

c) separating the protein digestion products using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products; and

d) assigning a protein risk score to a protein identified in the sample,

thereby analyzing the sample and providing an assessment of the risk of a protein.

In another aspect, the invention provides a simple method of rapidly analyzing a sample to provide an assessment of the risk of a protein, the method comprising:

a) providing, e.g., forming and/or maintaining, a sample mixture comprising:
i) the sample, which comprises the protein (e.g., a HCP) and optionally a therapeutic product (e.g., a recombinant polypeptide), produced via a process; and
ii) a first denaturant, e.g., guanidine hydrochloride,

under conditions, e.g., concentrations of a first denaturant, that denatures the protein in the sample at temperature of between 30 and 60° C., e.g., 45-55° C., e.g., 50±3° C., 50±2° C., 50±1° C., or 50° C.;

b) providing, e.g., forming and or maintaining, a sample/enzyme mixture comprising:
i) sample mixture;
ii) a second denaturant, e.g., urea; and
iii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;

c) separating the protein digestion products using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products;

d) assigning a protein risk score to a protein identified in the sample,

thereby analyzing the sample and providing an assessment of the risk of a protein.

In another aspect, the invention provides a method of evaluating a process of making a product, e.g., an evaluation that incorporates assessment of the risk presented by a protein other than the product, e.g., a contaminant, produced by the process, comprising:

a) providing, e.g., forming and/or maintaining, a sample mixture comprising:
i) the protein and optionally a product (e.g., a recombinant polypeptide, e.g., an antibody, enzyme, or cytokine), produced by the process; and
ii) a denaturant, e.g., deoxycholate and urea, or guanidine hydrochloride,

under conditions, e.g., concentrations of denaturant, that denatures the protein in the sample, e.g., at temperature of between 10 and 30° C., e.g., 18-26° C., e.g., 20±3° C., 20±2° C., 20±1° C., or 20° C.;

b) providing, e.g., forming and or maintaining, a sample/enzyme mixture comprising:
i) sample mixture (e.g., an aliquot of sample mixture from a)); and
ii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;

c) separating the protein digestion products, e.g., by using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products; and

d) assigning a protein risk score to a protein identified in the sample,

thereby evaluating a process of making a product, e.g., an evaluation that incorporates assessment of the risk presented by a protein other than the product, e.g., a contaminant, produced by the process.

In another aspect, the invention provides a method of evaluating a process of making a product, e.g., an evaluation that incorporates assessment of the risk presented by a protein other than the product, e.g., a contaminant, produced by the process, comprising:

a) providing, e.g., forming and/or maintaining, a sample mixture comprising:
i) the protein and optionally a product (e.g., a recombinant polypeptide, e.g., an antibody, enzyme, or cytokine), produced by the process; and
ii) a first denaturant, e.g., guanidine hydrochloride,

under conditions, e.g., concentrations of denaturant, that denatures the protein in the sample, e.g., at temperature of between 30 and 60° C., e.g., 45-55° C., e.g., 50±3° C., 50±2° C., 50±1° C., or 50° C.;

b) providing, e.g., forming and or maintaining, a sample/enzyme mixture comprising:
i) sample mixture (e.g., an aliquot of sample mixture from a));
ii) a second denaturant, e.g., urea; and
iii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;

c) separating the protein digestion products, e.g., by using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products; and

d) assigning a protein risk score to a protein identified in the sample,

thereby evaluating a process of making a product, e.g., an evaluation that incorporates assessment of the risk presented by a protein other than the product, e.g., a contaminant, produced by the process.

In another aspect, the invention provides a method of evaluating a method of manufacturing a product, e.g., a recombinant polypeptide, e.g., an antibody, enzyme, or cytokine, to provide an assessment of risk, (e.g., the risk presented by inclusion of a protein other than the product in a preparation of the product) comprising:

a) providing, e.g., forming and/or maintaining, a sample mixture comprising:
i) one or more proteins and the product, e.g., recombinant polypeptide, produced via the method of manufacturing; and
ii) a denaturant, e.g., deoxycholate and urea,

under conditions, e.g., concentrations of denaturant, that denatures the protein in the sample at temperature of between 10 and 30° C., e.g., 18-26° C., e.g., 20±3° C., 20±2° C., 20±1° C., or 20° C.;

b) providing, e.g., forming and or maintaining, a sample/enzyme mixture comprising:
i) sample mixture (e.g., an aliquot of sample mixture from (a)); and
ii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;

c) separating the protein digestion products using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products;

d) assigning a protein risk score to a protein identified in the sample;

optionally wherein (d) is repeated for a plurality of proteins, e.g., all proteins identified by the protein digestion products; and

e) assigning a process risk score to the method of manufacturing,

thereby evaluating the method of manufacturing a product, e.g., a recombinant polypeptide, to provide an assessment of risk.

In another aspect, the invention provides a method of evaluating a method of manufacturing a product, e.g., a recombinant polypeptide, e.g., an antibody, enzyme, or cytokine, to provide an assessment of risk, (e.g., the risk presented by inclusion of a protein other than the product in a preparation of the product) comprising:

a) providing, e.g., forming and/or maintaining, a sample mixture comprising:
i) one or more proteins and the product, e.g., recombinant polypeptide, produced via the method of manufacturing; and
ii) a first denaturant, e.g., guanidine hydrochloride,

under conditions, e.g., concentrations of denaturant, that denatures the protein in the sample at temperature of between 30 and 60° C., e.g., 45-55° C., e.g., 50±3° C., 50±2° C., 50±1° C., or 50° C.;

b) providing, e.g., forming and or maintaining, a sample/enzyme mixture comprising:
i) sample mixture (e.g., an aliquot of sample mixture from (a));
ii) a second denaturant, e.g., urea, and
iii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;

c) separating the protein digestion products using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products;

d) assigning a protein risk score to a protein identified in the sample;

optionally wherein (d) is repeated for a plurality of proteins, e.g., all proteins identified by the protein digestion products; and

e) assigning a process risk score to the method of manufacturing,

thereby evaluating the method of manufacturing a product, e.g., a recombinant polypeptide, to provide an assessment of risk.

In another aspect, the invention provides a method of manufacturing a product, e.g., a recombinant polypeptide, comprising providing a sample comprising the product, wherein the sample is analyzed by a method of analyzing a sample described herein.

In another aspect, the invention provides a database (e.g., memorialized or recorded on a computer readable medium) comprising a library of identifying characteristics for HCPs or protein digestion products and protein risk scores derived from cell culture supernatant of a cell culture (e.g., a CHO, eg., a GS-CHO, cell culture).

Among the advantages of the invention is that the methods disclosed herein allow for a simple, rapid risk assessment, e.g., immunogenicity assessment, dissociation, product stability, to be performed for any production system where the genome is known, or for specific variants of the production system (e.g., GS CHO specifically as a subset of CHO). Risk, e.g., immunogenicity, assessment can be performed for different patient populations (e.g. by geographic area or ethnicity). This is important since an overall average score for a protein contaminant, e.g., HCP, for the global population may mask a high score for a single particularly susceptible group. The risk, e.g., immunogenicity, calculation is fully integrated into the development process.

### BRIEF DESCRIPTION OF THE DRAWINGS

FIG. 1 is a top level overview of an analytical process according to the invention.

FIG. 2 is a graphical representation showing pI values for each protein quantified to determine if pDADMAC-mediated HCP removal was correlated with protein pI.

FIG. 3 is an LC-MS profile for the Protein SET.

FIG. 4 is a graph showing a comparison of overall product degradation risk score for different products and cell lines processed under different purification conditions.

FIG. 5 is a graph showing a comparison of phospholipase B abundance for different products and cell lines processed under different purification conditions.

FIG. 6 is a graph showing a comparison of cathep sin D abundance for different products and cell lines processed under different purification conditions.

FIG. 7 is a graph showing a comparison of overall immunogenicity risk score for different products and cell lines processed under different purification conditions.

FIG. 8 is a graph showing a comparison of total HCP abundance in culture supernatant treated with alternative protein removal methods.

FIG. 9 is a graph showing a comparison of product dissociation risk scores in culture supernatant treated with alternative protein removal methods.

FIG. 10 is a graph showing a comparison of product degradation risk scores in culture supernatant treated with alternative protein removal methods.

FIG. 11 is a graph showing a comparison of example specific HCP abundances in culture supernatant treated with alternative protein removal methods in comparison to total HCP levels.

FIG. 12 is a schematic showing methanol detoxification pathways and changes in protein expression in pAOX induction.

### DETAILED DESCRIPTION

For recombinant biopharmaceutical proteins to be acceptable for administration to human patients, it is important that residual contaminants resulting from the manufacture and purification process are removed from the final biological product, e.g., recombinant polypeptide. These process contaminants include culture medium proteins, immunoglobulin affinity ligands, viruses, endotoxin, DNA, and proteins, e.g., host cell proteins (HCPs). Contaminant proteins, e.g., HCPs, may generate a range of undesirable effects that may impact on the safety profile of a product, including immune response, adjuvant activity, direct biological activity or product interaction/degradation. These host cell contaminants include process-specific proteins, e.g., HCPs, which are process-related impurities/contaminants in the biologics derived from recombinant DNA technology (e.g., recombinant polypeptides).

U.S. and foreign regulations often require removal of such contaminants. For example, the U.S. Food and Drug Administration (FDA) requires that biopharmaceuticals intended for in vivo human use should be as free as possible of extraneous immunoglobulin and non-immunoglobulin impurities, and requires tests for detection and quantitation of potential impurities, such as proteinss, e.g., HCPs. As well, the International Conference on Harmonization (ICH) provides guidelines on test procedures and acceptance criteria for biotechnological/biological products.

Based mainly on the analytical challenge in detection and quantitation of impurity proteins present at trace levels, measurement of contaminant proteins, e.g., HCPs, in biotherapeutics has historically been performed on an aggregate basis using ELISA methodology, with results reported as a total grammage of proteins, e.g., HCPs, without information on identities and relative levels within the population. Contaminant protein, e.g., HCP, ELISAs are developed based on combinations of total protein and protein fractions from null-transfected cell lines and are performed on the inherent assumption that the contaminant proteins, e.g., HCP, load in the analyte varies only in abundance and not in composition.

Current proteomics based methods are limited in application as they do not have sufficient throughput to be used as a routine tool for manufacturing and process development, product monitoring, and analysis. Regulatory agencies require risk assessment to be associated with each contaminating protein, which existing proteomics based methods have not addressed in a rapid, scalable manner. Lack of such a rapid, high throughput tool means that proteomic protein, e.g., HCP, contaminant assessment is often reactive and limited to analysis of a small number of samples in process development rather than as a routine tool on which development decisions can be based.

A particular problem that manufacturers of biopharmaceuticals face is that each protein, e.g., HCP, has a specific, individual activity resulting in highly variable risk profiles for different proteins, e.g., HCP, populations at similar total abundances. The mixture of proteins, e.g., HCPs, that are present as impurities can vary substantially between products, especially as it is know that proteins, e.g., HCPs, can “piggyback” through purification by direct binding to the protein therapeutic. This can result in a number of issues including product and surfactant degradation, adjuvant activity and adverse immune response when the therapeutic is administered.

The invention allows the identification of which specific proteins, e.g., HCPs, are present in a protein therapeutic at all manufacturing scales, as well as all manufacturing and purification stages of the therapeutic protein and with sufficient throughput to support routine process development.

The present disclosure describes a proteomic analysis of proteins, e.g., HCPs, in products, e.g., purified therapeutic products or recombinant polypeptides, using proteomics in a format compatible with commercial demands, including e.g., manufacturing process development, production monitoring, and analysis of final products.

This process incorporates an integrated clinical risk assessment for each protein, e.g., HCP, based on in silico prediction of immunogenicity. These predictions are based on the specific production system and can be made against defined patient sub-populations that may be particularly susceptible to certain protein, e.g., HCP, epitopes (e.g. by geographic area or ethnicity). The specific combination of high-throughput analysis methods and integrated impurity risk assessment allows routine decisions to be made based on calculated clinical risk during the manufacturing process development. These conclusions can be made with high confidence as both the specific genome of the production system and the full range of potential patient responses are taken into account.

The proteomic based analysis of proteins, e.g., HCPs, disclosed herein can be used throughout the entire manufacturing process, thus avoiding development of different assays to detect proteins, e.g., HCPs, during scalablity of manufacturing, i.e., the method is independent of scale. The use of the same monitoring method throughout the entire manufacturing process from small scale to large scale avoids multiple issues such as the need to develop multiple monitoring methods at different manufacturing scales, along with concomitant errors in translation of the output from one scale to another scale. Rather, the proteomic method provides a simple, consistent, reproducible, rapid method that can be used independent of manufacturing scale. It also ensures that the same set of proteins, e.g., HCPs, are monitored at different stages of the process to show the changes in proteins, e.g., HCPs, for example, during cell culture, protein expression, as well as before and after purification.

The present disclosure describes, inter alia, methods of analyzing hundreds of samples (e.g., samples derived from processes and methods of manufacturing products, e.g., recombinant polypeptides or therapeutic products) quickly by optimizing key steps of proteomic analysis, such as, for example:

applying particular combinations and levels of denaturing agents, e.g., deoxycholate, urea, and guanidine hydrochloride;

using a number of different enzymes, e.g., proteolytic enzymes, at select pH (e.g., low pH, e.g., acidic conditions), as opposed to only individual enzymes, e.g., proteolytic enzyme; and

omission of alkylation step(s) without loss of result quality.

The methods of the present disclosure are capable of detecting, identifying, and quantifying the abundance of thousands of proteins (e.g., HCPs) present at very low levels in sample mixtures. The methods of the present disclosure are applicable at the start of manufacturing and processes, e.g., using cell supernatant, rather than only being suitable for analyzing purified product, e.g., recombinant polypeptide.

The methods of the present disclosure can be performed significantly faster and on a larger number of samples than commonly used methods. Exemplary methods of the present disclosure can analyze 100 samples in 3 to 5 days, whereas commonly used methods require 5-10 days to analyze 6 samples. Conservatively this represents at least a 10-fold throughput increase. In one embodiment, throughput is improved at least by about 10-fold, 20-fold, 30-fold, 40-fold, 50-fold, 60-fold, 70-fold, 80-fold, or 90-fold.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “a cell” can mean one cell or more than one cell.

As used herein, the term “protein” or “proteins” in the context of a sample refers to any protein in a sample produced by a process or method of manufacturing that is not the desired product, e.g., the desired recombinant product, e.g., therapeutic product. In some embodiments, proteins may be host cell proteins (HCPs) or fragments thereof.

As used herein, the term “host cell protein” or “HCP” refers to any protein produced or encoded by the organism used to produce a recombinant polypeptide product and unrelated to the intended recombinant product. HCPs are undesirable in the final drug substance.

As used herein, the term “semi-quantitative” refers to the comparative assessment of different chemical species by mass spectrometry without reference to specific standards for each individual species.

As used herein, the term “endogenous” refers to any material from or naturally produced inside an organism, cell, tissue or system.

As used herein, the term “exogenous” refers to any material introduced to or produced outside of an organism, cell, tissue or system. Accordingly, “exogenous nucleic acid” refers to a nucleic acid that is introduced to or produced outside of an organism, cell, tissue or system. In an embodiment, sequences of the exogenous nucleic acid are not naturally produced, or cannot be naturally found, inside the organism, cell, tissue, or system that the exogenous nucleic acid is introduced into. In one embodiment, the sequences of the exogenous nucleic acids are non-naturally occurring sequences, or encode non-naturally occurring products.

As used herein, the term “heterologous” refers to any material from one species, when introduced to an organism, cell, tissue or system from a different species.

As used herein, the terms “nucleic acid,” “polynucleotide,” or “nucleic acid molecule” are used interchangeably and refers to deoxyribonucleic acid (DNA) or ribonucleic acid (RNA), or a combination of a DNA or RNA thereof, and polymers thereof in either single- or double-stranded form. The term “nucleic acid” includes, but is not limited to, a gene, cDNA, or an mRNA. In one embodiment, the nucleic acid molecule is synthetic (e.g., chemically synthesized or artificial) or recombinant. Unless specifically limited, the term encompasses molecules containing analogues or derivatives of natural nucleotides that have similar binding properties as the reference nucleic acid and are metabolized in a manner similar to naturally or non-naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the terms “peptide,” “polypeptide,” and “protein” (e.g., protein when not used in the context of a method of the present invention) are used interchangeably, and refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. In one embodiment, a protein may comprise of more than one, e.g., two, three, four, five, or more, polypeptides, in which each polypeptide is associated to another by either covalent or non-covalent bonds/interactions. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others.

As used herein, “product” refers to a molecule, nucleic acid, polypeptide, or any hybrid thereof, that is produced, e.g., expressed, by a cell which has been modified or engineered to produce the product. In one embodiment, the product is a naturally occurring product or a non-naturally occurring product, e.g., a synthetic product. In one embodiment, a portion of the product is naturally occurring, while another portion of the product is non-naturally occurring. In one embodiment, the product is a polypeptide, e.g., a recombinant polypeptide. In one embodiment, the product is suitable for diagnostic or pre-clinical use. In another embodiment, the product is suitable for therapeutic use, e.g., for treatment of a disease. In one embodiment, the product is selected from Table 1 or Table 2. In one embodiment, the modified or engineered cells comprise an exogenous nucleic acid that controls expression or encodes the product. In other embodiments, the modified or engineered cells comprise other molecules, e.g., that are not nucleic acids, that controls the expression or construction of the product in the cell.

In one embodiment, the modification of the cell comprises the introduction of an exogenous nucleic acid comprising a nucleic acid sequence that controls or alters, e.g., increases, the expression of an endogenous nucleic acid sequence, e.g., endogenous gene. In such embodiments, the modified cell produces an endogenous polypeptide product that is naturally or endogenously expressed by the cell, but the modification increases the production of the product and/or the quality of the product as compared to an unmodified cell, e.g., as compared to endogenous production or quality of the polypeptide.

In another embodiment, the modification of the cell comprises the introduction of an exogenous nucleic acid encoding a recombinant polypeptide as described herein. In such embodiments, the modified cell produces a recombinant polypeptide product that can be naturally occurring or non-naturally occurring. In such embodiments, the modified cell produces a recombinant polypeptide product that can also be endogenously expressed by the cell or not. In embodiments where the recombinant polypeptide product is also endogenously expressed by the cell, the modification increases the production of the product and/or the quality of the product as compared to an unmodified cell, e.g., as compared to endogenous production or quality of the polypeptide.

As used herein, “recombinant polypeptide” or “recombinant protein” refers to a polypeptide that can be produced by a cell described herein. A recombinant polypeptide is one for which at least one nucleotide of the sequence encoding the polypeptide, or at least one nucleotide of a sequence which controls the expression of the polypeptide, was formed by genetic engineering (of the cell or of a precursor cell). E.g., at least one nucleotide was altered, e.g., it was introduced into the cell or it is the product of a genetically engineered rearrangement. In an embodiment, the sequence of a recombinant polypeptide does not differ from a naturally occurring isoform of the polypeptide or protein. In an embodiment, the amino acid sequence of the recombinant polypeptide differs from the sequence of a naturally occurring isoform of the polypeptide or protein. In an embodiment, the recombinant polypeptide and the cell are from the same species. In an embodiment, the recombinant polypeptide is endogenous to the cell, in other words, the cell is from a first species and the recombinant polypeptide is native to that first species. In an embodiment, the amino acid sequence of the recombinant polypeptide is the same as or is substantially the same as, or differs by no more than 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% from, a polypeptide encoded by the endogenous genome of the cell. In an embodiment, the recombinant polypeptide and the cell are from different species, e.g., the recombinant polypeptide is a human polypeptide and the cell is a non-human, e.g., a rodent, e.g., a CHO, or an insect cell. In an embodiment, the recombinant polypeptide is exogenous to the cell, in other words, the cell is from a first species and the recombinant polypeptide is from a second species.

In one embodiment, the polypeptide is a synthetic polypeptide. In one embodiment, the polypeptide is derived from a non-naturally occurring source. In an embodiment, the recombinant polypeptide is a human polypeptide or protein which does not differ in amino acid sequence from a naturally occurring isoform of the human polypeptide or protein. In an embodiment, the recombinant polypeptide differs from a naturally occurring isoform of the human polypeptide or protein at no more than 1, 2, 3, 4, 5, 10, 15 or 20 amino acid residues. In an embodiment, the recombinant polypeptide differs from a naturally occurring isoform of the human polypeptide by no more than 1, 2, 3, 4, 5, 6, 7, 8, 9, 10, or 15% of its amino acid residues.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Acquiring a sample” as the term is used herein, refers to obtaining possession of a sample, e.g., a tissue sample or nucleic acid sample, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third party laboratory that directly acquired the sample). Directly acquiring a sample includes performing a process that includes a physical change in a physical substance, e.g., a starting material, such as a tissue, e.g., a tissue in a human patient or a tissue that has was previously isolated from a patient. Exemplary changes include making a physical entity from a starting material, dissecting or scraping a tissue; separating or purifying a substance (e.g., a sample tissue or a nucleic acid sample); combining two or more separate entities into a mixture; performing a chemical reaction that includes breaking or forming a covalent or non-covalent bond. Directly acquiring a sample includes performing a process that includes a physical change in a sample or another substance, e.g., as described above.

As used herein, “protein digestion products” refer to fragments of proteins, e.g., peptides, produced by the action of an enzyme, e.g., a proteolytic enzyme, for which the protein is a substrate. Examples of enzymes include trypsin, lysC, GluC, AspN, and others known in the art.

As used herein, a “protein risk score” comprises an assessment of the risk associated with a protein, e.g., a HCP or fragment thereof, as a contaminant or impurity in a product, e.g, a therapeutic product, e.g., a final formulation of a therapeutic product. The protein risk score may be a function of one or more of: an unwanted, e.g., off-target, property in a subject receiving a preparation comprising the protein and the product, e.g., immunogenicity, e.g., an unwanted immune response; an unwanted effect of the protein in a preparation of the product, e.g., a preparation of a drug, e.g., the propensity to cause denaturation, precipitation, color, or odor; and a value for the abundance of the protein present in the sample. For example, a sample, optionally comprising product, produced by a process or method of manufacturing, comprises one or more protein contaminants. A method of the invention may analyze the proteins or fragments thereof within the sample, and assign one or more protein risk scores to each protein identified in the sample. In some embodiments, a protein risk score is or comprises a value produced by Epibase®, e.g., a immunogenicity score. In some embodiments, a protein risk score is a value of the protein abundance in the sample.

As used herein a “process risk score” comprises an assessment of the risk associated with a process or method of manufacturing, and is a function of the protein risk scores of proteins present in samples produced by the process or method of manufacturing. For example, sample A could be produced by process A and sample B could be produced by process B. Using the methods of the invention, samples A and B could be analyzed, the protein risk scores of their protein contaminants assessed, and the process risk scores of processes A and B determined, allowing for the rapid comparison of processes A and B.

As used herein, a “process” is a series of one or more operations and/or conditions that produces a sample comprising, inter alia, a protein or plurality of proteins, e.g., a HCP or fragment thereof. In some embodiments, the sample further comprises a product, e.g., a recombinant polypeptide, or a therapeutic product. For example, an exemplary process could comprise culturing a plurality of cells under conditions conducive to the expression of a recombinant polypeptide, thus producing a sample, e.g., cell culture, supernatant, or cell lysate, comprising one or more proteins, e.g., HCPs or fragments thereof, and product, e.g., recombinant polypeptide.

As used herein, a “method of manufacturing” is a series of one or more operations and/or conditions that produces a sample comprising a product, e.g., a recombinant polypeptide or a therapeutic product, and a protein or plurality of proteins, e.g., a HCP or fragment thereof. For example, an exemplary method of manufacturing could comprise culturing a plurality of cells under conditions conducive to the expression of a recombinant polypeptide, thus producing a sample, e.g., cell culture, supernatant, or cell lysate, comprising one or more proteins, e.g., HCPs or fragments thereof, and product, e.g., recombinant polypeptide.

As used herein, MS¹ means mass spectrometry.

As used herein, MS² means tandem mass spectrometry.

As used herein, “substantially active” or “substantial activity” describes enzyme, e.g., a plurality of enzymes, e.g., in a sample under a set of conditions or in a step of a method or process described herein, that are at least 50, 60, 70, 80, 90, or 100% active, e.g., operating at 50, 60, 70, 80, 90, or 100% efficiency/reaction rate compared to a reference efficiency/reaction rate, e.g., the highest efficiency/reaction rate of that enzyme under ideal conditions, e.g., conditions recommended by the enzyme supplier or conditions without denaturant, reducing agent, or non-recommended pH present.

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

### Sample Preparation

The methods described herein, in part, recite methods for analyzing samples, e.g., samples comprising proteins, e.g. HCPs, generated by a process or method of manufacturing, e.g., samples comprising cell culture, supernatant, or cell lysate. In some embodiments, the methods described herein recite preparing the sample for analysis. In some embodiments, the analysis comprises mass spectrometry, identification of the proteins, e.g. HCPs within the sample, and/or assignment of risk scores to the proteins, e.g. HCPs and/or the process/method of manufacturing that produced the sample.

In some embodiments, preparing the sample for analysis may comprise exposing the proteins, e.g. HCPs to a denaturant. In some embodiments, the denaturant is a chaotropic agent, an acid, a base, a reducing agent, or a detergent. In some embodiments, the denaturant is selected from guanidine hydrochloride, urea, and deoxycholate. In some embodiments, the denaturant is a combination of multiple types of denaturants or multiple denaturants, e.g., urea and deoxycholate, or guanidine hydrochloride and urea. In some embodiments, preparing the sample for analysis comprises multiple steps wherein the denaturant provided in one step is different from the denaturant provided in a second step, or the concentration of a denaturant changes, e.g., is altered, from step to step. In some embodiments, the multiple steps of sample preparation may alter, e.g., decrease, e.g., by dilution, the concentration of a first denaturant, e.g., guanidine hydrochloride, and introduce or increase the concentration of a second denaturant, e.g., urea. In some embodiments, the concentration of denaturant in the sample mixture is at least 1, 1.5, 2, 2.5, 3, 3.5, 4, 4.5, 5, 5.5, 6, 6.5, 6.6, 6.7, 6.8, 6.9, 7, 7.5, or 8 M. In some embodiments, the concentration of denaturant, e.g., guanidine hydrochloride, in the sample mixture is 1-10 M, 2-9 M, 3-8 M, 4-7 M, 5-7 M, 6-6.6 M, 6 M, 6.6 M, or 8 M. In some embodiments, the concentration of denaturant, e.g., urea, in the sample mixture is 0-10 M, 2-9 M, 3-8 M, 4-7 M, 5-7 M, 0.5-5 M, 0.5-2 M, 0.5 M, 1 M, or 2 M. In some embodiments, the concentration of denaturant, e.g., deoxycholate, in the sample mixture is at least 0.01%, 0.05%, 0.1%, 0.2%, 0.5%, 0.7%, 0.9%, 1%, 1.2%, 1.5%, 2%, 5%, 10%, 15%, 20%, 30%, 40%, or 50% (m/v), e.g, 0.1%-2%. In some embodiments, the concentration of denaturant, e.g., deoxycholate, in the sample mixture is 0.01%-50%, 1%-40%, 1%-20%, 0.5%-10%, 0.01%-5%, or 0.1%-2% (m/v). In some embodiments, the sample (e.g., sample mixture) comprises both urea and deoxycholate. In some embodiments, the sample (e.g., sample mixture) comprises both urea and deoxycholate and the concentration of urea is at least 8 M (e.g., 8 M) and the concentration of deoxycholate is at least 1% (e.g., 1%) (m/v). In some embodiments, the sample (e.g., sample mixture) comprises both urea and deoxycholate, and the concentration of urea is at least 8 M (e.g., 8 M) and the concentration of deoxycholate is at least 0.01% (e.g., 0.01%) (m/v). In some embodiments, the concentration of denaturant in the sample/enzyme mixture is less than or equal to 4, 3.5, 3, 2.5, 2, 1.5, 1, 0.5, or 0.1 M. In some embodiments, the concentration of denaturant, e.g., guanidine hydrochloride, in the sample/enzyme mixture is less than or equal to 0.5 or 0.1 M, e.g., essentially 0 M. In some embodiments, the concentration of denaturant, e.g., urea, in the sample/enzyme mixture is less than or equal to 4, 3.5, 3, 2.5, 2, 1.5, 1, 0.5, or 0.1 M, e.g., less than or equal to 2 M or less than or equal to 0.5 M. In some embodiments, the concentration of denaturant, e.g., deoxycholate, in the sample/enzyme mixture is less than or equal to 2%, 1%, 0.1%, 0.05%, 0.01%, 0.005%, 0.0025%, 0.001%, or 0.0001%, e.g., essentially 0% (m/v). In some embodiments, the sample/enzyme mixture comprises both urea and deoxycholate. In some embodiments, the sample/enzyme mixture comprises both urea and deoxycholate, and the concentration of urea is less than or equal to 2 M (e.g., 2 M or 0.5 M) and the concentration of deoxycholate is less than or equal to 0.0025% (m/v). In some embodiments, samples, e.g., sample mixtures and/or sample/enzyme mixtures, comprising deoxycholate also comprise acetonitrile, e.g., at least 10, 20, 30, 40, 50, 60 70, 80, or 90% acetonitrile (e.g., 80% acetonitrile) (m/v).

In some embodiments, preparing the sample for analysis may comprise exposing the protein to a reducing agent. Disulfide bonds present in the protein may need to be reduced to denature the protein. Reducing agents contemplated are those known in the art and include Tris(2-carboxyethyl)phosphine (TCEP), dithiothreitol (DTT), or beta-mercaptoethanol (i.e. 2-mercaptoethanol). In some embodiments, the concentration of reducing agent in the sample mixture is at least 1, 2, 3, 4, 5, 6, 7, 8, 9, 10, 11, 12, 13, 14, 15, 16, 17, 18, 19, or 20 mM, e.g., at least 10 mM, e.g., 10 mM. In some embodiments, the concentration of reducing agent in the sample/enzyme mixture is less than or equal to 10, 9, 8, 7, 6, 5, 4, 3, 2, 1, 0.5, or 0.1 mM, e.g., less than or equal to 1 mM, e.g., 1 mM.

In some embodiments, preparing the sample for analysis may not comprise exposing the protein to an alkylating agent. Alkylating agents, e.g., iodoacetamide, can be used to alkylate cysteine thiol groups as one way of preventing reformation of disulfide bonds. While not wishing to be bound by theory, maintaining the presence of a reducing agent, e.g., a low concentration of a reducing agent (e.g., 1 mM reducing agent, e.g., 1 mM TCEP), throughout sample preparation may mitigate the need for an alkylating agent, e.g., to contribute to denaturation of a protein. In addition, skipping an alkylating step may expedite sample preparation time.

In some embodiments, preparing the sample for analysis may comprise providing a specific pH in the sample mixture or sample/enzyme mixture. In some embodiments, the pH of the sample mixture is 5, 5.25, 5.5, 5.75, 6, 6.25, 6.5, 6.75, 7, 7.25, 7.5, or 8. In some embodiments, the pH of the sample mixture is 5.5. In some embodiments, the pH of the sample/enzyme mixture is 6, 6.25, 6.5, 6.75, 6.8, 6.9, 7, 7.1, 7.2, 7.3, 7.4, 7.5, 7.75, 8, 8.25, or 8.5. In some embodiments, the pH of the sample/enzyme mixture is 7, 7.2, 7.3, or 8. In some embodiments, the pH of the sample/enzyme mixture is 7.3. Applicant unexpectedly discovered that in using the methods of the invention a less than optimum pH for an enzyme reaction could be used with minimum effect on the reaction of the enzyme but an increased speed in the overall analysis, e.g., from 6 sample runs to 96 or 192 sample runs, as well as performing the entire analysis in 3-5 days (e.g., 3 days, 4 days, or 5 days).

In some embodiments, sample preparation is conducted at room temperature. In some embodiments, sample preparation, e.g., conditions for denaturation, is conducted at between 30 and 60° C., e.g., 45-55° C., e.g., 50±3° C., 50±2° C., 50±1° C., or 50° C. In some embodiments, sample preparation, e.g., conditions for denaturation, is conducted at between 10 and 30° C., e.g., 18-26° C., e.g., 20±3° C., 20±2° C., 20±1° C., or 20° C.

In some embodiments, preparing the sample for analysis may comprise providing an enzyme, e.g., a proteolytic enzyme, in the sample/enzyme mixture. The enzyme, e.g., proteolytic enzyme, may be trypsin, lysC, GluC, AspN, or other enzymes known in the art; methods of use and characteristics of said enzymes are also available in the art. In some embodiments, the enzyme, e.g., proteolytic enzyme, is present in a 1:100, 1:90, 1:80, 1:70, 1:60, 1:50, 1:40, 1:30, 1:20, 1:15, 1:10, 1:8, 1:6, 1:5, 1:4, 1:3, 1:2, or 1:1 ratio (enzyme to protein). In some embodiments, the enzyme, e.g., proteolytic enzyme, is present in a 1:20 ratio (enzyme to protein). In some embodiments, the enzyme, e.g., proteolytic enzyme, is present in a 1:40 ratio (enzyme to protein).

### One-Dimensional Chromatography

Methods of 1-dimensional (1D) chromatography suitable for use in the methods described here are known to one of skill in the art and include, e.g., affinity chromatography, gel filtration chromatography, ion exchange chromatography, reversed phase chromatography, hydrophobic interaction chromatography. In some embodiments, the one-dimensional chromatography method is HPLC reversed phase chromatography. Chromatography can include high performance liquid chromatography (HPLC), gas chromatography (GC), capillary electrophoresis, ion mobility. See also, e.g., Process Scale Purification of Antibodies, Uwe Gottschalk 2011 John Wiley & Sons ISBN: 1118210743; Antibodies Vol 1 Production and Purification, G. Subramanian 2013 Springer Science & Business Media; Basic Methods in Antibody Production and Characterization, Gary C. Howard 2000 CRC Press.

Additional exemplary chromatographic methods include, but are not limited to, Strong Anion Exchange chromatography (SAX), liquid chromatography (LC), high performance liquid chromatography (HPLC), ultra performance liquid chromatography (UPLC), thin layer chromatography (TLC), amide column chromatography, and combinations thereof. Exemplary mass spectrometry (MS) include, but are not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof. Exemplary electrophoretic methods include, but are not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof. Exemplary nuclear magnetic resonance (NMR) include, but are not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

### Mass Spectrometry

Mass spectrometry methods suitable for use in the methods described herein are known to one of skill in the art and include, e.g., electrospray ionization MS, matrix-assisted laser desportion/ionization MS, time of flight MS, fourier-transform ion cyclotron resonance MS, quadrupole time of flight MS, linear quadrupole, quadrupole ion trap MS, orbitrap, cylindrical ion trap, three dimensional ion trap, quadruple mass filter, tandem mass spectrometry. In some embodiments, the mass spectrometry is tandem mass spectrometry. See also, e.g., Protein Mass Spectrometry, Julian Whitelegge 2008, Elsevier; Protein Sequencing and Identification Using Tandem Mass Spectrometry, Michael Kinter 2005, John Wiley & Sons; Characterization of Protein Therapeutics using Mass Spectrometry, Guodong Chen 2014, Springer Science & Business Media.

### Production Parameters

A production parameter as used herein is a parameter or element in a production process. Production parameters that can be selected include, e.g., the cell or cell line used to produce the glycoprotein preparation, the culture medium, culture process or bioreactor variables (e.g., batch, fed-batch, or perfusion), purification process and formulation of a glycoprotein preparation. Primary production parameters include: 1) the types of host; 2) genetics of the host; 3) media type; 4) fermentation platform; 5) purification steps; and 6) formulation. Secondary production parameter, as used herein, is a production parameter that is adjustable or variable within each of the primary production parameters. Examples include: selection of host subclones based on desired glycan properties; regulation of host gene levels constitutive or inducible; introduction of novel genes or promoter elements; media additives (e.g. partial list on Table IV); physiochemical growth properties; growth vessel type (e.g. bioreactor type, T flask); cell density; cell cycle; enrichment of product with a desired glycan type (e.g. by lectin or antibody-mediated enrichment, ion-exchange chromatography, CE, or similar method); or similar secondary production parameters clear to someone skilled in the art.

Media

The methods described herein can include determining and/or selecting a media component and/or the concentration of a media component that has a positive correlation to a desired glycan property or properties. A media component can be added in or administered over the course of glycoprotein production or when there is a change media, depending on culture conditions. Media components include components added directly to culture as well as components that are a byproduct of cell culture.

Media components include, e.g., buffer, amino acid content, vitamin content, salt content, mineral content, serum content, carbon source content, lipid content, nucleic acid content, hormone content, trace element content, ammonia content, co-factor content, indicator content, small molecule content, hydrolysate content and enzyme modulator content.

Examples of various media components are provided below:

| | |
|---|---|
| amino acids | sugar precursors |
| Vitamins | Indicators |
| Carbon source (natural and unnatural) | Nucleosides or nucleotides |
| Salts | butyrate or organics |
| Sugars | DMSO |
| Sera | Animal derived products |
| Plant derived hydrolysates | Gene inducers |
| sodium pyruvate | Non natural sugars |
| Surfactants | Regulators of intracellular pH |
| Ammonia | Betaine or osmoprotectant |
| Lipids | Trace elements |
| Hormones or growth factors | minerals |
| Buffers | Non natural amino acids |
| Non natural amino acids | Non natural vitamins |

Exemplary buffers include Tris, Tricine, HEPES, MOPS, PIPES, TAPS, bicine, BES, TES, cacodylate, MES, acetate, MKP, ADA, ACES, glycinamide and acetamidoglycine. The media can be serum free or can include animal derived products such as, e.g., fetal bovine serum (FBS), fetal calf serum (FCS), horse serum (HS), human serum, animal derived serum substitutes (e.g., Ultroser G, SF and HY; non-fat dry milk; Bovine EX-CYTE), fetuin, bovine serum albumin (BSA), serum albumin, and transferrin. When serum free media is selected lipids such as, e.g., palmitic acid and/or steric acid, can be included.

Lipids components include oils, saturated fatty acids, unsaturated fatty acids, glycerides, steroids, phospholipids, sphingolipids and lipoproteins. Exemplary amino acid that can be included or eliminated from the media include alanine, arginine, asparagine, aspartic acid, cysteine, glutamic acid, glutamine, glycine, histidine, proline, isoleucine, leucine, lysine, methionine, phenylalanine, proline, serine, threonine, tryptophan, tyrosine and valine. Examples of vitamins that can be present in the media or eliminated from the media include vitamin A (retinoid), vitamin B1 (thiamine), vitamin B2 (riboflavin), vitamin B3 (niacin), vitamin B5 (pantothenic acid), vitamin B6 (pyroxidone), vitamin B7 (biotin), vitamin B9 (folic acid), vitamin. B12 (cyanocobalamin), vitamin C (ascorbic acid), vitamin D, vitamin E, and vitamin K.

Minerals that can be present in the media or eliminated from the media include bismuth, boron, calcium, chlorine, chromium, cobalt, copper, fluorine, iodine, iron, magnesium, manganese, molybdenum, nickel, phosphorus, potassium, rubidium, selenium, silicon, sodium, strontium, sulfur, tellurium, titanium, tungsten, vanadium, and zinc. Exemplary salts and minerals include CaCl2 (anhydrous), CuSO4 5H2O, Fe(NO3).9H2O, KCl, KNO3, KH2PO4, MgSO4 (anhydrous), NaCl, NaH2PO4H2O, NaHCO₃, Na2SE3 (anhydrous), ZnSO4.7H2O; linoleic acid, lipoic acid, D-glucose, hypoxanthine 2Na, phenol red, putrescine 2HCl, sodium pyruvate, thymidine, pyruvic acid, sodium succinate, succinic acid, succinic acid.Na.hexahydrate, glutathione (reduced), para-aminobenzoic acid (PABA), methyl linoleate, bacto peptone G, adenosine, cytidine, guanosine, 2′-deoxyadenosine HCl, 2′-deoxycytidine HCl, 2′-deoxyguanosine and uridine. When the desired glycan characteristic is decreased fucosylation, the production parameters can include culturing a cell, e.g., CHO cell, e.g., dhfr deficient CHO cell, in the presence of manganese, e.g., manganese present at a concentration of about 0.1 μM to 50 μM. Decreased fucosylation can also be obtained, e.g., by culturing a cell (e.g., a CHO cell, e.g., a dhfr deficient CHO cell) at an osmolality of about 350 to 500 mOsm. Osmolality can be adjusted by adding salt to the media or having salt be produced as a byproduct as evaporation occurs during production.

Hormones include, for example, somatostatin, growth hormone-releasing factor (GRF), insulin, prolactin, human growth hormone (hGH), somatotropin, estradiol, and progesterone. Growth factors include, for example, bone morphogenic protein (BMP), epidermal growth factor (EGF), basic fibroblast growth factor (bFGF), nerve growth factor (NGF), bone derived growth factor (BDGF), transforming growth factor-beta1 (TGF-beta1), [Growth factors from U.S. Pat. No. 6,838,284 B2], hemin and NAD. Examples of surfactants that can be present or eliminated from the media include Tween-80 and pluronic F-68. Small molecules can include, e.g., butyrate, ammonia, non natural sugars, non natural amino acids, chloroquine, and betaine.

Physiochemical Parameters

Production parameters can also include physiochemical parameters. Such conditions can include temperature, pH, osmolality, shear force or agitation rate, oxidation, spurge rate, growth vessel, tangential flow, DO, CO₂, nitrogen, fed batch, redox, cell density and feed strategy. Examples of physiochemical parameters that can be selected include, e.g., pH, osmolality, shear force or agitation rate, oxidation, spurge rate, growth vessel, tangential flow, batch dissolved O2, CO₂, nitrogen, fed batch, redox, cell density, perfusion culture, feed strategy, temperature and time of culture.

Additional production parameters are known to one of skill in the art, see e.g., Antibody Expression and Production (2011) Ed. Mohamed Al-Rubeai; Springer Publishing.

Products and Nucleic Acids Encoding them

Provided herein are methods for identifying, selecting, or making a cell or cell line capable of producing a product, e.g., cells and products as recited in the methods of the invention. The products encompassed by the present disclosure include, but are not limited to, molecules, nucleic acids, polypeptides (e.g., recombinant polypeptides, e.g., antibodies, bispecific antibodies, multispecific antibodies), or hybrids thereof, that can be produced by, e.g., expressed in, a cell. In some embodiments, the cells are engineered or modified to produce the product. Such modifications include the introducing molecules that control or result in production of the product. For example, a cell is modified by introducing an exogenous nucleic acid that encodes a polypeptide, e.g., a recombinant polypeptide, and the cell is cultured under conditions suitable for production, e.g., expression and secretion, of the polypeptide, e.g., recombinant polypeptide.

In embodiments, the cultured cells are used to produce proteins e.g., antibodies, e.g., monoclonal antibodies, and/or recombinant proteins, for therapeutic use. In embodiments, the cultured cells produce peptides, amino acids, fatty acids or other useful biochemical intermediates or metabolites. For example, in embodiments, molecules having a molecular weight of about 4000 daltons to greater than about 140,000 daltons can be produced. In embodiments, these molecules can have a range of complexity and can include posttranslational modifications including glycosylation.

In embodiments, the polypeptide is, e.g., BOTOX, Myobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-n1, DL-8234, interferon, Suntory (gamma-1a), interferon gamma, thymosin alpha 1, tasonermin, DigiFab, ViperaTAb, EchiTAb, CroFab, nesiritide, abatacept, alefacept, Rebif, eptoterminalfa, teriparatide, calcitonin, etanercept, hemoglobin glutamer 250 (bovine), drotrecogin alpha, collagenase, carperitide, recombinant human epidermal growth factor, DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin, Indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (Hydron), deslorelin, histrelin, nafarelin, leuprolide (ATRIGEL), leuprolide (DUROS), goserelin, Eutropin, somatropin, mecasermin, enlfavirtide, Org-33408, insulin glargine, insulin glulisine, insulin (inhaled), insulin lispro, insulin deternir, insulin (RapidMist), mecasermin rinfabate, anakinra, celmoleukin, 99 mTc-apcitide, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex′ recombinant human luteinizing hormone, dornase alpha, trafermin, ziconotide, taltirelin, diboterminalfa, atosiban, becaplermin, eptifibatide, Zemaira, CTC-111, Shanvac-B, octreotide, lanreotide, ancestirn, agalsidase beta, agalsidase alpha, laronidase, prezatide copper acetate, rasburicase, ranibizumab, Actimmune, PEG-Intron, Tricomin, recombinant human parathyroid hormone (PTH) 1-84, epoetin delta, transgenic antithrombin III, Granditropin, Vitrase, recombinant insulin, interferon-alpha, GEM-21S, vapreotide, idursulfase, omnapatrilat, recombinant serum albumin, certolizumab pegol, glucarpidase, human recombinant Cl esterase inhibitor, lanoteplase, recombinant human growth hormone, enfuvirtide, VGV-1, interferon (alpha), lucinactant, aviptadil, icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide, tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone, recombinant G-CSF, insulin, insulin (Technosphere), insulin (AERx), RGN-303, DiaPep277, interferon beta, interferon alpha-n3, belatacept, transdermal insulin patches, AMG-531, MBP-8298, Xerecept, opebacan, AIDSVAX, GV-1001, LymphoScan, ranpirnase, Lipoxysan, lusupultide, MP52, sipuleucel-T, CTP-37, Insegia, vitespen, human thrombin, thrombin, TransMlD, alfimeprase, Puricase, terlipressin, EUR-1008M, recombinant FGF-I, BDM-E, rotigaptide, ETC-216, P-113, MBI-594AN, duramycin, SCV-07, OPI-45, Endostatin, Angiostatin, ABT-510, Bowman Birk Inhibitor, XMP-629, 99 mTc-Hynic-Annexin V, kahalalide F, CTCE-9908, teverelix, ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, SB-249553, Oncovax-CL, OncoVax-P, BLP-25, CerVax-16, MART-1, gp100, tyrosinase, nemifitide, rAAT, CGRP, pegsunercept, thymosinbeta4, plitidepsin, GTP-200, ramoplanin, GRASPA, OBI-1, AC-100, salmon calcitonin (eligen), examorelin, capromorelin, Cardeva, velafermin, 131I-TM-601, KK-220, T-10, ularitide, depelestat, hematide, Chrysalin, rNAPc2, recombinant Factor V111 (PEGylated liposomal), bFGF, PEGylated recombinant staphylokinase variant, V-10153, SonoLysis Prolyse, NeuroVax, CZEN-002, rGLP-1, BIM-51077, LY-548806, exenatide (controlled release, Medisorb), AVE-0010, GA-GCB, avorelin, ACM-9604, linaclotid eacetate, CETi-1, Hemospan, VAL, fast-acting insulin (injectable, Viadel), insulin (eligen), recombinant methionyl human leptin, pitrakinra, Multikine, RG-1068, MM-093, NBI-6024, AT-001, PI-0824, Org-39141, Cpn10, talactoferrin, rEV-131, rEV-131, recombinant human insulin, RPI-78M, oprelvekin, CYT-99007 CTLA4-Ig, DTY-001, valategrast, interferon alpha-n3, IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, ALTU-135, recombinant neuraminidase, Vacc-5q, Vacc-4x, Tat Toxoid, YSPSL, CHS-13340, PTH(1-34) (Novasome), Ostabolin-C, PTH analog, MBRI-93.02, MTB72F, MVA-Ag85A, FARA04, BA-210, recombinant plague FIV, AG-702, OxSODrol, rBetV1, Der-p1/Der-p2/Der-p′7, PR1 peptide antigen, mutant ras vaccine, HPV-16 E7 lipopeptide vaccine, labyrinthin, WT1-peptide, IDD-5, CDX-110, Pentrys, Norelin, CytoFab, P-9808, VT-111, icrocaptide, telbermin, rupintrivir, reticulose, rGRF, HA, alpha-galactosidase A, ACE-011, ALTU-140, CGX-1160, angiotensin, D-4F, ETC-642, APP-018, rhMBL, SCV-07, DRF-7295, ABT-828, ErbB2-specific immunotoxin, DT3SSIL-3, TST-10088, PRO-1762, Combotox, cholecystokinin-B/gastrin-receptor binding peptides, 111In-hEGF, AE-37, trasnizumab-DM1, Antagonist G, IL-12, PM-02734, IMP-321, rhIGF-BP3, BLX-883, CUV-1647, L-19 based ra, Re-188-P-2045, AMG-386, DC/1540/KLH, VX-001, AVE-9633, AC-9301, NY-ESO-1 (peptides), NA17.A2 peptides, CBP-501, recombinant human lactoferrin, FX-06, AP-214, WAP-8294A, ACP—HIP, SUN-11031, peptide YY [3-36], FGLL, atacicept, BR3-Fc, BN-003, BA-058, human parathyroid hormone 1-34, F-18-CCR1, AT-1100, JPD-003, PTH(7-34) (Novasome), duramycin, CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix, EP-51216, hGH, OGP-I, sifuvirtide, TV4710, ALG-889, Org-41259, rhCC10, F-991, thymopentin, r(m)CRP, hepatoselective insulin, subalin, L19-IL-2 fusion protein, elafin, NMK-150, ALTU-139, EN-122004, rhTPO, thrombopoietin receptor agonist, AL-108, AL-208, nerve growth factor antagonists, SLV-317, CGX-1007, INNO-105, teriparatide (eligen), GEM-OS1, AC-162352, PRX-302, LFn-p24 fusion, EP-1043, gpE1, gpE2, MF-59, hPTH(1-34), 768974, SYN-101, PGN-0052, aviscumnine, BIM-23190, multi-epitope tyrosinase peptide, enkastim, APC-8024, GI-5005, ACC-001, TTS-CD3, vascular-targeted TNF, desmopressin, onercept, and TP-9201.

In some embodiments, the polypeptide is adalimumab (HUMIRA), infliximab (REMICADE™), rituximab (RITUXAN™/MAB THERA™) etanercept (ENBREL™) bevacizumab (AVASTIN™), trastuzumab (HERCEPTIN™), pegrilgrastim (NEULASTA™), or any other suitable polypeptide including biosimilars and biobetters.

Other suitable polypeptides are those listed below and in Table 1 of US2016/0097074:

TABLE I

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion protein | Amevive ® |
| LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta- | Ceredase ® |
| glucocerebrosidase | |
| imiglucerase; recombinant form of beta- | Cerezyme ® |
| glucocerebrosidase | |
| crotalidae polyvalent immune Fab, ovine | CroFab ™ |
| digoxin immune fab [ovine] | DigiFab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von Willebrand factor | Humate-P ® |
| complex-human | |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| eptifibatide | Integrilin ™ |
| alpha-interferon | Intron A ® |
| Palifermin | Kepivance |
| Anakinra | Kineret ™ |
| antihemophilic factor | Kogenate ®FS |
| insulin glargine | Lantus ® |
| granulocyte macrophage colony-stimulating | Leukine ®/Leukine ® Liquid |
| factor | |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly LeuTech ®) |
| somatropin [rDNA] | Norditropin ®/Norditropin Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion protein | Orencia ™ |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| peginterferon alfa-2a | Pegasys ® |
| pegylated version of interferon alfa-2b | PEG-Intron ™ |
| Abarelix (injectable suspension); gonadotropin-releasing | Plenaxis ™ |
| hormone antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective, reversible T-cell blocker | RAPTIVA ™ |
| combination of ribavirin and alpha interferon | Rebetron ™ |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Eculizumab | SOLIRIS (R) |
| Pegvisomant | SOMAVERT ® |
| Palivizumab; recombinantly produced, humanized mAb | Synagis ™ |
| thyrotropin alfa | Thyrogen ® |
| Tenecteplase | TNKase ™ |
| Natalizumab | TYSABRI ® |
| human immune globulin intravenous 5% and 10% solutions | Venoglobulin-S ® |
| interferon alfa-n1, lymphoblastoid | Wellferon ® |
| drotrecogin alfa | Xigris ™ |
| Omalizumab; recombinant DNA-derived humanized monoclonal | Xolair ® |
| antibody targeting immunoglobulin-E | |
| Daclizumab | Zenapax ® |
| Ibritumomab tiuxetan | Zevalin ™ |
| Somatotropin | Zorbtive ™ (Serostim ®) |

In embodiments, the polypeptide is a hormone, blood clotting/coagulation factor, cytokine/growth factor, antibody molelcule, fusion protein, protein vaccine, or peptide as shown in Table 2.

TABLE 2
Exemplary Products

| Therapeutic | | |
|---|---|---|
| Product type | Product | Trade Name |
| Hormone | Erythropoietin, Epoein-α | Epogen, Procrit |
| | Darbepoetin-α | Aranesp |
| | Growth hormone (GH), | Genotropin, Humatrope, Norditropin, |
| | somatotropin | NovIVitropin, Nutropin, Omnitrope, |
| | | Protropin, Siazen, Serostim, Valtropin |
| | Human follicle-stimulating | Gonal-F, Follistim |
| | hormone (FSH) | |
| | Human chorionic | Ovidrel |
| | gonadotropin | |
| | Lutropin-α | Luveris |
| | Glucagon | GlcaGen |
| | Growth hormone releasing | Geref |
| | hormone (GHRH) | |
| | Secretin | ChiRhoStim (human peptide), SecreFlo |
| | | (porcine peptide) |
| | Thyroid stimulating | Thyrogen |
| | hormone (TSH), thyrotropin | |
| Blood | Factor VIIa | NovoSeven |
| Clotting/Coagulation | Factor VIII | Bioclate, Helixate, Kogenate, |
| Factors | | Recombinate, ReFacto |
| | Factor IX | Benefix |
| | Antithrombin III (AT-III) | Thrombate III |
| | Protein C concentrate | Ceprotin |
| Cytokine/Growth | Type I alpha-interferon | Infergen |
| factor | Interferon-αn3 (IFNαn3) | Alferon N |
| | Interferon-β1a (rIFN- β) | Avonex, Rebif |
| | Interferon-β1b (rIFN- β) | Betaseron |
| | Interferon-γ1b (IFN γ) | Actimmune |
| | Aldesleukin (interleukin | Proleukin |
| | 2(IL2), epidermal | |
| | theymocyte activating | |
| | factor; ETAF | |
| | Palifermin (keratinocyte | Kepivance |
| | growth factor; KGF) | |
| | Becaplemin (platelet- | Regranex |
| | derived growth factor; | |
| | PDGF) | |
| | Anakinra (recombinant IL1 | Anril, Kineret |
| | antagonist) | |
| Antibody molecules | Bevacizumab (VEGFA | Avastin |
| | mAb) | |
| | Cetuximab (EGFR mAb) | Erbitux |
| | Panitumumab (EGFR mAb) | Vectibix |
| | Alemtuzumab (CD52 mAb) | Campath |
| | Rituximab (CD20 chimeric | Rituxan |
| | Ab) | |
| | Trastuzumab (HER2/Neu | Herceptin |
| | mAb) | |
| | Abatacept (CTLA Ab/Fc | Orencia |
| | fusion) | |
| | Adalimumab (TNFα mAb) | Humira |
| | Etanercept (TNF | Enbrel |
| | receptor/Fc fusion) | |
| | Infliximab (TNFα chimeric | Remicade |
| | mAb) | |
| | Alefacept (CD2 fusion | Amevive |
| | protein) | |
| | Efalizumab (CD11a mAb) | Raptiva |
| | Natalizumab (integrin α4 | Tysabri |
| | subunit mAb) | |
| | Eculizumab (C5mAb) | Soliris |
| | Muromonab-CD3 | Orthoclone, OKT3 |
| Other: | Insulin | Humulin, Novolin |
| Fusion | Hepatitis B surface antigen | Engerix, Recombivax HB |
| proteins/Protein | (HBsAg) | |
| vaccines/Peptides | HPV vaccine | Gardasil |
| | OspA | LYMErix |
| | Anti-Rhesus(Rh) | Rhophylac |
| | immunoglobulin G | |
| | Enfuvirtide | Fuzeon |
| | Spider silk, e.g., fibrion | QMONOS |

In embodiments, the protein is a multispecific protein, e.g., a bispecific antibody as shown in Table 3.

TABLE 3
Bispecific Formats

| Name (other | | | | | |
|---|---|---|---|---|---|
| names, | | | Proposed | | Diseases (or |
| sponsoring | BsAb | | mechanisms of | Development | healthy |
| organizations) | format | Targets | action | stages | volunteers) |
| Catumaxomab | BsIgG: | CD3, | Retargeting of T | Approved in | Malignant ascites |
| (Removab ®, | Triomab | EpCAM | cells to tumor, Fc | EU | in EpCAM |
| Fresenius Biotech, | | | mediated effector | | positive tumors |
| Trion Pharma, | | | functions | | |
| Neopharm) | | | | | |
| Ertumaxomab | BsIgG: | CD3, HER2 | Retargeting of T | Phase I/II | Advanced solid |
| (Neovii Biotech, | Triomab | | cells to tumor | | tumors |
| Fresenius Biotech) | | | | | |
| Blinatumomab | BiTE | CD3, CD19 | Retargeting of T | Approved in | Precursor B-cell |
| (Blincyto ®, AMG | | | cells to tumor | USA | ALL |
| 103, MT 103, | | | | Phase II and | ALL |
| MEDI 538, | | | | III | DLBCL |
| Amgen) | | | | Phase II | NHL |
| | | | | Phase I | |
| REGN1979 | BsAb | CD3, CD20 | | | |
| (Regeneron) | | | | | |
| Solitomab (AMG | BiTE | CD3, | Retargeting of T | Phase I | Solid tumors |
| 110, MT110, | | EpCAM | cells to tumor | | |
| Amgen) | | | | | |
| MEDI 565 (AMG | BiTE | CD3, CEA | Retargeting of T | Phase I | Gastrointestinal |
| 211, MedImmune, | | | cells to tumor | | adenocancinoma |
| Amgen) | | | | | |
| RO6958688 | BsAb | CD3, CEA | | | |
| (Roche) | | | | | |
| BAY2010112 | BiTE | CD3, PSMA | Retargeting of T | Phase I | Prostate cancer |
| (AMG 212, Bayer; | | | cells to tumor | | |
| Amgen) | | | | | |
| MGD006 | DART | CD3, CD123 | Retargeting of T | Phase I | AML |
| (Macrogenics) | | | cells to tumor | | |
| MGD007 | DART | CD3, gpA33 | Retargeting of T | Phase I | Colorectal cancer |
| (Macrogenics) | | | cells to tumor | | |
| MGD011 | DART | CD19, CD3 | | | |
| (Macrogenics) | | | | | |
| SCORPION | BsAb | CD3, CD19 | Retargeting of T | | |
| (Emergent | | | cells to tumor | | |
| Biosolutions, | | | | | |
| Trubion) | | | | | |
| AFM11 (Affimed | TandAb | CD3, CD19 | Retargeting of T | Phase I | NHL and ALL |
| Therapeutics) | | | cells to tumor | | |
| AFM12 (Affimed | TandAb | CD19, CD16 | Retargeting of NK | | |
| Therapeutics) | | | cells to tumor | | |
| | | | cells | | |
| AFM13 (Affimed | TandAb | CD30, | Retargeting of NK | Phase II | Hodgkin's |
| Therapeutics) | | CD16A | cells to tumor | | Lymphoma |
| | | | cells | | |
| GD2 (Barbara Ann | T cells | CD3, GD2 | Retargeting of T | Phase I/II | Neuroblastoma |
| Karmanos Cancer | preloaded | | cells to tumor | | and |
| Institute) | with BsAb | | | | osteosarcoma |
| pGD2 (Barbara | T cells | CD3, Her2 | Retargeting of T | Phase II | Metastatic breast |
| Ann Karmanos | preloaded | | cells to tumor | | cancer |
| Cancer Institute) | with BsAb | | | | |
| EGFRBi-armed | T cells | CD3, EGFR | Autologous | Phase I | Lung and other |
| autologous | preloaded | | activated T cells | | solid tumors |
| activated T cells | with BsAb | | to EGFR-positive | | |
| (Roger Williams | | | tumor | | |
| Medical Center) | | | | | |
| Anti-EGFR-armed | T cells | CD3, EGFR | Autologous | Phase I | Colon and |
| activated T-cells | preloaded | | activated T cells | | pancreatic |
| (Barbara Ann | with BsAb | | to EGFR-positive | | cancers |
| Karmanos Cancer | | | tumor | | |
| Institute) | | | | | |
| rM28 (University | Tandem | CD28, | Retargeting of T | Phase II | Metastatic |
| Hospital Tübingen) | scFv | MAPG | cells to tumor | | melanoma |
| IMCgp100 | ImmTAC | CD3, peptide | Retargeting of T | Phase I/II | Metastatic |
| (Immunocore) | | MHC | cells to tumor | | melanoma |
| DT2219ARL | 2 scFv | CD19, CD22 | Targeting of | Phase I | B cell leukemia |
| (NCI, University of | linked to | | protein toxin to | | or lymphoma |
| Minnesota) | diphtheria | | tumor | | |
| | toxin | | | | |
| XmAb5871 | BsAb | CD19, | | | |
| (Xencor) | | CD32b | | | |
| NI-1701 | BsAb | CD47, CD19 | | | |
| (NovImmune) | | | | | |
| MM-111 | BsAb | ErbB2, | | | |
| (Merrimack) | | ErbB3 | | | |
| MM-141 | BsAb | IGF-1R, | | | |
| (Merrimack) | | ErbB3 | | | |
| NA (Merus) | BsAb | HER2, | | | |
| | | HER3 | | | |
| NA (Merus) | BsAb | CD3, | | | |
| | | CLEC12A | | | |
| NA (Merus) | BsAb | EGFR, | | | |
| | | HER3 | | | |
| NA (Merus) | BsAb | PD1, | | | |
| | | undisclosed | | | |
| NA (Merus) | BsAb | CD3, | | | |
| | | undisclosed | | | |
| Duligotuzumab | DAF | EGFR, | Blockade of 2 | Phase I and II | Head and neck |
| (MEHD7945A, | | HER3 | receptors, ADCC | Phase II | cancer |
| Genentech, Roche) | | | | | Colorectal cancer |
| LY3164530 (Eli | Not | EGFR, MET | Blockade of 2 | Phase I | Advanced or |
| Lily) | disclosed | | receptors | | metastatic cancer |
| MM-111 | HSA body | HER2, | Blockade of 2 | Phase II | Gastric and |
| (Merrimack | | HER3 | receptors | Phase I | esophageal |
| Pharmaceuticals) | | | | | cancers |
| | | | | | Breast cancer |
| MM-141, | IgG-scFv | IGF-1R, | Blockade of 2 | Phase I | Advanced solid |
| (Merrimack | | HER3 | receptors | | tumors |
| Pharmaceuticals) | | | | | |
| RG7221 | CrossMab | Ang2, VEGFA | Blockade of 2 | Phase I | Solid tumors |
| (RO5520985, | | | proangiogenics | | |
| Roche) | | | | | |
| RG7716 (Roche) | CrossMab | Ang2, VEGFA | Blockade of 2 | Phase I | Wet AMD |
| | | | proangiogenics | | |
| OMP-305B83 | BsAb | DLL4/VEGF | | | |
| (OncoMed) | | | | | |
| TF2 | Dock and | CEA, HSG | Pretargeting | Phase II | Colorectal, |
| (Immunomedics) | lock | | tumor for PET or | | breast and lung |
| | | | radioimaging | | cancers |
| ABT-981 | DVD-Ig | IL-1α, IL-1β | Blockade of 2 | Phase II | Osteoarthritis |
| (AbbVie) | | | proinflammatory | | |
| | | | cytokines | | |
| ABT-122 | DVD-Ig | TNF, IL-17A | Blockade of 2 | Phase II | Rheumatoid |
| (AbbVie) | | | proinflammatory | | arthritis |
| | | | cytokines | | |
| COVA322 | IgG-fynomer | TNF, IL17A | Blockade of 2 | Phase I/II | Plaque psoriasis |
| | | | proinflammatory | | |
| | | | cytokines | | |
| SAR156597 | Tetravalent | IL-13, IL-4 | Blockade of 2 | Phase I | Idiopathic |
| (Sanofi) | bispecific | | proinflammatory | | pulmonary |
| | tandem IgG | | cytokines | | fibrosis |
| GSK2434735 | Dual- | IL-13, IL-4 | Blockade of 2 | Phase I | (Healthy |
| (GSK) | targeting | | proinflammatory | | volunteers) |
| | domain | | cytokines | | |
| Ozoralizumab | Nanobody | TNF, HSA | Blockade of | Phase II | Rheumatoid |
| (ATN103, Ablynx) | | | proinflammatory | | arthritis |
| | | | cytokine, binds to | | |
| | | | HSA to increase | | |
| | | | half-life | | |
| ALX-0761 (Merck | Nanobody | IL-17A/F, | Blockade of 2 | Phase I | (Healthy |
| Serono, Ablynx) | | HSA | proinflammatory | | volunteers) |
| | | | cytokines, binds | | |
| | | | to HSA to | | |
| | | | increase half-life | | |
| ALX-0061 | Nanobody | IL-6R, HSA | Blockade of | Phase I/II | Rheumatoid |
| (AbbVie, Ablynx; | | | proinflammatory | | arthritis |
| | | | cytokine, binds to | | |
| | | | HSA to increase | | |
| | | | half-life | | |
| ALX-0141 | Nanobody | RANKL, | Blockade of bone | Phase I | Postmenopausal |
| (Ablynx, | | HSA | resorption, binds | | bone loss |
| Eddingpharm) | | | to HSA to | | |
| | | | increase half-life | | |
| RG6013/ACE910 | ART-Ig | Factor IXa, | Plasma | Phase II | Hemophilia |
| (Chugai, Roche) | | factor X | coagulation | | |

TABLE 4

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| Interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion protein | Amevive ® |
| LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta- | Ceredase ® |
| glucocerebrosidase | |
| imiglucerase; recombinant form of beta- | Cerezyme ® |
| glucocerebrosidase | |
| crotalidae polyvalent immune Fab, ovine | CroFab ™ |
| digoxin immune fab [ovine] | DigiFab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von Willebrand factor | Humate-P ® |
| complex-human | |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| Eptifibatide | Integrilin ™ |
| alpha-interferon | Intron A ® |
| Palifermin | Kepivance |
| Anakinra | Kineret ™ |
| antihemophilic factor | Kogenate ® FS |
| insulin glargine | Lantus ® |
| granulocyte macrophage colony-stimulating | Leukine ®/Leukine ® Liquid |
| factor | |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly LeuTech ®) |
| somatropin [rDNA] | Norditropin ®/Norditropin Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion protein | Orencia ™ |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| peginterferon alfa-2a | Pegasys ® |
| pegylated version of interferon alfa-2b | PEG-Intron ™ |
| Abarelix (injectable suspension); gonadotropin-releasing | Plenaxis ™ |
| hormone | |
| Antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective, reversible T-cell blocker | RAPTIVA ™ |
| combination of ribavirin and alpha interferon | Rebetron ™ |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Eculizumab | SOLIRIS (R) |
| Pegvisomant | SOMAVERT ® |
| Palivizumab; recombinantly produced, humanized mAb | Synagis ™ |
| thyrotropin alfa | Thyrogen ® |
| Tenecteplase | TNKase ™ |
| Natalizumab | TYSABRI ® |
| human immune globulin intravenous 5% and 10% solutions | Venoglobulin-S ® |
| interferon alfa-n1, lymphoblastoid | Wellferon ® |
| drotrecogin alfa | Xigris ™ |
| Omalizumab; recombinant DNA-derived humanized monoclonal | Xolair ® |
| antibody targeting immunoglobulin-E | |
| Daclizumab | Zenapax ® |
| ibritumomab tiuxetan | Zevalin ™ |
| Somatotropin | Zorbtive ™ (Serostim ®) |

In some embodiments, the polypeptide is an antigen expressed by a cancer cell. In some embodiments the recombinant or therapeutic polypeptide is a tumor-associated antigen or a tumor-specific antigen. In some embodiments, the recombinant or therapeutic polypeptide is selected from HER2, CD20, 9-O-acetyl-GD3, βhCG, A33 antigen, CA19-9 marker, CA-125 marker, calreticulin, carboanhydrase IX (MN/CA IX), CCR5, CCR8, CD19, CD22, CD25, CD27, CD30, CD33, CD38, CD44v6, CD63, CD70, CC123, CD138, carcinoma embryonic antigen (CEA; CD66e), desmoglein 4, E-cadherin neoepitope, endosialin, ephrin A2 (EphA2), epidermal growth factor receptor (EGFR), epithelial cell adhesion molecule (EpCAM), ErbB2, fetal acetylcholine receptor, fibroblast activation antigen (FAP), fucosyl GM1, GD2, GD3, GM2, ganglioside GD3, Globo H, glycoprotein 100, HER2/neu, HER3, HER4, insulin-like growth factor receptor 1, Lewis-Y, LG, Ly-6, melanoma-specific chondroitin-sulfate proteoglycan (MCSCP), mesothelin, MUC1, MUC2, MUC3, MUC4, MUC5_{AC}, MUCS_{B}, MUC7, MUC16, Mullerian inhibitory substance (MIS) receptor type II, plasma cell antigen, poly SA, PSCA, PSMA, sonic hedgehog (SHH), SAS, STEAP, sTn antigen, TNF-alpha precursor, and combinations thereof.

In some embodiments, the polypeptide is an activating receptor and is selected from 2B4 (CD244), α₄β₁ integrin, β₂ integrins, CD2, CD16, CD27, CD38, CD96, CD100, CD160, CD137, CEACAM1(CD66), CRTAM, CS1 (CD319), DNAM-1 (CD226), GITR (TNFRSF18), activating forms of KIR, NKG2C, NKG2D, NKG2E, one or more natural cytotoxicity receptors, NTB-A, PEN-5, and combinations thereof, optionally wherein the β₂ integrins comprise CD11a-CD 18, CD11 b-CD 18, or CD11c-CD 18, optionally wherein the activating forms of KIR comprise K1R2DS1, KIR2DS4, or KIR-S, and optionally wherein the natural cytotoxicity receptors comprise NKp30, NKp44, NKp46, or NKp80.

In some embodiments, the polypeptide is an inhibitory receptor and is selected from KIR, ILT2/LIR-1/CD85j, inhibitory forms of KIR, KLRG1, LAIR-1, NKG2A, NKR-P1A, Siglec-3, Siglec-7, Siglec-9, and combinations thereof, optionally wherein the inhibitory forms of KIR comprise KIR2DL1, KIR2DL2, KIR2DL3, KIR3DL1, KIR3DL2, or KIR-L.

In some embodiments, the polypeptide is an activating receptor and is selected from CD3, CD2 (LFA2, OX34), CD5, CD27 (TNFRSF7), CD28, CD30 (TNFRSF8), CD40L, CD84 (SLAMF5), CD137 (4-1BB), CD226, CD229 (Ly9, SLAMF3), CD244 (2B4, SLAMF4), CD319 (CRACC, BLAME), CD352 (Ly108, NTBA, SLAMF6), CRTAM (CD355), DR3 (TNFRSF25), GITR (CD357), HVEM (CD270), ICOS, LIGHT, LTβR (TNFRSF3), OX40 (CD134), NKG2D, SLAM (CD150, SLAMF1), TCRα, TCRβ, TCRδγ, TIM1 (HAVCR, KIM1), and combinations thereof.

In some embodiments, the polypeptide is an inhibitory receptor and is selected from PD-1 (CD279), 2B4 (CD244, SLAMF4), B71 (CD80), B7H1 (CD274, PD-L1), BTLA (CD272), CD160 (BY55, NK28), CD352 (Ly108, NTBA, SLAMF6), CD358 (DR6), CTLA-4 (CD152), LAG3, LAIR1, PD-1H (VISTA), TIGIT (VSIG9, VSTM3), TIM2 (TIMD2), TIM3 (HAVCR2, KIM3), and combinations thereof.

Other exemplary proteins include, but are not limited to any protein described in Tables 1-10 of Leader et al., “Protein therapeutics: a summary and pharmacological classification”, Nature Reviews Drug Discovery, 2008, 7:21-39 (incorporated herein by reference); or any conjugate, variant, analog, or functional fragment of the recombinant polypeptides described herein.

Other recombinant protein products include non-antibody scaffolds or alternative protein scaffolds, such as, but not limited to: DARPins, affibodies and adnectins. Such non-antibody scaffolds or alternative protein scaffolds can be engineered to recognize or bind to one or two, or more, e.g., 1, 2, 3, 4, or 5 or more, different targets or antigens.

Also provided herein are nucleic acids, e.g., exogenous nucleic acids that encode the products, e.g., polypeptides, e.g., recombinant polypeptides described herein. The nucleic acid sequences coding for the desired recombinant polypeptides can be obtained using recombinant methods known in the art, such as, for example by screening libraries from cells expressing the desired nucleic acid sequence, e.g., gene, by deriving the nucleic acid sequence from a vector known to include the same, or by isolating directly from cells and tissues containing the same, using standard techniques. Alternatively, the nucleic acid encoding the recombinant polypeptide can be produced synthetically, rather than cloned. Recombinant DNA techniques and technology are highly advanced and well established in the art. Accordingly, the ordinarily skilled artisan having the knowledge of the amino acid sequence of a recombinant polypeptide described herein can readily envision or generate the nucleic acid sequence that would encode the recombinant polypeptide.

In some embodiments, the exogenous nucleic acid controls the expression of a product that is endogenously expressed by the host cell. In such embodiments, the exogenous nucleic acid comprises one or more nucleic acid sequences that increase the expression of the endogenous product (also referred to herein as “endogenous product transactivation sequence”). For example, the nucleic acid sequence that increases the expression of an endogenous product comprises a constitutively active promoter or a promoter that is stronger, e.g., increases transcription at the desired site, e.g., increases expression of the desired endogenous gene product. After introduction of the exogenous nucleic acid comprising the endogenous product transactivation sequence, said exogenous nucleic acid is integrated into the chromosomal genome of the cell, e.g., at a preselected location proximal to the genomic sequence encoding the endogenous product, such that the endogenous product transactivation sequence increases the transactivation or expression of the desired endogenous product. Other methods for modifying a cell, e.g., introducing an exogenous nucleic acid, for increasing expression of an endogenous product is described, e.g., in U.S. Pat. No. 5,272,071; hereby incorporated by reference in its entirety.

The expression of a product described herein is typically achieved by operably linking a nucleic acid encoding the recombinant polypeptide or portions thereof to a promoter, and incorporating the construct into an expression vector. The vectors can be suitable for replication and integration eukaryotes or prokaryotes. Typical cloning vectors contain other regulatory elements, such as transcription and translation terminators, initiation sequences, and promoters useful for regulation of the expression of the desired nucleic acid sequence.

The nucleic acid sequences described herein encoding a product, e.g., a recombinant polypeptide, or comprising a nucleic acid sequence that can control the expression of an endogenous product, can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In embodiments, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193). Vectors derived from viruses are suitable tools to achieve long-term gene transfer since they allow long-term, stable integration of a transgene and its propagation in daughter cells.

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection, e.g., a selection marker or a reporter gene.

In one embodiment, the vector comprising a nucleic acid sequence encoding a polypeptide, e.g., a recombinant polypeptide, further comprises a promoter sequence responsible for the recruitment of polymerase to enable transcription initiation for expression of the polypeptide, e.g., the recombinant polypeptide. In one embodiment, promoter sequences suitable for the methods described herein are usually associated with enhancers to drive high amounts of transcription and hence deliver large copies of the target exogenous mRNA. In an embodiment, the promoter comprises cytomegalovirus (CMV) major immediate early promoters (Xia, Bringmann et al. 2006) and the SV40 promoter (Chernajovsky, Mory et al. 1984), both derived from their namesake viruses or promoters derived therefrom. Several other less common viral promoters have been successfully employed to drive transcription upon inclusion in an expression vector including Rous Sarcoma virus long terminal repeat (RSV-LTR) and Moloney murine leukaemia virus (MoMLV) LTR (Papadakis, Nicklin et al. 2004). In another embodiment, specific endogenous mammalian promoters can be utilized to drive constitutive transcription of a gene of interest (Pontiller, Gross et al. 2008). The CHO specific Chinese Hamster elongation factor 1-alpha (CHEF1a) promoter has provided a high yielding alternative to viral based sequences (Deer, Allison 2004). In addition to promoters, the vectors described herein further comprise an enhancer region as described above; a specific nucleotide motif region, proximal to the core promoter, which can recruit transcription factors to upregulate the rate of transcription (Riethoven 2010). Similar to promoter sequences, these regions are often derived from viruses and are encompassed within the promoter sequence such as hCMV and SV40 enhancer sequences, or may be additionally included such as adenovirus derived sequences (Gaillet, Gilbert et al. 2007).

In one embodiment, the vector comprising a nucleic acid sequence encoding a product, e.g., a polypeptide, e.g, a recombinant polypeptide, described herein further comprises a nucleic acid sequence that encodes a selection marker. In one embodiment, the selectable marker comprises glutamine synthetase (GS); dihydrofolate reductase (DHFR) e.g., an enzyme which confers resistance to methotrexate (MTX); or an antibiotic marker, e.g., an enzyme that confers resistance to an antibiotic such as: hygromycin, neomycin (G418), zeocin, puromycin, or blasticidin. In another embodiment, the selection marker comprises or is compatible with the Selexis selection system (e.g., SUREtechnology Platform™ and Selexis Genetic Elements™ commercially available from Selexis SA) or the Catalant selection system.

In one embodiment, the vector comprising a nucleic acid sequence encoding a recombinant product described herein comprises a selection marker that is useful in identifying a cell or cells comprise the nucleic acid encoding a recombinant product described herein. In another embodiment, the selection marker is useful in identifying a cell or cells that comprise the integration of the nucleic acid sequence encoding the recombinant product into the genome, as described herein. The identification of a cell or cells that have integrated the nucleic acid sequence encoding the recombinant protein can be useful for the selection and engineering of a cell or cell line that stably expresses the product.

Suitable vectors for use are commercially available, and include vectors associated with the GS Expression System™, GS Xceed™ Gene Expression System, or Potelligent® CHOK1SV technology available from Lonza Biologics, Inc, e.g., vectors as described in Fan et al., Pharm. Bioprocess. (2013); 1(5):487-502, which is incorporated herein by reference in its entirety. GS expression vectors comprise the GS gene, or a functional fragment thereof (e.g., a GS mini-gene), and one or more, e.g., 1, 2, or 3, or more, highly efficient transcription cassettes for expression of the gene of interest, e.g., a nucleic acid encoding a recombinant polypeptide described herein. A GS mini-gene comprises, e.g., consists of, intron 6 of the genomic CHO GS gene. In one embodiment, a GS vector comprises a GS gene operably linked to a SV40L promoter and one or two polyA signals. In another embodiment, a GS vector comprises a GS gene operably linked to a SV40E promoter, SV40 splicing and polyadenylation signals. In such embodiments, the transcription cassette, e.g., for expression of the gene of interest or recombinant polypeptide described herein, includes the hCMV-MIE promoter and 5′ untranslated sequences from the hCMV-MIE gene including the first intron. Other vectors can be constructed based on GS expression vectors, e.g., wherein other selection markers are substituted for the GS gene in the expression vectors described herein.

Vectors suitable for use in the methods described herein include, but are not limited to, other commercially available vectors, such as, pcDNA3.1/Zeo, pcDNA3.1/CAT, pcDNA3.3TOPO (Thermo Fisher, previously Invitrogen); pTarget, HaloTag (Promega); pUC57 (GenScript); pFLAG-CMV (Sigma-Aldrich); pCMV6 (Origene); pEE12 or pEE14 (Lonza Biologics), or pBK-CMV/pCMV-3Tag-7/pCMV-Tag2B (Stratagene).

### Cells and Cell Culture

In embodiments, the cell is a mammalian cell. In other embodiments, the cell is a cell other than a mammalian cell. In an embodiment, the cell is a mouse, rat, Chinese hamster, Syrian hamster, monkey, ape, dog, horse, ferret, or cat. In embodiments, the cell is a mammalian cell, e.g., a human cell or a rodent cell, e.g., a hamster cell, a mouse cell, or a rat cell. In another embodiment, the cell is from a duck, parrot, fish, insect, plant, fungus, or yeast. In one embodiment, the cell is an Archaebacteria. In an embodiment, the cell is a species of Actinobacteria, e.g., Mycobacterium tuberculosis).

In one embodiment, the cell is a Chinese hamster ovary (CHO) cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1SV GS knockout cell (Lonza Biologics, Inc.). The CHO FUT8 knockout cell is, for example, the Potelligent® CHOK1 SV (Lonza Biologics, Inc.).

In another embodiment, the cell is a Hela, HEK293, HT1080, H9, HepG2, MCF7, Jurkat, NIH3T3, PC12, PER.C6, BHK (baby hamster kidney cell), VERO, SP2/0, NS0, YB2/0, Y0, EB66, C127, L cell, COS, e.g., COS1 and COST, QC1-3, CHOK1, CHOK1SV, Potelligent CHOK1SV, CHO GS knockout, CHOK1SV GS-KO, CHOS, CHO DG44, CHO DXB11, and CHOZN, or any cells derived therefrom. In one embodiment, the cell is a stem cell. In one embodiment, the cell is a differentiated form of any of the cells described herein. In one embodiment, the cell is a cell derived from any primary cell in culture.

In an embodiment, the cell is any one of the cells described herein that comprises an exogenous nucleic acid encoding a recombinant polypeptide, e.g., expresses a recombinant polypeptide, e.g., a recombinant polypeptide selected from Table 1 or 2.

### Large Scale Production

The devices, facilities and methods described herein are suitable for culturing any desired cell line including prokaryotic and/or eukaryotic cell lines. Further, in embodiments, the devices, facilities and methods are suitable for culturing suspension cells or anchorage-dependent (adherent) cells and are suitable for production operations configured for production of pharmaceutical and biopharmaceutical products—such as polypeptide products, nucleic acid products (for example DNA or RNA), or cells and/or viruses such as those used in cellular and/or viral therapies.

In embodiments, the cells express or produce a product, such as a recombinant therapeutic or diagnostic product. Examples of products produced by cells include, but are not limited to, antibody molecules (e.g., monoclonal antibodies, bispecific antibodies), antibody mimetics (polypeptide molecules that bind specifically to antigens but that are not structurally related to antibodies such as e.g. DARPins, affibodies, adnectins, or IgNARs), fusion proteins (e.g., Fc fusion proteins, chimeric cytokines), other recombinant proteins (e.g., glycosylated proteins, enzymes, hormones), viral therapeutics (e.g., anti-cancer oncolytic viruses, viral vectors for gene therapy and viral immunotherapy), cell therapeutics (e.g., pluripotent stem cells, mesenchymal stem cells and adult stem cells), vaccines or lipid-encapsulated particles (e.g., exosomes, virus-like particles), RNA (such as e.g. siRNA) or DNA (such as e.g. plasmid DNA), antibiotics or amino acids. In embodiments, the devices, facilities and methods can be used for producing biosimilars.

As mentioned, in embodiments, devices, facilities and methods allow for the production of eukaryotic cells, e.g., mammalian cells or lower eukaryotic cells such as for example yeast cells or filamentous fungi cells, or prokaryotic cells such as Gram-positive or Gram-negative cells and/or products of the eukaryotic or prokaryotic cells, e.g., proteins, peptides, antibiotics, amino acids, nucleic acids (such as DNA or RNA), synthesised by the eukaryotic cells in a large-scale manner. Unless stated otherwise herein, the devices, facilities, and methods can include any desired volume or production capacity including but not limited to bench-scale, pilot-scale, and full production scale capacities.

In embodiments, devices and methods allow for the production of cells and products of the cells, especially proteins, peptides (discussed in detail above), antibiotics or amino acids, synthesized by cells, e.g., mammalian cells, in a large-scale manner.

A wide array of flasks, bottles, reactors, and controllers allow the production and scale up of cell culture systems. The system can be chosen based, at least in part, upon its correlation with a desired glycan property or properties. Cells can be grown, for example, as batch, fed-batch, perfusion, or continuous cultures. Production parameters that can be selected include, e.g., addition or removal of media including when (early, middle or late during culture time) and how often media is harvested; increasing or decreasing speed at which cell cultures are agitated; increasing or decreasing temperature at which cells are cultured; adding or removing media such that culture density is adjusted; selecting a time at which cell cultures are started or stopped; and selecting a time at which cell culture parameters are changed. Such parameters can be selected for any of the batch, fed-batch, perfusion and continuous culture conditions.

In embodiments, the cultivated cells for large scale production are eukaryotic cells, e.g., animal cells, e.g., mammalian cells. The mammalian cells can be, for example, human cell lines, mouse myeloma (NSO)-cell lines, Chinese hamster ovary (CHO)-cell lines or hybri-doma-cell lines. Preferably the mammalian cells are CHO-cell lines.

In embodiments, the cultivated cells for large scale production re used to produce antibodies discussed in detail above, e.g., monoclonal antibodies, and/or recombinant proteins, e.g., recombinant proteins for therapeutic use. In embodiments, the cells produce peptides, amino acids, fatty acids or other useful biochemical intermediates or metabolites.

In embodiments, the cells for large scale production are eukaryotic cells, biochemical markers, recombinant peptides or nucleotide sequences of interest, proteins, yeast, insect cells, stable or viral infected, avian cells or mammalian cells such as CHO cells, monkey cells, lytic products and the like for medical, research or commercial purposes.

In embodiments, the cells for large scale production are prokaryotic cells, strains of Gram-positive cells such as Bacillus and Streptomyces. In embodiments, the host cell is of phylum Firmicutes, e.g., the host cell is Bacillus. BS acillus that can be used are, e.g. the strains B. subtilis, B. amyloliquefaciens, B. licheniformis, B. natto, B. megaterium, etc. In embodiments, the host cell is B. subtilis, such as B. subtilis 3NA and B. subtilis 168. Bacillus is obtainable from, e.g., the Bacillus Genetic Stock Center, Biological Sciences 556, 484 West 12^{th} Avenue, Columbus Ohio 43210-1214.

In embodiments, the prokaryotic cells for large scale production are Gram negative cells, such as Salmonella spp. or E. coli, e.g., the strains TG1, W3110, DH1, XL1-Blue and Origami, which are commercially available.

Suitable host cells are commercially available, for example, from culture collections such as the DSMZ (Deutsche Sammlung von Mikroorganismen and Zellkulturen GmbH, Braunschweig, Germany).

In an embodiment, the cell culture is carried out as a batch culture, fed-batch culture, draw and fill culture, or a continuous culture. In an embodiment, the cell culture is a suspension culture. In one embodiment, the cell or cell culture is placed in vivo for expression of the recombinant polypeptide, e.g., placed in a model organism or a human subject.

In one embodiment, the culture media is free of serum. Serum-free and protein-free media are commercially available, e.g., Lonza Biologics.

Suitable media and culture methods for mammalian cell lines are well-known in the art, as described in U.S. Pat. No. 5,633,162, for instance. Examples of standard cell culture media for laboratory flask or low density cell culture and being adapted to the needs of particular cell types are for instance: Roswell Park Memorial Institute (RPMI) 1640 medium (Morre, G., The Journal of the American Medical Association, 199, p. 519 f. 1967), L-15 medium (Leibovitz, A. et al., Amer. J. of Hygiene, 78, 1p. 173 ff, 1963), Dulbecco's modified Eagle's medium (DMEM), Eagle's minimal essential medium (MEM), Ham's F12 medium (Ham, R. et al., Proc. Natl. Acad. Sc.53, p288 ff. 1965) or Iscoves' modified DMEM lacking albumin, transferrin and lecithin (Iscoves et al., J. Exp. med. 1, p. 923 ff., 1978). For instance, Ham's F10 or F12 media were specially designed for CHO cell culture. Other media specially adapted to CHO cell culture are described in EP-481 791. It is known that such culture media can be supplemented with fetal bovine serum (FBS, also called fetal calf serum FCS), the latter providing a natural source of a plethora of hormones and growth factors. The cell culture of mammalian cells is nowadays a routine operation well-described in scientific textbooks and manuals, it is covered in detail e.g. in R. Ian Fresney, Culture of Animal cells, a manual, 4^{th} edition, Wiley-Liss/N.Y., 2000.

Other suitable cultivation methods are known to the skilled artisan and may depend upon the recombinant polypeptide product and the host cell utilized. It is within the skill of an ordinarily skilled artisan to determine or optimize conditions suitable for the expression and production of the recombinant polypeptide to be expressed by the cell.

In one aspect, the cell or cell line for large scale production comprises an exogenous nucleic acid that encodes a product, e.g., a recombinant polypeptide. In an embodiment, the cell or cell line expresses the product, e.g., a therapeutic or diagnostic product. Methods for genetically modifying or engineering a cell to express a desired polypeptide or protein are well known in the art, and include, for example, transfection, transduction (e.g., viral transduction), or electroporation.

Physical methods for introducing a nucleic acid, e.g., an exogenous nucleic acid or vector described herein, into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY).

Chemical means for introducing a nucleic acid, e.g., an exogenous nucleic acid or vector described herein, into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In embodiments, the integration of the exogenous nucleic acid into a nucleic acid of the host cell, e.g., the genome or chromosomal nucleic acid of the host cell is desired. Methods for determining whether integration of an exogenous nucleic acid into the genome of the host cell has occurred can include a GS/MSX selection method. The GS/MSX selection method uses complementation of a glutamine auxotrophy by a recombinant GS gene to select for high-level expression of proteins from cells. Briefly, the GS/MSX selection method comprises inclusion of a nucleic acid encoding glutamine synthetase on the vector comprising the exogenous nucleic acid encoding the recombinant polypeptide product. Administration of methionine sulfoximine (MSX) selects cells that have stably integrated into the genome the exogenous nucleic acid encoding both the recombinant polypeptide and GS. As GS can be endogenously expressed by some host cells, e.g., CHO cells, the concentration and duration of selection with MSX can be optimized to identify high producing cells with stable integration of the exogenous nucleic acid encoding the recombinant polypeptide product into the host genome. The GS selection and systems thereof is further described in Fan et al., Pharm. Bioprocess. (2013); 1(5):487-502, which is incorporated herein by reference in its entirety.

Other methods for identifying and selecting cells that have stably integrated the exogenous nucleic acid into the host cell genome can include, but are not limited to, inclusion of a reporter gene on the exogenous nucleic acid and assessment of the presence of the reporter gene in the cell, and PCR analysis and detection of the exogenous nucleic acid. In one embodiment, the cells selected, identified, or generated using the methods described herein are capable of producing higher yields of protein product than cells that are selected using only a selection method for the stable expression, e.g., integration of exogenous nucleic acid encoding the recombinant polypeptide. In an embodiment, the cells selected, identified, or generated using the methods described herein produce 2-fold, 3-fold, 4-fold, 5-fold, 6-fold, 7-fold, 8-fold, 9-fold, or 10-fold or more of the product, e.g., recombinant polypeptide, as compared to cells that were not contacted with an inhibitor of protein degradation, or cells that were only selected for stable expression, e.g., integration, of the exogenous nucleic acid encoding the recombinant polypeptide.

### Methods for Cell Line and Recombinant Polypeptide Production

The current state of the art in both mammalian and microbial selection systems is to apply selective pressure at the level of the transcription of DNA into RNA. The gene of interest is tightly linked to the selection marker making a high level of expression of the selective marker likely to result in the high expression of the gene of interest. Cells which express the selection marker at high levels are able to survive and proliferate, those which do not are less likely to survive and proliferate, e.g., apoptose and/or die. In this way a population of cells can be enriched for cells expressing the selection marker and by implication the gene of interest at high levels. This method has proved very successful for expressing straightforward proteins. In embodiments, the process described herein provides a substantially pure protein product. As used herein, “substantially pure” is meant substantially free of pyrogenic materials, substantially free of nucleic acids, and/or substantially free of endogenous cellular proteins enzymes and components from the host cell, such as polymerases, ribosomal proteins, and chaperone proteins. A substantially pure protein product contains, for example, less than 25%, 20%, 15%, 10%, 9%, 8%, 7%, 6%, 5%, 4%, 3%, 2%, or 1% of contaminating endogenous protein, nucleic acid, or other macromolecule from the host cell.

Methods for recovering and purification of a product, e.g., a recombinant polypeptide, are well established in the art. For recovering the recombinant polypeptide product, a physical or chemical or physical-chemical method is used. The physical or chemical or physical-chemical method can be a filtering method, a centrifugation method, an ultracentrifugation method, an extraction method, a lyophilization method, a precipitation method, a crystallization method, a chromatography method or a combination of two or more methods thereof. In an embodiment, the chromatography method comprises one or more of size-exclusion chromatography (or gel filtration), ion exchange chromatography, e.g., anion or cation exchange chromatography, affinity chromatography, hydrophobic interaction chromatography, and/or multimodal chromatography.

The devices, facilities and methods described herein are suitable for culturing any desired cell including prokaryotic cells and/or eukaryotic cells. The methods can be performed in, e.g., a reactor, e.g., a bioreactor. Further, in embodiments, the devices, facilities and methods are suitable for culturing suspension cells or anchorage-dependent (adherent) cells and are suitable for production operations configured for production of molecular products—such as polypeptide products—or cells and/or viruses such as those used in cellular and/or viral therapies.

In embodiments, the cells express or produce a product, such as a recombinant therapeutic or diagnostic product. As described in more detail below, examples of products produced by cells include, but are not limited to, antibody molecules (e.g., monoclonal antibodies, bispecific antibodies), fusion proteins (e.g., Fc fusion proteins, chimeric cytokines), other recombinant proteins (e.g., glycosylated proteins, enzymes, hormones), or lipid-encapsulated particles (e.g., exosomes, virus-like particles). In embodiments, the devices, facilities and methods can be used for producing biosimilars.

In embodiments, devices, facilities and methods allow for the production of eukaryotic cells, e.g., mammalian cells, and/or products of the eukaryotic cells, e.g., proteins, peptides, antibiotics or amino acids, synthesized by the eukaryotic cells in a large-scale manner. Unless stated otherwise herein, the devices, facilities, and methods can include any desired volume or production capacity including but not limited to bench-scale, pilot-scale, and full production scale capacities.

Moreover and unless stated otherwise herein, the devices, facilities, and methods can include any suitable reactor(s) including but not limited to stirred tank, airlift, fiber, microfibe, hollow fiber, ceramic matrix, fluidized bed, fixed bed, spouted bed, and/or stirred tank bioreactors. For example, in some aspects, an example bioreactor unit can perform one or more, or all, of the following: feeding of nutrients and/or carbon sources, injection of suitable gas (e.g., oxygen), flow of fermentation or cell culture medium, separation of gas and liquid phases, maintenance of temperature, maintenance of pH level, agitation (e.g., stirring), and/or cleaning/sterilizing. Example reactor units, such as a fermentation unit, may contain 1, 2, 3, 4, 5, 10, 15, 20, 25, 30, 35, 40, 45, 50, 60, 70, 80, 90, or 100, or more bioreactors. In various embodiments, the bioreactor can be suitable for batch, semi fed-batch, fed-batch, perfusion, and/or continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316 L or any other suitable stainless steel) and Inconel, plastics, and/or glass. In some embodiments, suitable reactors can be round, e.g., cylindrical. In some embodiments, suitable reactors can be square, e.g., rectangular. Square reactors may in some cases provide benefits over round reactors such as ease of use (e.g., loading and setup by skilled persons), greater mixing and homogeneity of reactor contents, and lower floor footprint.

In embodiments and unless stated otherwise herein, the devices, facilities, and methods described herein can also include any suitable unit operation and/or equipment not otherwise mentioned, such as operations and/or equipment for separation, purification, and isolation of such products. Any suitable facility and environment can be used, such as traditional stick-built facilities, modular facilities, or any other suitable construction, facility, and/or layout. For example, in some embodiments modular clean-rooms can be used. Additionally and unless otherwise stated, the devices, systems, and methods described herein can be housed and/or performed in a single location or facility or alternatively be housed and/or performed at separate or multiple locations and/or facilities.

By way of non-limiting examples and without limitation, U.S. Publication Nos. 2013/0280797; 2012/0077429; 2011/0280797; 2009/0305626; and U.S. Pat. Nos. 8,298,054; 7,629,167; and 5,656,491, which are hereby incorporated by reference in their entirety, describe example facilities, equipment, and/or systems that may be suitable.

In embodiments, the cells are eukaryotic cells, e.g., mammalian cells. The mammalian cells can be for example human or rodent or bovine cell lines or cell strains. Examples of such cells, cell lines or cell strains are e.g. mouse myeloma (NS0)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EB1, EB2, EB3, oncolytic or hybridoma-cell lines. Preferably the mammalian cells are CHO-cell lines. In one embodiment, the cell is a CHO cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells can also be avian cells, cell lines or cell strains, such as for example, EBx® cells, EB14, EB24, EB26, EB66, or EBv13.

In one embodiment, the eukaryotic cells are stem cells. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs).

In embodiments, the cultivated cells are eukaryotic cells, e.g., mammalian cells. The mammalian cells can be for example human cell lines, mouse myeloma (NSO)-cell lines, Chinese hamster ovary (CHO)-cell lines or hybridoma-cell lines. Preferably the mammalian cells are CHO-cell lines. In one embodiment, the cell is a CHO cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.).

In embodiments, the cell is a yeast cell (e.g., S. cerevisae, T. reesei), an insect cell (e.g., Sf9), an algae cell (e.g., cyanobacteria), or a plant cell (e.g., tobacco, alfalfa, Physcomitrella patens). In one embodiment, the cell is a rodent cell. In another embodiment, the cell is a HeLa, HEK293, HT1080, H9, HepG2, MCF7, Jurkat, NIH3T3, PC12, PER.C6, BHK (baby hamster kidney cell), VERO, SP2/0, NS0, YB2/0, Y0, EB66, C127, L cell, COS, e.g., COS1 and COST, QC1-3, CHO-K1.

In embodiments, the cell is a stem cell. In one embodiment, the cell is a differentiated form of any of the cells described herein. In one embodiment, the cell is a cell derived from any primary cell in culture.

In embodiments, the cell is a hepatocyte such as a human hepatocyte, animal hepatocyte, or a non-parenchymal cell. For example, the cell can be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabit hepatocytes (including New Zealand White hepatocytes). Example hepatocytes are commercially available from Triangle Research Labs, LLC, 6 Davis Drive Research Triangle Park, N.C., USA 27709.

In one embodiment, the eukaryotic cell is a lower eukaryotic cell such as e.g. a yeast cell (e.g., Pichia genus (e.g. Pichia pastoris, Pichia methanolica, Pichia kluyveri, and Pichia angusta), Komagataella genus (e.g. Komagataella pastoris, Komagataella pseudopastoris or Komagataella phaffii), Saccharomyces genus (e.g. Saccharomyces cerevisae, cerevisiae, Saccharomyces kluyveri, Saccharomyces uvarum), Kluyveromyces genus (e.g. Kluyveromyces lactis, Kluyveromyces marxianus), the Candida genus (e.g. Candida utilis, Candida cacaoi, Candida boidinii,), the Geotrichum genus (e.g. Geotrichum fermentans), Hansenula polymorpha, Yarrowia lipolytica, or Schizosaccharomyces pombe. Preferred is the species Pichia pastoris. Examples for Pichia pastoris strains are X33, GS115, KM71, KM71H; and CBS7435.

In one embodiment, the eukaryotic cell is a fungal cell (e.g. Aspergillus (such as A. niger, A. fumigatus, A. orzyae, A. nidula), Acremonium (such as A. thermophilum), Chaetomium (such as C. thermophilum), Chrysosporium (such as C. thermophile), Cordyceps (such as C. militaris), Corynascus, Ctenomyces, Fusarium (such as F. oxysporum), Glomerella (such as G. graminicola), Hypocrea (such as H. jecorina), Magnaporthe (such as M. orzyae), Myceliophthora (such as M. thermophile), Nectria (such as N. heamatococca), Neurospora (such as N. crassa), Penicillium, Sporotrichum (such as S. thermophile), Thielavia (such as T. terrestris, T. heterothallica), Trichoderma (such as T. reesei), or Verticillium (such as V. dahlia)).

In one embodiment, the eukaryotic cell is an insect cell (e.g., Sf9, Mimic™ Sf9, Sf21, High Five™ (BT1-TN-5B1-4), or BT1-Ea88 cells), an algae cell (e.g., of the genus Amphora, Bacillariophyceae, Dunaliella, Chlorella, Chlamydomonas, Cyanophyta (cyanobacteria), Nannochloropsis, Spirulina, or Ochromonas), or a plant cell (e.g., cells from monocotyledonous plants (e.g., maize, rice, wheat, or Setaria), or from a dicotyledonous plants (e.g., cassava, potato, soybean, tomato, tobacco, alfalfa, Physcomitrella patens or Arabidopsis).

In one embodiment, the cell is a bacterial or prokaryotic cell.

In embodiments, the prokaryotic cell is a Gram-positive cells such as Bacillus, Streptomyces Streptococcus, Staphylococcus or Lactobacillus. Bacillus that can be used is, e.g. the B. subtilis, B. amyloliquefaciens, B. licheniformis, B. natto, or B. megaterium. In embodiments, the cell is B. subtilis, such as B. subtilis 3NA and B. subtilis 168. Bacillus is obtainable from, e.g., the Bacillus Genetic Stock Center, Biological Sciences 556, 484 West 12^{th} Avenue, Columbus OH 43210-1214.

In one embodiment, the prokaryotic cell is a Gram-negative cell, such as Salmonella spp. or Escherichia coli, such as e.g., TG1, TG2, W3110, DH1, DHB4, DH5a, HMS 174, HMS174 (DE3), NM533, C600, HB101, JM109, MC4100, XL1-Blue and Origami, as well as those derived from E. coli B-strains, such as for example BL-21 or BL21 (DE3), all of which are commercially available.

Suitable host cells are commercially available, for example, from culture collections such as the DSMZ (Deutsche Sammlung von Mikroorganismen and Zellkulturen GmbH, Braunschweig, Germany) or the American Type Culture Collection (ATCC).

In embodiments, the cultured cells are used to produce proteins e.g., antibodies, e.g., monoclonal antibodies, and/or recombinant proteins, for therapeutic use. In embodiments, the cultured cells produce peptides, amino acids, fatty acids or other useful biochemical intermediates or metabolites. For example, in embodiments, molecules having a molecular weight of about 4000 daltons to greater than about 140,000 daltons can be produced. In embodiments, these molecules can have a range of complexity and can include posttranslational modifications including glycosylation.

The methods and process disclosed herein includes an immunogenicity calculation that is fully integrated into the development process, providing a number of advantages, including, but not limited to, (1) a method that allows immunogenicity to be performed for any production system where the genome is known, or for specific variants of the production system (e.g., GS CHO specifically as a subset of CHO), and (2) immunogenicity assessment to be performed for different patient populations (e.g. by geographic area or ethnicity). This is important as an overall average score for an HCP for the global population may mask a high score for a single particularly susceptible group.

### Numbered Embodiments

1. A simple method of rapidly analyzing a sample, e.g., to provide an assessment of the risk of a protein (e.g., the risk the protein presents if present as a contaminant in a preparation, e.g., a preparation to be administered to a subject, e.g., a pharmaceutical preparation), the method comprising:

a) providing, e.g., forming and/or maintaining, a sample mixture comprising:
i) the sample, which comprises the protein and optionally a product (e.g., a recombinant polypeptide, e.g., an antibody, enzyme, or cytokine), produced via a process; and
ii) a denaturant, e.g., deoxycholate and urea,

under conditions, e.g., concentrations of denaturant, that denatures the protein in the sample at temperature of between 10 and 30° C., e.g., 18-26° C., e.g., 20±3° C., 20±2° C., 20±1° C., or 20° C.;

b) providing, e.g., forming and or maintaining, a sample/enzyme mixture comprising:
i) sample mixture (e.g., an aliquot of sample mixture from (a)); and
ii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;

c) separating the protein digestion products using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products; and

d) assigning a protein risk score to a protein identified in the sample,

thereby analyzing the sample and providing an assessment of the risk of a protein.

2. A simple method of rapidly analyzing a sample to provide an assessment of the risk of a protein, the method comprising:

a) providing, e.g., forming and/or maintaining, a sample mixture comprising:
i) the sample, which comprises the protein (e.g., a HCP) and optionally a therapeutic product (e.g., a recombinant polypeptide), produced via a process; and
ii) a first denaturant, e.g., guanidine hydrochloride,

under conditions, e.g., concentrations of a first denaturant, that denatures the protein in the sample at temperature of between 30 and 60° C., e.g., 45-55° C., e.g., 50±3° C., 50±2° C., 50±1° C., or 50° C.;

b) providing, e.g., forming and or maintaining, a sample/enzyme mixture comprising:
i) sample mixture;
ii) a second denaturant, e.g., urea; and
iii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;

c) separating the protein digestion products using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products;

d) assigning a protein risk score to a protein identified in the sample,

thereby analyzing the sample and providing an assessment of the risk of a protein.

3. The method of either of paragraphs 1 or 2, further comprising evaluating a plurality of different samples, each made by a different process, e.g., evaluating at least 2, 4, 8, 10, 50, 96, 100, 192, 200, 500 or 1,000, different samples.

4. The method of paragraph 3, further comprising comparing the assessment of risk for a first and a second different sample.

5. The method of paragraph 4, further comprising, responsive to the comparison, selecting, a process for producing the product.

6. The method of paragraph 4, further comprising, responsive to the comparison, selecting, classifying, or further processing one of the samples.

7. A method of evaluating a process of making a product, e.g., an evaluation that incorporates assessment of the risk presented by a protein other than the product, e.g., a contaminant, produced by the process, comprising:

a) providing, e.g., forming and/or maintaining, a sample mixture comprising:
i) the protein and optionally a product (e.g., a recombinant polypeptide, e.g., an antibody, enzyme, or cytokine), produced by the process; and
ii) a denaturant, e.g., deoxycholate and urea, or guanidine hydrochloride,

under conditions, e.g., concentrations of denaturant, that denatures the protein in the sample, e.g., at temperature of between 10 and 30° C., e.g., 18-26° C., e.g., 20±3° C., 20±2° C., 20±1° C., or 20° C.;

b) providing, e.g., forming and or maintaining, a sample/enzyme mixture comprising:
i) sample mixture (e.g., an aliquot of sample mixture from a)); and
ii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;

c) separating the protein digestion products, e.g., by using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products; and

d) assigning a protein risk score to a protein identified in the sample,

thereby evaluating a process of making a product, e.g., an evaluation that incorporates assessment of the risk presented by a protein other than the product, e.g., a contaminant, produced by the process.

8. A method of evaluating a process of making a product, e.g., an evaluation that incorporates assessment of the risk presented by a protein other than the product, e.g., a contaminant, produced by the process, comprising:

a) providing, e.g., forming and/or maintaining, a sample mixture comprising:
i) the protein and optionally a product (e.g., a recombinant polypeptide, e.g., an antibody, enzyme, or cytokine), produced by the process; and
ii) a first denaturant, e.g., guanidine hydrochloride,

under conditions, e.g., concentrations of denaturant, that denatures the protein in the sample, e.g., at temperature of between 30 and 60° C., e.g., 45-55° C., e.g., 50±3° C., 50±2° C., 50±1° C., or 50° C.;

b) providing, e.g., forming and or maintaining, a sample/enzyme mixture comprising:
i) sample mixture (e.g., an aliquot of sample mixture from a));
ii) a second denaturant, e.g., urea; and
iii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;

c) separating the protein digestion products, e.g., by using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products; and

d) assigning a protein risk score to a protein identified in the sample,

thereby evaluating a process of making a product, e.g., an evaluation that incorporates assessment of the risk presented by a protein other than the product, e.g., a contaminant, produced by the process.

9. The method of either of paragraphs 7 or 8, further comprising evaluating a plurality of different processes of making a product, e.g., evaluating at least 2, 4, 8, 10, 50, 96, 100, 192, 200, 500 or 1,000, different processes.

10. The method of paragraph 9, further comprising comparing the evaluation for a first and a second different process.

11. The method of paragraph 10, further comprising, responsive to the comparison, selecting a process of making the product.

12. A method of evaluating a method of manufacturing a product, e.g., a recombinant polypeptide, e.g., an antibody, enzyme, or cytokine, to provide an assessment of risk, (e.g., the risk presented by inclusion of a protein other than the product in a preparation of the product) comprising:

a) providing, e.g., forming and/or maintaining, a sample mixture comprising:
i) one or more proteins and the product, e.g., recombinant polypeptide, produced via the method of manufacturing; and
ii) a denaturant, e.g., deoxycholate and urea,

under conditions, e.g., concentrations of denaturant, that denatures the protein in the sample at temperature of between 10 and 30° C., e.g., 18-26° C., e.g., 20±3° C., 20±2° C., 20±1° C., or 20° C.;

b) providing, e.g., forming and or maintaining, a sample/enzyme mixture comprising:
i) sample mixture (e.g., an aliquot of sample mixture from (a)); and
ii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;

c) separating the protein digestion products using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products;

d) assigning a protein risk score to a protein identified in the sample;

optionally wherein (d) is repeated for a plurality of proteins, e.g., all proteins identified by the protein digestion products; and

e) assigning a process risk score to the method of manufacturing,

thereby evaluating the method of manufacturing a product, e.g., a recombinant polypeptide, to provide an assessment of risk.

13. A method of evaluating a method of manufacturing a product, e.g., a recombinant polypeptide, e.g., an antibody, enzyme, or cytokine, to provide an assessment of risk, (e.g., the risk presented by inclusion of a protein other than the product in a preparation of the product) comprising:

a) providing, e.g., forming and/or maintaining, a sample mixture comprising:
i) one or more proteins and the product, e.g., recombinant polypeptide, produced via the method of manufacturing; and
ii) a first denaturant, e.g., guanidine hydrochloride,

under conditions, e.g., concentrations of denaturant, that denatures the protein in the sample at temperature of between 30 and 60° C., e.g., 45-55° C., e.g., 50±3° C., 50±2° C., 50±1° C., or 50° C.;

b) providing, e.g., forming and or maintaining, a sample/enzyme mixture comprising:
i) sample mixture (e.g., an aliquot of sample mixture from (a));
ii) a second denaturant, e.g., urea, and
iii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;

c) separating the protein digestion products using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products;

d) assigning a protein risk score to a protein identified in the sample;

optionally wherein (d) is repeated for a plurality of proteins, e.g., all proteins identified by the protein digestion products; and

e) assigning a process risk score to the method of manufacturing,

thereby evaluating the method of manufacturing a product, e.g., a recombinant polypeptide, to provide an assessment of risk.

14. The method of either of paragraphs 12 or 13, further comprising evaluating a plurality of different methods of manufacturing a product, e.g., evaluating at least 2, 4, 8, 10, 50, 96, 100, 192, 200, 500 or 1,000, different methods of manufacturing a product.

15. The method of paragraph 14, further comprising comparing the assessment of risk for a first and a second different method of manufacturing a product.

16. The method of paragraph 14, further comprising, responsive to the comparison, selecting or classifying, a method of manufacturing the product.

17. The method of any of paragraphs 1-16, wherein the protein is a contaminant or other undesirable component (e.g., a fragment, denatured, or mis-folded version of a product being produced by a set of conditions, or a host cell protein (HCP) or fragment thereof).

18. The method of any of paragraphs 1-17, wherein the denaturant, first denaturant, or second denaturant comprises, consists of, or consists essentially of deoxycholate and urea, guanidine hydrochloride, or urea and guanidine hydrochloride.

19. The method of any of paragraphs 1-18, wherein the denaturant or first denaturant comprises, consists of, or consists essentially of guanidine hydrochloride.

20. The method of any of paragraphs 1-18, wherein the denaturant or first denaturant comprises, consists of, or consists essentially of urea and deoxycholate.

21. The method of any of paragraphs 1-18, wherein the denaturant or second denaturant comprises, consists of, or consists essentially of urea.

22. The method of any of paragraphs 1-21, wherein the concentration of denaturant in the sample mixture is higher than the concentration of denaturant in the sample/enzyme mixture.

23. The method of any of paragraphs 1-22, wherein:

the concentration of denaturant in sample mixture is sufficiently high to denature the protein, e.g., wherein at least 50, 60, 70, 80, 90, 95, 96, 97, 98, 99, or 100% of the protein is denatured; and

the concentration of denaturant in sample/enzyme mixture is sufficiently low to not denature the enzyme, e.g., wherein less than 50, 40, 30, 20, 10, 5, 4, 3, 2, or 1% of the enzyme is denatured.

24. The method of any of paragraphs 1-23, wherein combination of the enzyme preparation with the sample mixture dilutes (i.e., reduces the concentration of) the denaturant such that it does not denature the enzyme.

25. The method of any of paragraphs 1-24, wherein the concentration of the denaturant in the sample mixture is:

i) at least 1, 1.5, 2, 2.5, 3, 3.5, 4, 4.5, 5, 5.5, 6, 6.5, 6.6, 6.7, 6.8, 6.9, 7, 7.5, or 8;

ii) 1-10 M, 2-9 M, 3-8 M, 4-7 M, 5-7 M, 6-6.6 M, 6 M, 6.6 M, or 8 M;

iii) 0-10 M, 2-9 M, 3-8 M, 4-7 M, 5-7 M, 0.5-5 M, 0.5-2 M, 0.5 M, 1 M, or 2 M; or

iv) 0.01%-50%, 1%-40%, 1%-20%, 0.5%-10%, 0.01%-5%, or 0.1%-2% (m/v).

26. The method of any of paragraphs 1-25, wherein the concentration of the denaturant in the sample/enzyme mixture is:

i) less than or equal to 4, 3.5, 3, 2.5, 2, 1.5, 1, 0.5, or 0.1 M;

ii) less than or equal to 0.5 or 0.1 M, e.g., essentially 0 M;

iii) less than or equal to 2 M or 0.5 M; or

iv) less than or equal to 2%, 1%, 0.1%, 0.05%, 0.01%, 0.005%, 0.0025%, 0.001%, or 0.0001%, e.g., essentially 0% (m/v).

27. The method of any of paragraphs 1-26, wherein the sample mixture comprises a first denaturant (i.e. the denaturant of (a)(ii)), and the sample/enzyme mixture comprises the first denaturant and a second denaturant.

28. The method of paragraph 27, wherein the first denaturant is guanidine hydrochloride and the second denaturant is urea.

29. The method of either of paragraphs 27 or 28, wherein the concentration of the first denaturant in the sample mixture is 1-10 M, 2-9 M, 3-8 M, 4-7 M, 5-7 M, 6-6.6 M, 6 M, 6.6 M, or 8 M.

30. The method of any of paragraphs 27-29, wherein the concentration of the first denaturant in the sample/enzyme mixture is:

i) 0-10 M, 2-9 M, 3-8 M, 4-7 M, 5-7 M, 0.5-5 M, 0.5-2 M, 0.5 M, 1 M, or 2 M;

ii) less than or equal to 4, 3.5, 3, 2.5, 2, 1.5, 1, 0.5, or 0.1 M; or

iii) ii) less than or equal to 0.5 or 0.1 M, e.g., essentially 0 M.

31. The method of any of paragraphs 27-30, wherein the concentration of the second denaturant in the sample/enzyme mixture is:

i) less than or equal to 4, 3.5, 3, 2.5, 2, 1.5, 1, 0.5, or 0.1 M;

ii) less than or equal to 0.5 or 0.1 M, e.g., essentially 0 M; or

iii) less than or equal to 2 M or 0.5 M.

32. The method of any of paragraphs 1-31, wherein:

the pH of the sample mixture is sufficiently low that deamidation reactions are substantially inhibited e.g., wherein at least 50, 60, 70, 80, 90, 95, 96, 97, 98, 99, or 100% of the asparagine and glutamine side chains of the protein are unaltered, and

the pH of the sample/enzyme mixture is sufficiently high that the enzyme is active, e.g., wherein the enzyme is at least 50, 60, 70, 80, 90, or 100% active, e.g., operating at 50, 60, 70, 80, 90, or 100% efficiency compared to maximum efficiency.

33. The method of any of paragraphs 1-32, wherein the pH of the sample mixture is lower, e.g., at least 1, 1.5, or 2 units lower, than the pH of the sample/enzyme mixture.

34. The method of any of paragraphs 1-33, wherein the pH of the sample mixture is 5.5±1, 0.75, 0.5, or 0.25 (e.g., 5.5±0.5) and the pH of the sample/enzyme mixture is 7.3±1, 0.75, 0.5, or 0.25 (e.g., 7.3±0.5).

35. The method of any of paragraphs 1-34, wherein the pH of the sample mixture is 5.5 and the pH of the sample/enzyme mixture is 7.3.

36. The method of any of paragraphs 1-35, wherein the method does not comprise alkylation of cysteine residues of the protein or protein digestion products.

37. The method of any of paragraphs 1-36, wherein the method does not comprise exposing one or more (e.g., all) of the sample, sample mixture, or sample/enzyme mixture to conditions, e.g., the addition of a reagent, e.g., an alkylating agent, e.g., iodoacetamide, to alkylate cysteine residues of the protein or protein digestion products.

38. The method of any of paragraphs 1-37, wherein the sample mixture and/or sample/enzyme mixture comprise a reducing agent, e.g., Tris(2-carboxyethyl)phosphine (TCEP), dithiothreitol (DTT), or beta-mercaptoethanol.

39. The method of paragraph 38, wherein the concentration of reducing agent, e.g., Tris(2-carboxyethyl)phosphine (TCEP), dithiothreitol (DTT), or beta-mercaptoethanol, in the sample mixture is higher than in sample/enzyme mixture.

40. The method of either of paragraphs 38 or 39, wherein:

the concentration of reducing agent, e.g., Tris(2-carboxyethyl)phosphine (TCEP), dithiothreitol (DTT), or beta-mercaptoethanol, in the sample mixture is sufficiently high to substantially reduce the cysteines of the protein, e.g., wherein at least 50, 60, 70, 80, 90, 95, 96, 97, 98, 99, or 100% of the cysteine residues of the protein are reduced; and the concentration of reducing agent, e.g., Tris(2-carboxyethyl)phosphine (TCEP), dithiothreitol (DTT), or beta-mercaptoethanol, in the sample/enzyme mixture is sufficiently low to not interfere with other steps of the method or method of manufacturing, e.g., wherein the reducing agent does not significantly accumulate in equipment (e.g., mass spectrometer or analytical column) or produce additional signal in data (e.g., mass spectrometry data).

41. The method of any of paragraphs 38-40, wherein the concentration of the reducing agent in the sample mixture is at least 1, 2, 3, 4, 5, 6, 7, 8, 9, 10, 11, 12, 13, 14, 15, 16, 17, 18, 19, or 20 mM, e.g., 1, 2, 3, 4, 5, 6, 7, 8, 9, 10, 11, 12, 13, 14, 15, 16, 17, 18, 19, or 20 mM.

42. The method of any of paragraphs 38-41, wherein the concentration of the reducing agent in the sample/enzyme mixture is less than or equal to 10, 9, 8, 7, 6, 5, 4, 3, 2, 1, 0.5, or 0.1 mM, e.g., 10, 9, 8, 7, 6, 5, 4, 3, 2, 1, 0.5, or 0.1 mM.

43. The method of any of paragraphs 38-42, wherein the reducing agent is Tris(2-carboxyethyl)phosphine (TCEP).

44. The method of any of paragraphs 1-43, wherein the protein digestion products are separated on the basis of one or more (e.g., one, two, three or more) of size, charge, or affinity.

45. The method of any of paragraphs 1-44, wherein separating the protein digestion products comprises using chromatography, e.g., 1-dimensional chromatography, e.g., affinity chromatography, gel filtration chromatography, ion exchange chromatography, reversed phase chromatography, hydrophobic interaction chromatography, high performance liquid chromatography (HPLC), gas chromatography (GC), capillary electrophoresis, ion mobility, or any chromatographic method described herein.

46. The method of any of paragraphs 1-45, wherein (c) further comprises providing the identity of a protein digestion product, e.g., by mass spectroscopy, e.g., LC/MS, tandem mass spectrometry, or RP-LCMS².

47. The method of any of paragraphs 1-46, wherein (c) comprises separating the protein digestion products using chromatography, e.g., 1-dimensional chromatography, and providing the identity of the protein digestion products, e.g., by mass spectrometry, e.g., LC/MS, tandem mass spectrometry, or 1D RP-LCMS².

48. The method of any of paragraphs 1-47, wherein providing the identity of a protein digestion product, e.g., by mass spectrometry, comprises evaluating the mass, charge, retention or elution time, and optionally intensity (e.g., abundance or amount) for one or a plurality of protein digestion products, or of each protein digestion product.

49. The method of any of paragraphs 1-48 comprising, for one or a plurality of protein digestion products, or each of a plurality of protein digestion products, assigning a value (e.g., a numerical value or a value related to position on a display of a plurality of protein digestion products) that is a function of one or more or all of mass, charge, retention or elution time, and optionally intensity (e.g., abundance or amount), of a protein digestion product.

50. The method of paragraph 49, comprising, displaying the values on a 3 dimensional representation, e.g., wherein an axis, e.g., the y axis represents a value that is a function of the ratio of mass to charge, a second axis, e.g., the x axis represents a value that is a function of retention or elution time, and a third axis, e.g., the z axis, represents intensity (e.g., abundance or amount) of the protein digestion product.

51. The method of either of paragraphs 49 or 50, wherein the value is a function of mass, charge, retention time, and intensity (e.g., abundance or amount).

52. The method of any of paragraphs 49-51, comprising comparing the value of a protein digestion product from a first sample with the value of the protein digestion product from a second sample.

53. The method of paragraph 52, wherein the amount of the protein digestion product in the first sample is greater, e.g., at least 10, 20, 30, 40, 50, 60, 70, 80, 90, 100, 200, 500, or 1000% greater than the amount of the protein digestion product from the second sample.

54. The method of paragraph 52, wherein the amount of the protein digestion product in the second sample is greater, e.g., at least 10, 20, 30, 40, 50, 60, 70, 80, 90, 100, 200, 500, or 1000% greater than the amount of the protein digestion product from the first sample.

55. The method of any of paragraphs 49-54, comprising providing a correlation between value and identity for one or a plurality of protein digestions products.

56. The method of any of paragraphs 1-55, further comprising identifying or classifying a protein digestion product in a test sample comprising:

providing a value, e.g., a value that is a function of mass, charge, retention or elution time, and optionally intensity (e.g., abundance or amount) of the protein digestion product from the test sample;

responsive to that value, identifying or classifying the protein digestion product from the test sample.

57. The method of paragraph 56, comprising comparing the value for the protein digestion product from the test sample with a reference value, e.g., the value for a protein digestion product of known identity, structure or composition.

58. The method of paragraph 57, wherein responsive to the comparison of the value for the protein digestion product from the first sample and the reference value, classifying or assigning an identity, structure, or composition to the protein digestion product from the first sample.

59. The method of any of paragraphs 1-58, wherein a plurality, e.g., at least 10, 20, 30, 40, 50, 60, 70, 80, 90, or 100% of protein digestion products in a sample are classified or assigned an identity, structure or composition.

60. The method of any of paragraphs 49-59, wherein providing the identity of a protein digestion product, e.g., by mass spectrometry, further comprises comparing the value for the protein digestion product from at least 2, 10, 20, 96, 100, 192, 1,000, or 10,000 samples with a reference value, e.g., the value for a protein digestion product of known identity, structure or composition.

61. The method of any of paragraphs 49-60, wherein providing the identity of a protein digestion product, e.g., by mass spectrometry, further comprises, responsive to the similarity of the value for the protein digestion product from the samples and a reference value, classifying or assigning an identity, structure or composition to the protein digestion product from the at least 2, 10, 20, 96, 100, 192, 1,000, or 10,000 samples.

62. The method of any of paragraphs 1-61, wherein a plurality, e.g., at least 10, 20, 30, 40, 50, 60, 70, 80, 90, or 100% of protein digestion products in at least 2, 10, 20, 96, 100, 192, 1,000, or 10,000 samples are classified or assigned an identity, structure or composition.

63. The method of any of paragraphs 1-62, wherein the method is performed for a plurality of samples, and one or more of the plurality of samples is produced by a different process or method of manufacturing, e.g., allowing a condition or set of conditions (e.g., one or more of: denaturant selection, denaturant concentration, pH, temperature, reducing agent, incubation time, and other conditions described herein) to be identified that optimize, e.g., minimize or maximize, the levels of one or more protein digestion products.

64. The method of any of paragraphs 1-63, wherein the method is repeated to analyze a plurality of samples, wherein each sample is analyzed under a plurality of conditions, e.g., at least 2, 10, 50, 96, 100, 192, 1,000, or 10,000 conditions, and wherein the method further comprises, responsive to the analysis of the plurality of samples, selecting a sample from each of said plurality of conditions.

65. The method of any of paragraphs 1-64, wherein the method further comprises classifying or selecting a process or method of manufacturing, e.g., a process or method of manufacturing that results in a preselected or optimized level of one or more protein digest products.

66. The method of any of paragraphs 1-65, wherein the method further comprises classifying or selecting a process or method of manufacturing, e.g., a process or method of manufacturing that results in a preselected or optimized level of one or more proteins.

67. The method of any of paragraphs 1-66, wherein the protein risk score is a function of one or more of:

an unwanted, e.g., off-target, property in a subject to receiving a preparation comprising the protein and, optionally, a product, e.g., immunogenicity;

an unwanted effect of the protein in a preparation of the product, e.g., a preparation of a drug, e.g., the propensity to cause denaturation, precipitation, or color; and

a value for the abundance of the protein present in the sample.

68. The method of any of paragraphs 1-67, wherein step (d) is repeated to provide a protein risk score for one or more (e.g., at least 2, 10, 50, 100, 200, 500, 1000, or all) proteins identified in the sample.

69. The method of any of paragraphs 1-68, wherein step (d) comprises providing a protein risk score, e.g., an immunogenicity risk score, e.g., as generated by the Epibase® platform.

70. The method of any of paragraphs 1-69, wherein step (d) comprises providing a immunogenicity risk score as generated by the Epibase® platform.

71. The method of any of paragraphs 1-70, further comprising providing a process risk score to the sample.

72. The method of paragraph 71, wherein the process risk score is a function of the one or more protein risk scores of the sample's proteins.

73. The method of either of paragraphs 71 or 72, wherein the process risk score is calculated based upon the formula:

Process Risk Score=Σ([Protein Abundance]×[Immunogenicity Risk Score]).

74. The method of any of paragraphs 71-73, wherein the method is repeated to analyze a plurality of samples, e.g., at least 2, 10, 50, 96, 10, 192, or 1000, and

wherein two or more (e.g., all) of the samples are provided using a different process or method of manufacturing, and wherein a process risk score is provided for a plurality of samples (e.g., all samples), or

wherein two or more (e.g., all) of the samples are provided at different time points during a process or method of manufacturing, and wherein a process risk score is provided for a plurality of samples (e.g., all samples).

75. The method of any of paragraphs 71-74, comprising comparing the process risk score of a process or method of manufacturing, e.g., the process or method of manufacturing used to provide the sample, with a reference.

76. The method of any of paragraphs 71-75, comprising comparing the process risk score of a first process or method of manufacturing with a process risk score for a second process or method of manufacturing.

77. The method of any of paragraphs 71-75, comprising comparing the process risk score of a process at a first time point with the process risk score of a process at a second time point.

78. The method of paragraph 76, comprising, responsive to the comparison, selecting one of the processes or methods of manufacturing, e.g., for further analysis or for further use, e.g., to make the product, e.g., recombinant polypeptide.

79. The method of either of paragraphs 75 or 77, comprising, responsive to the comparison, altering a parameter of a process or method of manufacturing, e.g., a level of or presence of: a media supplement, oxygen, a multivaltent cation, ammonium, iron, nitrogen, phosphate, calcium, magnesium, manganese, a flocculating or clarifying agent (e.g., alum, aluminium, chlorohydrate, aluminium sulphate, calcium oxide, calcium hydroxide, iron(II) sulphate (ferrous sulphate), iron(III) chloride (ferric chloride), polyacrylamide, polyDADMAC, sodium aluminate, sodium silicate), a selection reagent (e.g., an antibiotic, e.g., neomycin, blasticidin, hygromyocin, puromycin, zeocin, mycophenolic acid), sodium butyrate, or an amino acid.

80. The method of either of paragraphs 75 or 77, comprising, responsive to the comparison, continuing a process or method of manufacturing without altering a parameter of the process or method of manufacturing, e.g., a level of or presence of: a media supplement, oxygen, a multivaltent cation, ammonium, iron, nitrogen, phosphate, calcium, magnesium, manganese, a flocculating or clarifying agent (e.g., alum, aluminium, chlorohydrate, aluminium sulphate, calcium oxide, calcium hydroxide, iron(II) sulphate (ferrous sulphate), iron(III) chloride (ferric chloride), polyacrylamide, polyDADMAC, sodium aluminate, sodium silicate), a selection reagent (e.g., an antibiotic, e.g., neomycin, blasticidin, hygromyocin, puromycin, zeocin, mycophenolic acid), sodium butyrate, or an amino acid.

81. A method of manufacturing a product, e.g., a recombinant polypeptide, comprising providing a sample comprising the product, wherein the sample is analyzed by a method of analyzing a sample of any of paragraphs 1-6, and 17-80.

82. The method of any of paragraphs 1-81, wherein the process or method of manufacturing comprises expression and secretion from a plurality of cells (e.g., a plurality of CHO cell, e.g., a plurality of GS-CHO cells).

83. A method of detecting, monitoring, identifying, or quantifying a host cell protein (HCP) in a recombinant polypeptide sample, the method comprising:

a) generating or obtaining a sample comprising a recombinant polypeptide expressed and secreted from a culture of cells (e.g., a CHO, e.g., a GS-CHO, cell culture);

b) separating a plurality of HCP components (e.g., fractionated or cleaved HCP components) using chromatagrophy, e.g., 1-dimensional chromatography, and determining an identifying characteristic of each HCP component of the plurality of separated HCP components;

c) evaluating the identifying characteristic of each HCP of the plurality;

thereby detecting, monitoring, identifying, or quantifying a host cell protein (HCP) in a recombinant polypeptide sample; and

optionally d) repeating (a)-(c) for a plurality of samples, e.g., samples provided at different times from a process or method of manufacturing, and evaluating an identifying characteristic, e.g., the risk of a protein or proteins, across the plurality of samples.

84. A method of evaluating a method of manufacturing a product, e.g., a recombinant polypeptide, the method comprising:

a) generating or obtaining a sample comprising a recombinant polypeptide expressed and secreted from a culture of cells (e.g., a CHO, e.g., a GS-CHO, cell culture);

b) separating a plurality of HCP components (e.g., fractionated or cleaved HCP components) using chromatography, e.g., 1-dimensional chromatography, and determining an identifying characteristic of each HCP component of the plurality of separated HCP components;

c) evaluating the identifying characteristic of each HCP of the plurality; and

optionally d) repeating (a)-(c) for a plurality of samples, e.g., samples provided by different methods of manufacturing a product, and evaluating an identifying characteristic, e.g., the risk of a protein or proteins, across the plurality of methods of manufacturing,

thereby evaluating the method of manufacturing.

85. The method of any of paragraphs 1-84, wherein the sample comprises culture supernatant.

86. The method of any of paragraphs 1-84, wherein the sample comprises cell lysate.

87. The method of any of paragraphs 1-86, wherein the sample comprises culture supernatant and cell lysate.

88. The method of any of paragraphs 1-87, wherein the product or recombinant polypeptide is a homopolymeric or heteropolymeric polypeptide, e.g., a hormone, growth factor, receptor, antibody, cytokine, receptor ligand, transcription factor or enzyme, preferably an antibody or an antibody fragment, e.g., a human antibody or a humanized antibody or fragment thereof, e.g., a humanized antibody or fragment thereof derived from a mouse, rat, rabbit, goat, sheep, or cow antibody, typically of rabbit origin.

89. The method of any of paragraphs 1-88, wherein the product or recombinant polypeptide is a therapeutic polypeptide.

90. The method of any of paragraphs 1-89, wherein the product or recombinant polypeptide is one disclosed in Table 1, Table 2, Table 3, or Table 4.

91. The method of any of paragraphs 1-90, wherein the product or recombinant polypeptide is an antibody.

92. The method of paragraph 91, wherein the antibody is a monoclonal antibody.

93. The method of paragraph 92, wherein the monoclonal antibody is a therapeutic antibody.

94. The method of any of paragraphs 82-93, wherein the cells are mammalian cells.

95. The method of paragraph 94, wherein the host cell is a mouse, rat, Chinese hamster, Syrian hamster, monkey, ape, dog, horse, ferret, or cat.

96. The method of paragraph 95, wherein the cells are Chinese hamster ovary (CHO) cells.

97. The method of paragraph 96, wherein the CHO cells are CHO-K1 cells, CHO-K1 SV cells, DG44 CHO cells, DUXB11 CHO cells, CHOS cells, CHO GS knock-out cells, CHO FUT8 GS knock-out cells, CHOZN cells, or CHO-derived cells.

98. The method of any of paragraphs 82-97, wherein the cells are Hela, HEK293, HT1080, H9, HepG2, MCF7, Jurkat, NIH3T3, PC12, PER.C6, BHK (baby hamster kidney cell), VERO, SP2/0, NS0, YB2/0, Y0, EB66, C127, L cell, COS, e.g., COS1 and COST, QC1-3, or any cells derived therefrom.

paragraph

99. The method of any of paragraphs 1-98, wherein the method takes no longer than 120, 96, 72, 48, 24, 12, 6, 4, or 3 hours.

100. A database (e.g., memorialized or recorded on a computer readable medium) comprising a library of identifying characteristics for HCPs or protein digestion products and protein risk scores derived from cell culture supernatant of a cell culture (e.g., a CHO, eg., a GS-CHO, cell culture).

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### Example 1: Methods

The following describe methods used and referenced throughout Examples 2, 3, and 4.

Test Samples

cB72.3 IgG4k antibody was produced from a 1000 L scale fermentation bioreactor culture. Material was either harvested using a standard primary recovery filtration train or treated by addition of 0.1% (final) polydiallyldimethylammonium chloride and harvested using a Millipore Clarisolve filter. For each batch of material, samples were provided of (1) clarified cell culture supernatant, (2) neutralised eluate from Protein A (MAbSelect SuRe) chromatography, (3) eluate from anion exchange (Sartobind Q) chromatography.

HCP ELISA

An immobilised anti-CHO HCP polyclonal antibody (raised against proteins derived from a null-transfected CHO cell line) was used to capture residual HCP in the test samples. The bound HCP was detected using the same polyclonal antibody conjugated to biotin, which was in turn detected by horseradish peroxidase-conjugated extravadin. 3,3′,5,5′ tetramethylbenzidine (TMB) was used as the chromogenic substrate).

Sample Preparation

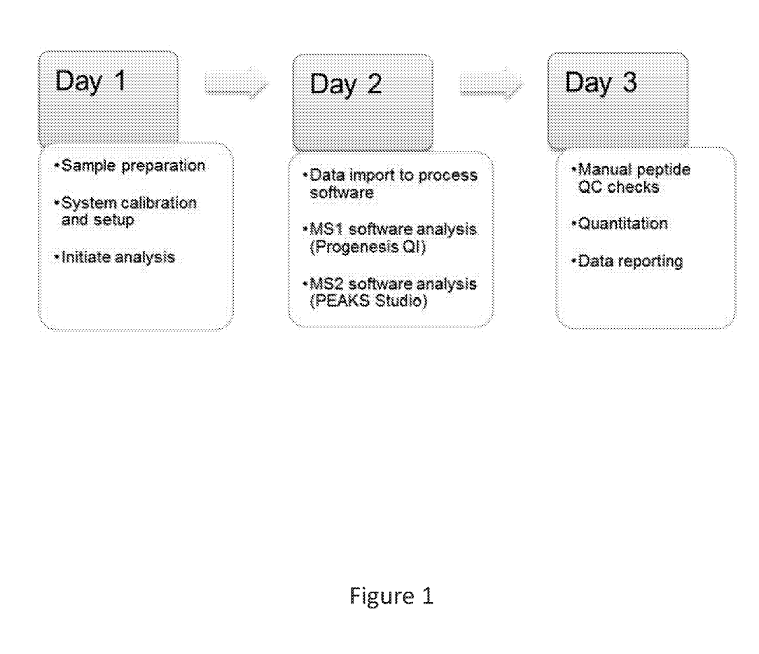

A general tryptic digestion protocol for the structural analysis of protein therapeutics was used for sample preparation. Protein concentration in each sample was determined by absorbance measurements at 280 nm and by reference to the theoretically calculated extinction coefficient for cB72.3. Three technical replicates from separate sample preparations were analysed for each sample. All raw data was processed in parallel using PEAKS Studio (for peptide and protein identification) and Progenesis QI for Proteomics (for label free quantitation, including normalisation across samples). Peptide identifications were imported into Progenesis QI for data collation. Hi3 quantitation was performed manually based on the summed, normalised intensities of all detected charge states for the 3 highest-intensity peptides for each protein. Proteins were only subject to quantitation where at least 3 peptides were detected. A top level overview of the proposed analysis process is shown in FIG. 1.

Sample Preparation for LC-MS/MS

Samples were denatured in 6.6M guanidine HCl, reduced with TCEP and digested with trypsin prior to analysis. Three replicates were prepared for each sample. It is anticipated that any sample preparation method that can effectively produce peptides from a protein mixture into a buffer matrix compatible with mass spectrometry could be used with this analytical approach.

LC-MS/MS Data Acquisition

Data were acquired using a Dionex RSSLnano nanoLC system coupled to a Thermo Fusion Tribrid Q-OT-qIT mass spectrometer. A 1 μl volume of the tryptic peptides for each sample was injected onto a PepMap100 C18 300 Å Nano-Trap column (Thermo) in a loading buffer of 98:2 water:acetonitrile plus 0.08% TFA at 12 μl/min for 3 minutes. After 3 minutes the nanoLC flow was directed in the reverse direction through the trapping column onto the analytical column (EasySpray RSLC C18 2 μm, 100 Å, 75 μm×25 cm (Thermo)). A linear gradient was applied between 0.1% formic acid in water and 0.1% formic acid in 80:20 acetonitrile:water.

Source ionisation settings were static during the acquisition at 2500 V spray voltage and a transfer tube temperature of 275° C. The mass spectrometer was configured in positive ionisation mode for acquisition of MS1 data in the orbitrap at 120,000 FWHM (full width at half maximum) nominal resolution with quadrupole isolation over a range of 350-1,550 m/z, an AGC target of 2.0e5 and a maximum injection time of 50 ms. Data was mass-corrected using an internal standard based on a flouranthine ion lockmass at generated from a separate reagent ion source. Only charge states between z=2 and z=6 were selected for MS2 fragmentation.

MS² fragmentation was performed in the linear ion trap at normalised collision energy of 28%, an AGC target of 1.0e4 and a maximum scan time of 200 ms at the “Normal” trap scan rate.

Mass Spectrometry Data Analysis

Protein identifications based on MS² fragmentation were performed using PEAKS Studio software. Protein identification was performed for cell culture supernatant (CCCS) only. False discovery rate at the peptide level was controlled at <0.1% using decoy fusion methodology (Zhang, J, et al., “PEAKS DB: de novo sequencing assisted database search for sensitive and accurate peptide identification”, Mol. Cell Proteomics 4(11), 111 (2012)). At least 3 unique peptides were required for each protein assignment. Mass tolerances were specified at <7.5 ppm for parent ions and <0.3 Da for fragment ions. Identification were made against Rodentia taxa within the TrEMBL database.

MS¹ data processing for all samples was performed using Progenesis QI for Proteomics software. Sample data was imported, automatically retention time aligned and responses normalised against a control supernatant sample. Peak detection was performed at “highest” sensitivity setting. Peptide identifications were imported and assignments were visually examined to ensure quantitation was performed exclusively against peptides that did not show spectral interference. Relative quantitation was performed based on Hi3 methodology.

In Silico Immunogenicity Analysis

Sequences for each identified protein were submitted for in silico immunogenicity assessment using the Epibase® platform. Epibase® is an in silico platform for immunogenicity risk screening (Walle Van, I, et al., “Immunogenicity screening in protein drug development,” Expert Opin Biol Ther 7(3), 405 (2007)). The platform identifies potential T cell epitopes in a protein sequence by predicting the binding affinities of all 10-mer peptides derived from the sequence to HLA class II receptors.

The screening was performed targeting the Global population. The Global population HLA set includes 85 HLA class II allotypes, in particular 43 DRB1 allotypes which are a primary focus of immunogenicity profiling. A human proteome filter, including the top 25% most abundant human proteins, was used to filter-out self-peptides (peptides which are presented on HLA molecules but will not bind T cell receptors). The immunogenicity risk score for a protein is obtained by taking into account the number of predicted T cell epitopes in a protein and population frequencies of affected HLA allotypes.

### Example 2: Creation of a CHO HCP Library

The methods provided in this example describe the generation of a CHO HCP library used in the methods described in Example 2.

A reference library of MS¹ peaks derived from HCPs determined from CCCS of CHO cell culture was generated. The reference library defined windows in retention time and m/z space that was then applied to data acquired for purified samples. Since the CCCS derived from the production bioreactor in a mammalian cell expression system contains every CHO protein that could be present as an HCP in the integration regions, this can subsequently be applied to all purified samples within the same analysis. Identification and quantification aspects of HCP analysis are therefore decoupled, although a sample of each relevant specific CCCS for every purified sample in the analysis is required, but is not generally a limitation for bioprocess development. Use of a CCCS-derived integration library avoids the necessity for multiple analysis runs.

A total of 627 protein identifications were obtained across all of the CCCS samples analyzed. Of this population, 259 proteins were of sufficient abundance to allow peak detection and quantification in the MS¹ processing software. The sensitivity difference between the limit of detection and limit of quantitation can be attributed to the different mechanisms that gave rise to each value. Limit of detection is defined from MS² data by the p values for peptide sequence matches generated from ion trap fragmentation data. Relatively high ion injection times into the trap used in this work resulted in high MS² detection sensitivity. Additionally, MS² detection was possible based on only a single MS¹ parent ion scan. By contrast the peak detection algorithm used for quantitation of MS¹ data required that a well-defined chromatographic peak is present across multiple scans—signals that appear in a small number of scans will typically not pass data filtering QC checks. The requirement for chromatographic peak detection also drove use of a relatively rapid MS¹ scan time at 120,000 FWHM resolution (rather than a lower scan rate at up to 500,000 FWHM resolution).

Relative quantitation was performed based on Hi3 approaches (J. C. Silva, et al., “Absolute quantification of proteins by LCMSE: a virtue of parallel MS acquisition,” Mol. Cell Proteomics. 5(1), 144 (2006)). Using mAb product peptide peaks as quantitation standards, MS¹ peak detection in supernatant samples was possible at down to a reported threshold of 10 ppm. Total HCP results obtained by LC-MS were compared to results obtained for the same samples tested using a standard CHO HCP ELISA. Briefly, an immobilised anti-CHO HCP polyclonal antibody (raised against proteins derived from a null-transfected CHO cell line) was used to capture residual HCP in the test samples. The bound HCP was detected using the same polyclonal antibody conjugated to biotin, which was in turn detected by horseradish peroxidase-conjugated extravadin. 3,3′,5,5′ tetramethylbenzidine (TMB) was used as the chromogenic substrate). HCP was quantified against a standard curve generated within the assay. Interassay controls were included in all assays. Spike recovery of HCP met the acceptance criterion of 100%±25%. Results are shown in Table 5. MSS refers to MabSelect SuRe.

TABLE 5
Comparison of Total HCP Quantitation by ELISA and NanoLC-MS/MS

| | HCP ng/mg protein | | | | | |
|---|---|---|---|---|---|---|
| | | MSS | SBQ | CCCS + | MSS Eluate + | SBQ Eluate + |
| Test Method | CCCS | Eluate | Eluate | pDADMAC | pDADMAC | pDADMAC |
| HCP | NT | 167 | 79 | NT | 44 | 29 |
| ELISA | | | | | | |
| (UKSL-90) | | | | | | |
| NanoLC-MS² | 139,738 | 2,378 | 2,062 | 119,171 | 2,376 | 2,350 |

NT Not Tested

Reported total HCP values determined by mass spectrometry were between 10- to 100 fold higher than reported by ELISA, however were broadly consistent with values obtained in previous work by other groups of between 611 ppm and 10,000 ppm in purified and partially purified mAb products (C. E. Doneanu, et al., “Analysis of host-cell proteins in biotherapeutic proteins by comprehensive online two-dimensional liquid chromatography/mass spectrometry,” MAbs. 4(1), 24 (2012); A. Farrell, et al., “Quantitative Host Cell Protein Analysis Using Two Dimensional Data Independent LC-MS(E),” Anal. Chem. 87(18), 9186 (2015); M. R. Schenauer, G. C. Flynn, and A. M. Goetze, “Identification and quantification of host cell protein impurities in biotherapeutics using mass spectrometry,” Anal. Biochem. 428(2), 150 (2012); Q. Zhang, et al., “Comprehensive tracking of host cell proteins during monoclonal antibody purifications using mass spectrometry,” MAbs. 6(3), 659 (2014)).

### Example 3: Effect of (Polydiallyldimethylammonium Chloride) on HCP Challenge to DSP

The methods provided in this example are related to the use of proteomic based analysis of HCPs using 1D chromatography as a tool for, inter alia, monitoring and developing a process for the production of recombinant therapeutic polypeptides, e.g., antibodies. This example, describes a live process development study, in which the impact of a new process chemical, namely the flocculent polydiallyldimethylammonium chloride, used during primary recovery of a production culture was determined in terms of HCP clearance for the purification process.

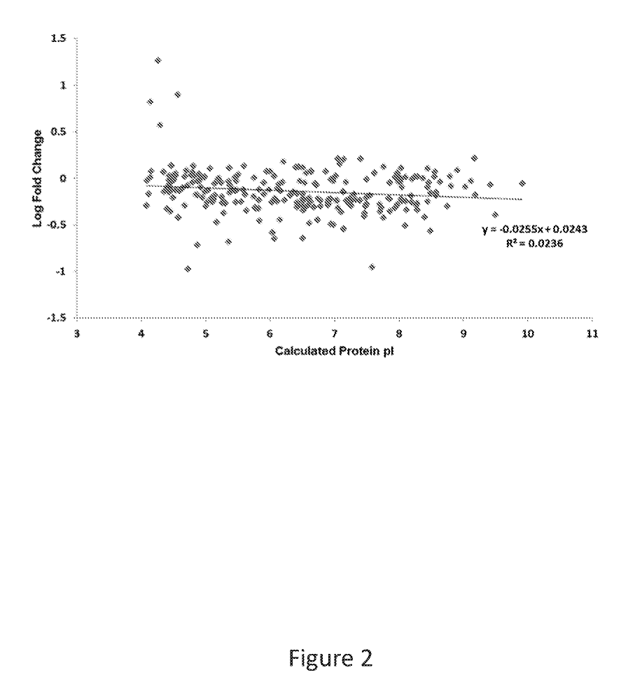

HCPs between equivalent samples from streams with and without pDADMAC were compared in terms of abundance. False discovery rate was controlled at 5%. Out of the 259 proteins for which quantitation data was obtained, 121 proteins showed a significant (p<0.05, q<0.05) decrease in CCCS samples that had been treated with pDADMAC compared to CCCS without pDADMAC treatment. pI values for each protein quantified in the experiment were calculated to determine if pDADMAC-mediated HCP removal was correlated with protein pI, and no overall correlation was observed (FIG. 2), but it was noted that the proteins showing a greater than 2-fold increase in abundance in the presence of pDADMAC all had calculated pI values of <5. pI calculations were performed based on amino acid sequence only: solvent accessible charge information was not included in this analysis and it is likely that charge based interactions are mediated by specific patches on protein surfaces rather than overall charge.

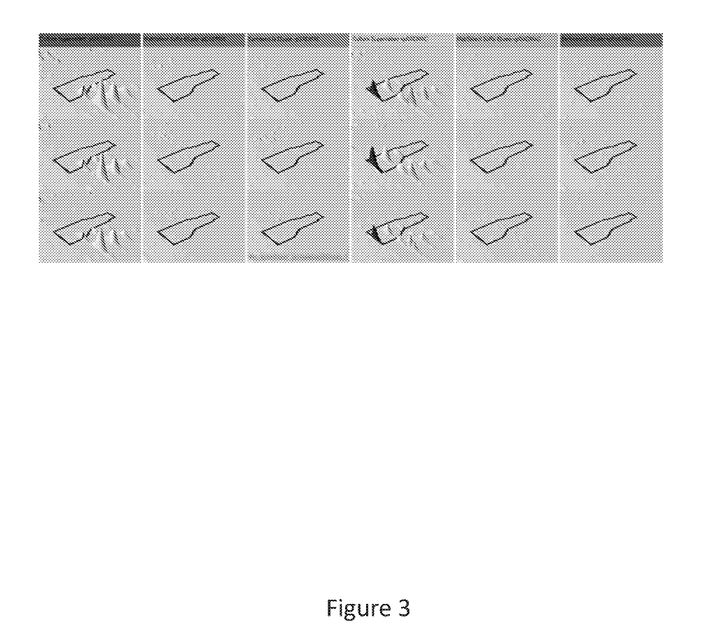

Ten proteins showed a significant (p<0.05, q<0.05) increase in pDADMAC-treated CCCS samples (Table 6). LC-MS profile for an example peptide used for quantitation of Protein SET (the protein showing the highest change in abundance) is shown in FIG. 3.

TABLE 6
HCPs Showing Significant (p < 0.05, q < 0.05) Increase
in Abundance in CCCS on pDADMAC Treatment.

| | | Abundance (ng/mg Total | | |
|---|---|---|---|---|
| | | Protein) | | |
| | | | CCCS + | Fold |
| Protein | Biological Relevance | CCCS | pDADMAC | Change |
| Protein SET | Apoptosis, transcription, | 7 ± 0 | 131 ± 9 | 18.7 |
| | nucleosome assembly and | | | |
| | histone chaperoning | | | |
| Nucleolin | Induces chromatin | 48 ± 4 | 322 ± 30 | 6.7 |
| | decondensation by binding | | | |
| | to histone H1 | | | |
| Acidic leucine-rich | Proliferation, | 10 ± 0 | 36 ± 7 | 3.6 |
| nuclear phosphoprotein | differentiation, apoptosis, | | | |
| 32 family member A | inhibition of protein | | | |
| | phosphatase 2A, regulation | | | |
| | of mRNA trafficking, | | | |
| | inhibition of | | | |
| | acetyltransferases as part of | | | |
| | the INHAT (inhibitor of | | | |
| | histone acetyltransferases) | | | |
| | complex | | | |
| Putative | DNA binding proteins that | 28 ± 1 | 92 ± 7 | 3.3 |
| uncharacterized protein | associates with chromatin | | | |
| By homology: High | and has the ability to bend | | | |
| mobility group protein, | DNA | | | |
| nuclear, DNA binding | | | | |
| Histone H2A.Z | Variant histone H2A which | 62 ± 2 | 114 ± 11 | 1.8 |
| | replaces conventional H2A | | | |
| | in a subset of nucleosomes | | | |
| Suprabasin | Unknown, upregulated in | 263 ± 9 | 429 ± 34 | 1.6 |
| | differentiating keratinocytes | | | |
| Insulin-like growth | Prolongs half-life of IGFs. | 107 ± 3 | 171 ± 11 | 1.6 |
| factor-binding protein 4 | Alter the interaction of IGFs | | | |
| | with their cell surface | | | |
| | receptors | | | |
| Corneodesmosin | Important for the epidermal | 33 ± 1 | 53 ± 3 | 1.6 |
| | barrier integrity | | | |
| Uncharacterized protein | One of the major pre- | 27 ± 2 | 41 ± 3 | 1.5 |
| By homology: | mRNA-binding proteins. | | | |
| heterogeneous nuclear | Can also bind poly(C) | | | |
| ribonucleoprotein K | single-stranded DNA. Plays | | | |
| isoform 4 | a role in p53/TP53 response | | | |
| | to DNA damage. | | | |
| Phospholipid transfer | Facilitates the transfer of a | 254 ± 14 | 307 ± 44 | 1.2 |
| protein | spectrum of different lipid | | | |
| | molecules | | | |

All values are Mean±Standard Error of the Mean

Across the analysis, no significant difference (p<0.05, q<0.05) was determined in the abundance of any HCPs in the protein A eluate samples or final product samples due to the presence or absence of pDADMAC (Table 7). The statistical approach taken here was based on the proposed application for the analytical method, which was to determine if an individual purification process generated significant improvement or degradation in performance compared to the overall population (and therefore used the null hypothesis that all the populations were equivalent). Therefore that the process that included pDADMAC was not significantly better or worse than the control process in terms of specific HCP levels.

TABLE 7
HCP Clearance through DSP for Proteins Showing Increased Abundance when
Cultures are Harvested using pDADMAC (MSS refers to MabSelect SuRe)

| | Abundance (ppm) ± SEM | | | | | |
|---|---|---|---|---|---|---|
| | | | | | MSS | SBQ |
| | | MSS | SBQ | CCCS + | Eluate + | Eluate + |
| Protein | CCCS | Eluate | Eluate | pDADMAC | pDADMAC | pDADMAC |
| Suprabasin | 263 ± 9 | 0 ± 0 | 1 ± 1 | 429 ± 34 | 1 ± 0 | 2 ± 1 |
| Phospholipid | 254 ± 14 | 3 ± 0 | 4 ± 1 | 307 ± 44 | 4 ± 1 | 4 ± 1 |
| transfer protein | | | | | | |
| Insulin-like | 107 ± 3 | 1 ± 0 | 0 ± 0 | 171 ± 11 | 0 ± 0 | 1 ± 0 |
| growth factor- | | | | | | |
| binding protein 4 | | | | | | |
| Histone H2A.Z | 62 ± 2 | 0 ± 0 | 0 ± 0 | 114 ± 11 | 0 ± 0 | 0 ± 0 |
| Nucleolin | 48 ± 4 | 3 ± 0 | 4 ± 0 | 322 ± 30 | 6 ± 1 | 6 ± 0 |
| Corneodesmosin | 33 ± 1 | 0 ± 0 | 0 ± 0 | 53 ± 3 | 0 ± 0 | 0 ± 0 |
| Putative | 28 ± 1 | 0 ± 0 | 0 ± 0 | 92 ± 7 | 0 ± 0 | 0 ± 0 |
| uncharacterized | | | | | | |
| protein | | | | | | |
| Uncharacterized | 27 ± 2 | 0 ± 0 | 1 ± 0 | 41 ± 3 | 0 ± 0 | 0 ± 0 |
| protein | | | | | | |
| Acidic leucine- | 10 ± 0 | 1 ± 0 | 1 ± 0 | 36 ± 7 | 0 ± 0 | 1 ± 0 |
| rich nuclear | | | | | | |
| phosphoprotein | | | | | | |
| 32 family | | | | | | |
| member A | | | | | | |
| Protein SET | 7 ± 0 | 0 ±0 | 0 ± 0 | 131 ± 9 | 0 ± 0 | 0 ± 0 |

All values are Mean ± Standard Error of the Mean

### Example 4: Process Immunogenicity Risk Assessment

On review of the HCP load in purified product samples, it was calculated that 3% of the HCPs identified in CCCS comprise greater than 60% of the HCP content in purified samples. This observation is consistent with previous work showing that “piggybacking” of HCPs through purification is likely to be responsible the bulk of HCP impurity (N. E. Levy, et al., “Identification and characterization of host cell protein product-associated impurities in monoclonal antibody bioprocessing,” Biotechnol. Bioeng. 111(5), 904 (2014); V. N. Sisodiya, et al., “Studying host cell protein interactions with monoclonal antibodies using high throughput protein A chromatography,” Biotechnol. J. 7(10), 1233 (2012); R. D. Tarrant, et al., “Host cell protein adsorption characteristics during protein A chromatography,” Biotechnol. Prog. 28(4), 1037 (2012)); and confirmed that a major difference in HCP population exists between that present in purified samples and that used for generation of the antisera used for an HCP ELISA.

The complete list of HCPs identified in CCCS samples was analysed using the Lonza Epibase® platform, resulting in a risk score assigned to each protein (the immunogenicity risk score, e.g., immunogenicity risk score). The resulting scores represent a worst-case scenario for T-cell activation—only a subset of the peptides identified as MHC-binders will actually be T-cell epitopes due to other factors contributing to immunogenicity such as protein internalization, antigen processing and T cell receptor specificity. The top 20 most abundant proteins in CCCS samples are shown in Table 8 along with their respective process immunogenicity risk scores.

TABLE 8
Immunogenicity Risk Scores for 20 Most Abundant HCPs in
Purified Product Harvested with and without pDADMAC.

| | Protein Immunogenicity Risk Score | |
|---|---|---|
| | (Protein Abundance × Immunogenicity Risk Score) | |
| Protein Number | SBQ Eluate | SBQ Eluate + pDADMAC |
| Basement membrane-specific | 3230725.2 ± 181647.2 | 2439262.4 ± 194622 |
| heparan sulphate proteoglycan | | |
| core protein | | |
| Chondroitin sulfate | 3496865.1 ± 316100.8 | 3437596.2 ± 592689 |
| proteoglycan 4 | | |
| Nidogen-1 | 593839.4 ± 26992.7 | 609263.8 ± 196661.1 |
| Laminin subunit beta-1 | 318492.4 ± 25923.8 | 370340 ± 66661.2 |
| Putative uncharacterized | 5326.2 ± 403.5 | 6859.5 ± 1129.8 |
| protein | | |
| Complement C3 | 666292.9 ± 67758.6 | 779223.9 ± 191982.7 |
| Clusterin | 177496 ± 9681.6 | 232358.4 ± 35499.2 |
| Thrombospondin 1 | 122155 ± 19989 | 124376 ± 17768 |
| Glutathione S-transferase Mu | 247497.3 ± 21063.6 | 221167.8 ± 31595.4 |
| 1-like protein | | |
| Alpha-enolase | 74562.4 ± 3389.2 | 74562.4 ± 5083.8 |
| 78 kDa glucose-regulated | 16455.6 ± 1175.4 | 16455.6 ± 1959 |
| protein | | |
| Galectin-3-binding protein | 203000.5 ± 16459.5 | 252379 ± 16459.5 |
| Putative phospholipase B-like 2 | 231035 ± 19803 | 310247 ± 52808 |
| Lysosomal alpha-glucosidase | 229591.8 ± 20258.1 | 276860.7 ± 13505.4 |
| Heat shock protein 84b | 244.8 ± 14.4 | 432 ± 43.2 |
| Dystroglycan | 97226.4 ± 11438.4 | 94366.8 ± 14298 |
| Endoplasmin | 28325.7 ± 2098.2 | 38816.7 ± 12589.2 |
| Uncharacterized protein | 0 ± 0 | 0 ± 0 |
| (Fragment) | | |
| Putative uncharacterized | 315724.8 ± 13155.2 | 263104 ± 39465.6 |
| protein | | |
| Tubulointerstitial nephritis | 76452 ± 6648 | 86424 ± 6648 |
| antigen-like | | |

An overall HCP immunogenicity risk score was calculated for each of the samples analysed (Table 9). This was calculated as follows:

Process Risk Score=Σ([Protein Abundance]×[Immunogenicity Risk Score])

TABLE 9
Overall Process Immunogenicity Risk Scores for In-Process and Purified mAb
(MSS refers to MabSelect SuRe)

| | | | | | MSS | SBQ |
|---|---|---|---|---|---|---|
| | | MSS | SBQ | CCCS + | Eluate + | Eluate + |
| | CCCS | Eluate | Eluate | pDADMAC | pDADMAC | pDADMAC |
| Process Risk | 703.1 ± 30.8 | 14.8 ± 1.6 | 12.6 ± 1.2 | 698.4 ± 68.6 | 13.6 ± 1.3 | 12.7 ± 2.1 |
| Score (divided | | | | | | |
| by 10⁶ for | | | | | | |
| readability | | | | | | |

purposes only)

The Process Risk Scores demonstrate in a biologically-relevant manner that the process change under investigation resulted in no increased patient risk based on the levels of immunogenic proteins. Additionally these scores were used to identify high-impact proteins in terms of immunogenicity that were unlikely to be required in suspension cell culture.

These results demonstrate the proteomic based analysis of HCPs can be used throughout the entire manufacturing process, thus avoiding development of different assays to detect HCPs during scalability of manufacturing, i.e., the method is independent of scale. The use of the same monitoring method throughout the entire manufacturing process from small scale to large scale avoids multiple issues such as the need to develop multiple monitoring methods at different manufacturing scales, along with concomitant errors in translation of the output from one scale to another scale. Rather, the proteomic method provides a simple, consistent, reproducible method that can be used independent of manufacturing scale. It also ensures that the same set of HCPs are monitored at different stages of the process to show the changes in HCPs, for example, during cell culture, protein expression, as well as before and after purification.

This process includes an immunogenicity calculation that is fully integrated into the development process, providing a number of advantages, including, but not limited to, (1) a method that allows immunogenicity to be performed for any production system where the genome is known, or for specific variants of the production system (eg GS CHO specifically as a subset of CHO), and (2) immunogenicity assessment to be performed for different patient populations (e.g. by geographic area or ethnicity). This is important as an overall average score for an HCP for the global population may mask a high score for a single particularly susceptible group.

In summary, the preceding examples demonstrate a rapid and scalable qualitative and semi-quantitative analysis of HCP impurities. This approach demonstrated an approximate 10-fold throughput increase compared to the most prevalent established method (C. E. Doneanu, et al., “Analysis of host-cell proteins in biotherapeutic proteins by comprehensive online two-dimensional liquid chromatography/mass spectrometry,” MAbs. 4(1), 24 (2012)). Analysis was performed using one dimensional reversed-phase nanoLC-MS² using a Tribrid mass spectrometer with an analysis time of less than 1 hour per sample. This approach relied on generation of a reference library of MS¹ peaks derived from HCPs determined from clarified culture supernatant. This reference library defines windows in retention time and m/z space that may then be applied to data acquired for purified samples. The ability of the test method to identify HCPs in the purified antibody therapeutic is therefore maximized and decoupled from the semi-quantitative analysis, performed using Hi3 methodology (J. C. Silva, et al., “Absolute quantification of proteins by LCMSE: a virtue of parallel MS acquisition,” Mol. Cell Proteomics. 5(1), 144 (2006)). The HCP profiles of purified material were then combined with the Epibase® in silico immunogenicity prediction tool to generate an overall HCP immunogenicity risk score for a production process. Once identified and assessed in terms of patient impact potential product impact or in vivo relevance, key HCPs of specific importance can then be monitored as part of routine QC analysis via specific ELISAs or targeted LC-MS² methods.

### Example 5: Methods

In Examples 5-8, a proteomic approach for both risk-based process development and mechanistic analysis of protein expression systems was tested in both mammalian and yeast systems. The method determined overall and specific risk factors to facilitate selection and optimisation of production processes, and delivered results within 1 week of sample receipt—turnaround of data being of critical importance in a process development environment. The data generated in this way was not obtainable by any other established technique.

This dataset establishes that routine use of this method can be implemented in commercial process development projects.

The following describes methods used and referenced throughout Examples 6, 7, and 8. Where Example 5 is silent, the methods of Example 1 apply to Examples 6, 7, and 8.

Sample Preparation for LC-MS/MS

Three replicates were prepared for each sample. 10 μl volumes of each sample were transferred into 0.5 ml Eppendorf tubes. 90 μl 0.5M MES (2-(N-morpholino)ethanesulfonic acid) pH 5.5, 6.6M guanidine HCl, 10 mM TCEP (Tris(2-carboxyethyl)phosphine) was added to each replicate. Incubation was performed at 50° C. for 30 minutes. All samples were subsequently buffer exchanged using ZebaSpin desalting columns (Thermo) into 0.1M Tris pH 8.0, 0.5M urea, 1 mM TCEP according to the manufacturer's instructions. Digestion was performed by addition of mass spectrometry-grade trypsin (Promega) to a 1:20 trypsin:protein ratio with incubation at 25° C. for 18 hours. Digestion was quenched by addition of 2% (final) TFA (trifluoroacetic acid).

LC-MS/MS Data Acquisition

Data was acquired using a Dionex RSSLnano nanoLC system coupled to a Thermo Fusion Tribrid Q-OT-qIT (Quadrupole-Orbitrap-Linear Ion Trap) mass spectrometer. A 1 μl volume of the tryptic peptides for each sample were injected onto a PepMap100 C18 300 Å Nano-Trap column (Thermo) in a loading buffer of 98:2 water:acetonitrile plus 0.08% TFA at 12 μl/min for 3 minutes. After 3 minutes the nanoLC flow was directed in the reverse direction through the trapping column onto the analytical column (EasySpray RSLC C18 2 μm, 100 Å, 75 μm×25 cm (Thermo)). A linear gradient was applied between 0.1% formic acid in water and 0.1% formic acid in 80:20 acetonitrile:water.

Source ionisation settings were static during the acquisition at 2500 V spray voltage and a transfer tube temperature of 275° C. The mass spectrometer was configured in positive ionisation mode for acquisition of MS¹ data in the orbitrap at 120,000 FWHM nominal resolution with quadrupole isolation over a range of 350-1,550 m/z, an AGC target of 2.0e5 and a maximum injection time of 50 ms. Data was mass-corrected using an internal standard based on a flouranthine ion lockmass at generated from a separate reagent ion source. Only charge states between z=2 and z=6 were selected for MS² fragmentation.

MS² fragmentation was performed in the linear ion trap at normalised collision energy of 28%, an AGC target of 1.0e4 and a maximum scan time of 200 ms at the “Normal” trap scan rate.

Mass Spectrometry Data Analysis

Protein identifications based on MS² fragmentation were performed using PEAKS Studio software. Protein identification was performed for CCCS (clarified cell culture supernatant) only. False discovery rate at the peptide level was controlled at <0.1% using decoy fusion methodology (Zhang, J, et al., “PEAKS DB: de novo sequencing assisted database search for sensitive and accurate peptide identification”, Mol. Cell Proteomics 4(11), 111 (2012)). At least 3 unique peptides were required for each protein assignment. Mass tolerances were specified at <7.5 ppm for parent ions and <0.3 Da for fragment ions.

Data was processed against the UNIPROT CHO proteome (http://www.uniprot.org/) using PEAKS studio 7 to identify the proteins present. Identification was performed only for culture supernatant samples. All samples were processed using Progenesis QI for Proteomics. Identifications from PEAKS were imported into Progenesis for quantitation throughout the experiment. Data for each cell line was processed independently. Quantitation was performed using Hi5 methodology.

For each of the SBQ (Sartobind Q) purified cell lines, Hi5 quantitation was performed within Progenesis QI for Proteomics software. In this case, the Hi5 quantitation was performed within the software (which was upgraded to include this functionality)—this capability greatly decreased the complexity of data analysis. For quantitation purposes, either the antibody heavy or light chain was used as the quantitation standard and was set as 2,000,000 (the units in the software are specified as fmol, but the value was set such that HCP levels are reported as parts per million, since each mol of mAb contains 2 mol of heavy chain and 2 mol of light chain).

In Silico Immunogenicity Analysis

Sequences for each identified protein were submitted for in silico immunogenicity assessment using the Epibase® platform.

Epibase® is an in silico platform for immunogenicity risk screening. The platform identifies potential T cell epitopes in a protein sequence by predicting the binding affinities of all 10-mer peptides derived from the sequence to HLA class II receptors.

The screening was performed targeting the Global population. The Global population HLA set includes 85 HLA class II allotypes, in particular 43 DRB1 allotypes which are a primary focus of immunogenicity profiling. A human proteome filter, including the top 25% most abundant human proteins, was used to filter-out self-peptides (peptides which are presented on HLA molecules but will not bind T cell receptors).

The immunogenicity risk score for a protein is obtained by taking into account the number of predicted T cell epitopes in a protein and population frequencies of affected HLA allotypes.

### Example 6: Selection of Host Cell Line and Purification Conditions to Minimize Presence of High Risk Proteins

The methods provided in this example are of use in development of purification schemes for biologics. A typical workflow for optimisation of a purification process step involves screening combinations of conditions. In this case an ion exchange step using Sartobind Q resin was investigated in terms of the buffer pH used in the procedure and the loading capacity of the column. Some established approaches use AKTA-scale purifications to optimise these conditions, resulting in a comparison of 9 samples in total. However, increased use of robotic platforms for screening of purification conditions has resulted in a substantial increase in sample numbers that will be generated in standard development stages. To support these stages, analysis of 96 or more samples in a single analysis must be performed with the appropriate turnaround speed of results to allow decision-making.

To mimic these conditions, additional comparisons between host cell lines and products were also included in the experiment. This allowed a representative sample number to be tested, but also demonstrated that the method could support integrated testing of candidate production cell lines and production schemes. It was hypothesised that cell lines expressing the same product could have very different impurity profiles/risks, and that selection could be based on this parameter in addition to existing measures of titre, product quality and growth characteristics. In this case, it would be expected that these cell lines would also have different optimal conditions for purification.

The factors investigated in this experiment were as follows:
Purification buffer pH: pH5, pH 6 and pH 7
Loading capacity: 500 mg/ml, 1000 mg/ml, 1500 mg/ml
   These factors were investigated for the following cell lines and products:
E22, a cell line expressing cB72.3 antibody which grows to high cell concentrations in the Lonza platform process
3C12, a cell line expressing cB72.3 antibody which grows to average cell concentrations in the Lonza platform process
A14, a cell line expressing H35k1 antibody which grows to average cell concentrations in the Lonza platform process

Every combination of these factors was analysed as three technical replicates, generating 81 samples in total. This sample number was similar to that deriving from DoE (Design of Experiments) experimental approaches (however in DoE designs only one technical replicate would be performed, since the replication would be already integrated into the experimental design). Furthermore, culture supernatant samples for each cell line were also analysed in triplicate—these were used for detection, identification of HCPs and to generate the impurity maps that would be applied to the purified samples for quantitation of individual HCPs.

The list of identified HCPs was then analysed by the previously reported Epibase® in silico immunogenicity assessment tool, but also by gene ontology to assess potential interaction with the drug product. This demonstrates that this same methodology can be used for any risk-based tool that is able to generate a numerical factor from a list of protein identifiers or sequences. In the case of antibody production, impact of HCP immunogenicity could be considered relatively low, since Protein A affinity purification is a highly effective method for impurity removal. However, there have been several reported cases where HCPs have caused issues in terms of stability of drug product because of interactions either with the biotherapeutic itself or other surfactants.

Product interaction risk scores were determined by looking for relevant keywords in gene ontology terms. These terms derive from database searches against the gene identifiers found within the test samples. Proteins with terms relating to protease activity were tagged as relevant and given a score of 1. Specific proteins with documented activity in degradation of drug products (either protein or surfactant degradation) were given an additional higher weighting of 9 (in this case, Cathepsin D¹ and Phospholipase B²). Development of a wider database, including other proteins that have been empirically shown to effect drug stability, efficacy or safety would expand this approach, and scoring can be made specific to the therapeutic protein or formulation. For example, Phospholipase B would only be considered relevant for products with formulations containing polysorbate.

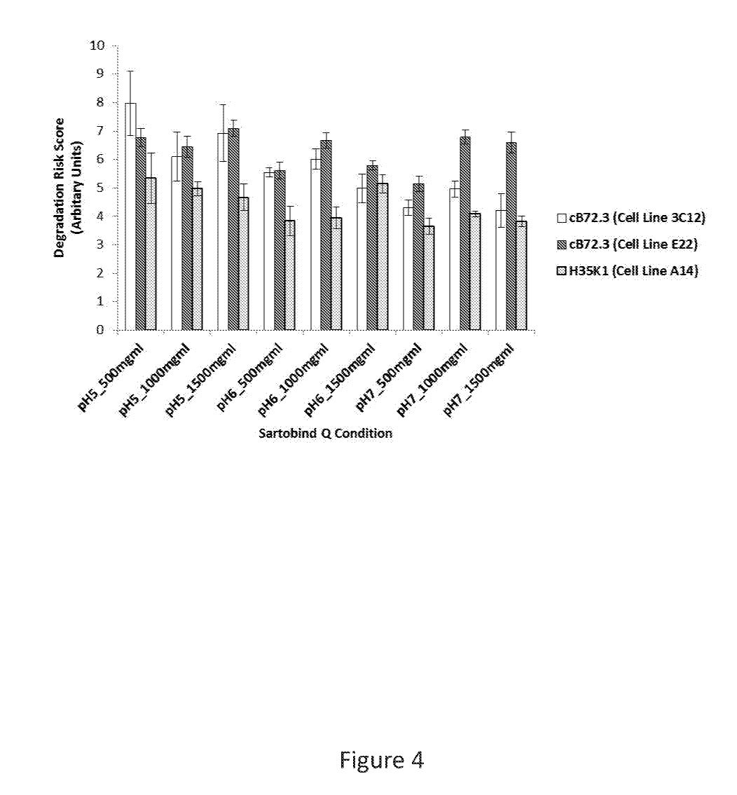

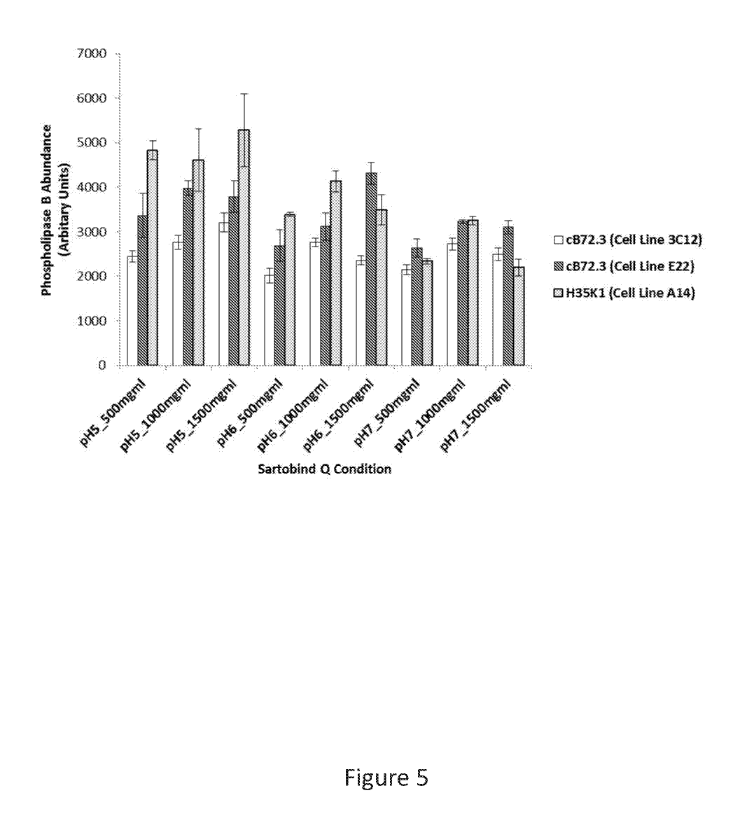

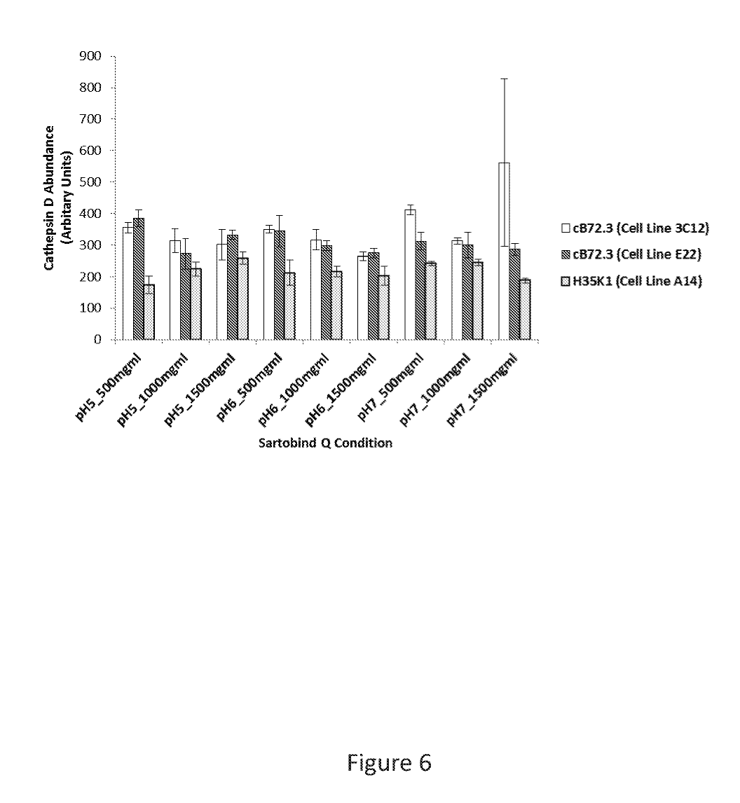

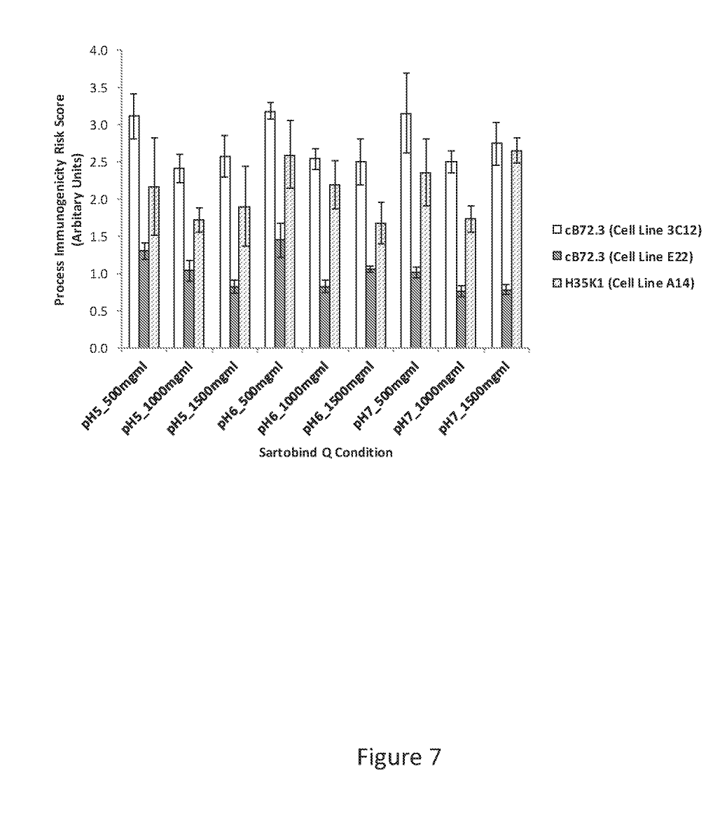

This scoring allowed selection of specific process conditions that carried the least risk for degradation of the final drug product. Aggregated product interaction risks for the different cell lines and purification conditions are shown in FIG. 4, profiles for specific HCPs reported to have negative consequences on drug product stability are shown in FIG. 5 and FIG. 6. Immunogenicity assessment based on HCP amino acid sequence analysis with Epibase® is shown in FIG. 7.

Significant differences between cell lines and purification conditions could be clearly discriminated for both general and specific product interaction risks as well as for immunogenicity assessment.

A change in HCP profile was noted for the two cell lines expressing the same cB72.3 product, indicating this this approach can also be used to select cell lines based on HCP impurity profile, as well as between purification schemes. For the two cell lines expressing the cB72.3 product, E22 gave a higher product degradation risk score compared to 3C12 under most of the conditions tested. A general trend was observed that increased loading capacities decreased product degradation risk, however individual proteins of interest exhibited individual response to the conditions. For example, phospholipase B level increased with loading capacity at low pH for cell line A14.

This approach was therefore demonstrated to detect differences in risk between test samples to facilitate informed decision making based on individual and aggregated risk factors for each specific protein therapeutic.

Overall recommendations that would be made for purification strategies would be as follows:

E22—pH 7 at 500 mg/ml
3C12—pH 7 at any candidate loading capacity
H35K1—pH 7 at any candidate loading capacity

However, recommendations may change depending on desired formulation (based on phospholipase B interaction with excipients) and administration route (e.g. subcutaneous administration may require a higher weighting for immunogenicity risk factors).

The method could also be used for selection of cell lines—in this case, 3C12 was demonstrated to generate lower levels of HCPs in purified samples that could impact drug product stability than E22 under all condition tested, despite being derived from the same host cell line and making the same product. Conversely E22 consistently generated purified samples with lower overall HCP immunogenicity risks in comparison to 3C12.

### Example 7: Assessment of Different Processes for Recovery of Product

Application of proteomic methods to development of primary recovery processes was also evaluated. In this case, two alternative methods for pre-clearance of HCPs before protein A chromatography were tested: chromatin extraction following the approach of Nian, R. et al “Advance chromatin extraction improves capture performance of protein A affinity chromatography”, J Chromatogr A, 1431, 1-7 (2016) and clarification of supernatant using 3M Emphaze filters. A sample of the culture supernatant from a culture expressing cB72.3 antibody that was used for both methods was also analysed to generate the HCP impurity maps that were applied to the test samples for quantitation. All samples were analysed in triplicate.

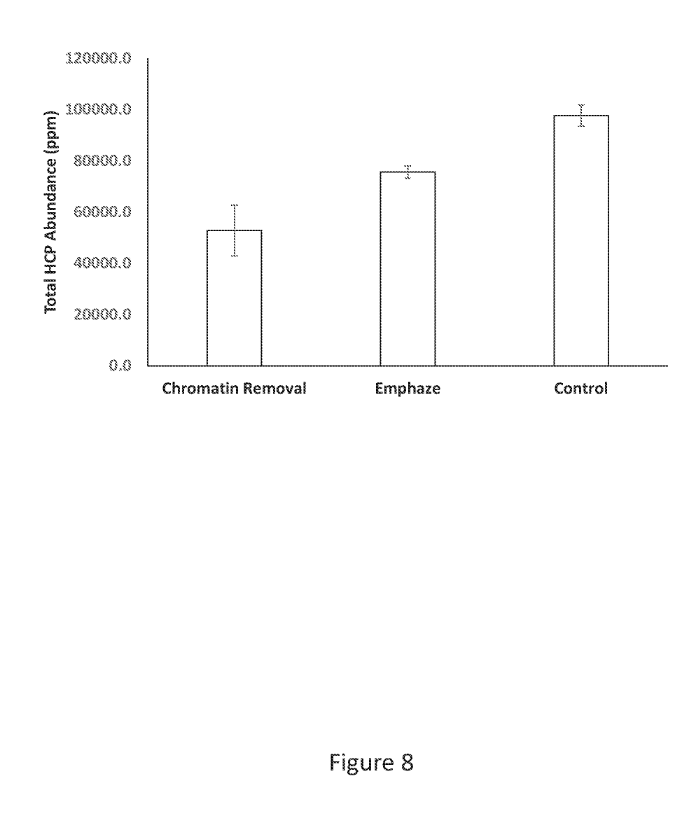

Both treatment methods reduced the HCP load in each respective sample in comparison to the control sample. The chromatin removal treatment reduced the overall HCP load to approximately half of that in the control. The Emphaze treatment reduced the overall HCP load by approximately 25% (FIG. 8). Reduction of HCP load was not equivalent for each protein—some proteins were unaffected by either treatment. Chromatin removal was generally more effective the Emphaze—no proteins were reduced more by Emphaze than by the chromatin removal process. Additional analysis of the biophysical properties of each HCP could be performed to determine if there are specific properties that influence

Risk analysis for the different process alternatives was performed against potential for product interaction as described previously. In addition, an additional process risk was assessed—product dissociation (reduction of antibody inter-chain disulphide bonds) has been observed in many processes and products across the industry. This has been linked to the presence of two specific enzyme systems, the thioredoxin and glutaredoxin systems, in culture supernatant. In this experiment, dissociation risk was assessed by scoring both these documented enzyme systems in terms of each potential gene product that could participate as well as other proteins that have reductive potential, such as those that are involved in disulphide bond isomerisation.

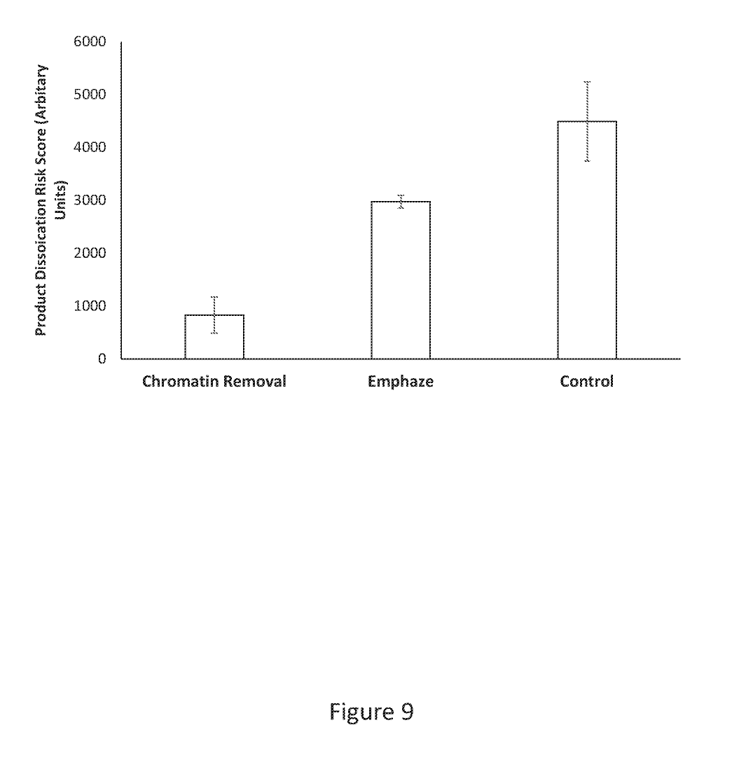

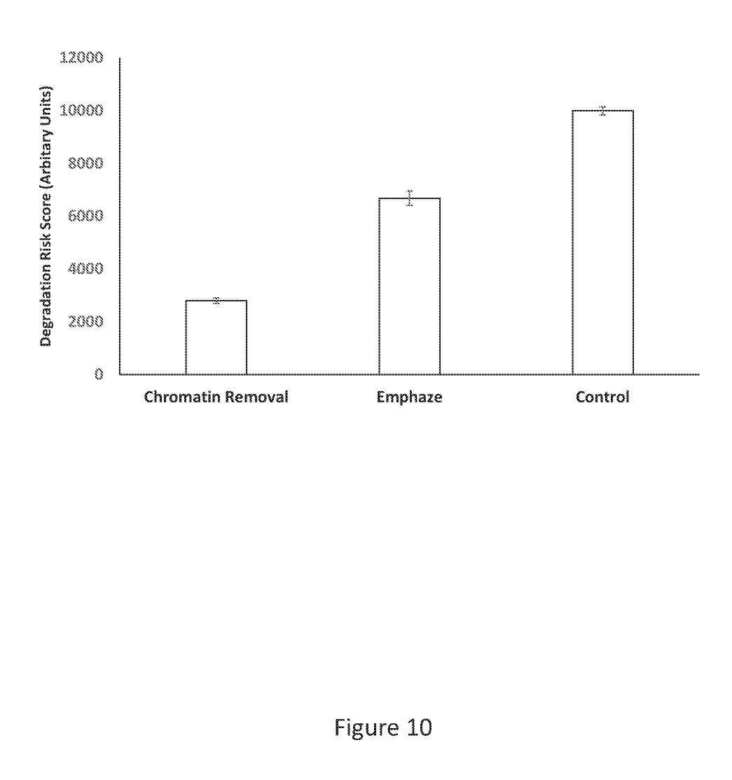

Risk assessments of the different process options showed that in each case, the general trend for risk-tagged proteins was the same as for total HCP. However, the dissociation risk scoring showed a larger-scale reduction for the chromatin reduction treatment than the general level of HCP reduction—a 5-fold reduction in dissociation risk score (p<0.03) in comparison to a less than 2-fold reduction in total HCP (FIG. 9). Degradation risk score followed a similar pattern, in that risk was reduced by the chromatin removal treatment more than would be predicted from total HCP level (FIG. 10).

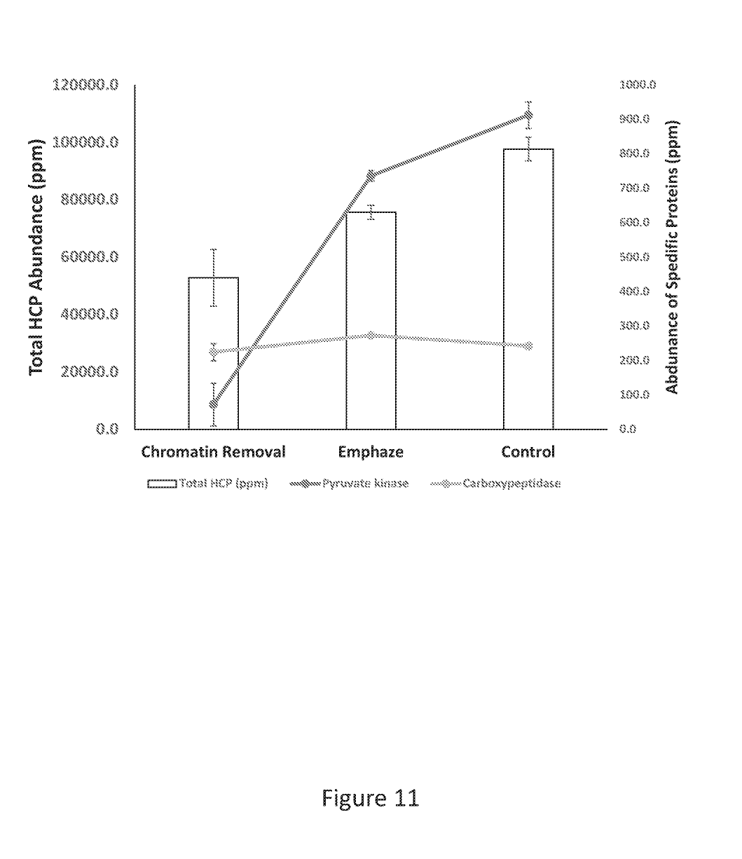

The differences observed between total protein and specific risk scores highlight the gain in information generated by monitoring HCPs individually rather than by the traditional aggregate measurement and how that information can be used to drive process decisions. Many proteins in the same dataset showed no difference in abundance between treatments—if any of these proteins were identified as a risk factor, overall measurement of total HCP would be misleading when engineering processes to remove them. For example, carboxypeptidase (gene name I79_006816) showed no change across any of the samples, while pyruvate kinase (gene name H671_4g13041) was unaffected by Emphaze treatment, but almost completely depleted by the chromatin removal treatment (FIG. 11).

### Example 8: Assessment of Different Expression and Fermentation Systems for Recovery of Product

Cell culture supernatant samples from Pichia pastoris expression system were analysed for HCP content and composition. The samples represented three different reporter proteins, two different induction system and different fermentations conditions with varying pH and temperature as shown in Table 10. Each sample was tested in triplicate.

TABLE 10
Sample Details for Pichia HCP Analysis

| Sample | Reporter | | Temperature | Titre | |
|---|---|---|---|---|---|
| Number | Protein | pH | (° C.) | (mg/L) | Comments |
| 1 | Protein 1 | 5.0 | 30 | 250 | N/A |
| 2 | Protein 1 | 6.0 | 30 | 600 | N/A |
| 3 | Protein 1 | 6.8 | 30 | 700 | Possible contamination |
| 4 | Protein 1 | 6.0 | 25 | 500 | N/A |
| 5 | Protein 2 | 4.0 | 25 | 1300 | N/A |
| 6 | Protein 2 | 6.0 | 30 | 1500 | N/A |
| 7 | Protein 3 | 5.0 | 25 | 3000 | pAOX induction |
| 8 | Protein 3 | 5.0 | 25 | 3000 | pAOX induction |
| 9 | Protein 3 | 5.0 | 25 | 6000 | pG1.3 induction |
| 10 | Protein 3 | 5.0 | 25 | 4000 | pG1.3 induction |
| 11 | Protein 3 | 5.0 | 25 | 6500 | pG1.3 induction |

Generated LC-MSMS data were analysed separately for each of the therapeutic protein to allow better alignment of the data during processing. Database searching was performed against the proteome for Komagataella phaffii (strain ATCC 76273/CBS 7435/CECT 11047/NRRL Y-11430/Wegner 21-1) (Yeast) (Pichia pastoris) from UniProt database. Proteins displaying a significant change in expression profile (q value <0.01) and fold change >2 were evaluated for similarity in expression profile.

pAOX Induction

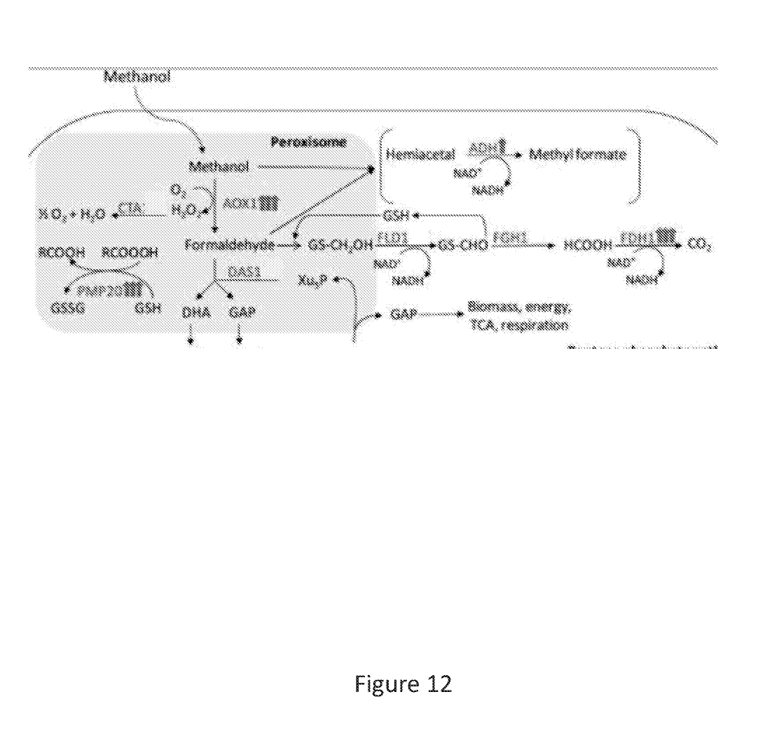

Protein 3 was expressed using methanol induction system pAOX and Lonza's limited glucose induction system pG1.3. Samples incorporating the pAOX system displayed significant increases in the abundance of proteins specific to methanol metabolism (alcohol oxidase, formate dehydrogenase, alcohol dehydrogenase) as well as metabolism of reactive oxygen species which are generated during methanol metabolism (superoxide dismutase, peroxiredoxin PMP, protein disulphide-isomerase, thioredoxin). Metabolic processes surrounding methanol metabolism are shown in FIG. 12, along with the observed changes in protein expression levels in the pAOX induced cultures in comparison to pG1.3 induction.

Additional changes were observed within this experiment, correlating with the high titres for samples 9 and 11 in comparison to samples 7, 8, and 10. These proteins included ATPases involved in protein folding, GPI-anchored cell surface glycoprotein, Peptidyl-propyl cis trans isomerase and Uncharacterized protein F2QUJ0 which shows homology to Translation elongation factor EF-1 gamma (Komagataella phaffii) (C4R6E8).

Samples representing pG1.3 system also displayed increased expression of enzymes involved in procession carbohydrates (glucanase, glucosidase) as well as structural proteins.

Temperature and pH Effects on Supernatant Proteins

Varying fermentation conditions corresponded to changes in protein expression profiles. Increased abundance was observed for the chaperone protein HSP90 for fermentation runs at increased temperature and pH.

Condition 3 showed a major increase in abundance of wide range of proteins including proteins involved with protein biosynthesis e.g. ribosomal proteins, elongation factor; cellular respiration e.g. dehydrogenases, ATP synthase, mitochondrial proteins. This correlated with an observation during this process that a contamination event may have occurred. Identification of an increased number of proteins in this sample demonstrates that this method is also capable of detecting where growth of other organisms in bioreactors has perturbed the protein composition of the culture supernatant.

In this example, the proteomic method was used to perform a more mechanistic analysis of an expression platform, rather than a risk-based assessment for process selection. This analysis of Pichia Pastoris samples generated information on the metabolic processes involved in induction of protein expression and changes in culture conditions. This technology enables optimisation of bioprocesses for production of chaperones, folding proteins and systems for post-translational modification of proteins, all of which are relevant to successful production of biotherapeutics.

Collectively, these examples show methods useful to rapidly analyze hundreds of samples to identify and assess hundreds of possible protein, e.g., HCP, contaminants introduced in the processes and methods of manufacturing. The methods shown herein have the ability to evaluate different processes and methods of manufacturing of products, e.g., recombinant polypeptides, and select between said processes based on the risk scores associated with the protein, e.g., HCP, contaminants they produce, as well as the overall risk scores associated with the processes and methods of manufacturing themselves. The methods shown herein have the ability to monitor a given process or method of manufacturing from early stages, e.g., cell supernatant, through the final purified product, e.g., recombinant polypeptide or purified therapeutic product.

What is claimed is:

1. A simple method of rapidly analyzing a sample, e.g., to provide an assessment of the risk of a protein (e.g., the risk the protein presents if present as a contaminant in a preparation, e.g., a preparation to be administered to a subject, e.g., a pharmaceutical preparation), the method comprising:
a) providing, e.g., forming and/or maintaining, a sample mixture comprising:
i) the sample, which comprises the protein and optionally a product (e.g., a recombinant polypeptide, e.g., an antibody, enzyme, or cytokine), produced via a process; and
ii) a denaturant, e.g., deoxycholate and urea,
under conditions, e.g., concentrations of denaturant, that denatures the protein in the sample at temperature of between 10 and 30° C., e.g., 18-26° C., e.g., 20±3° C., 20±2° C., 20±1° C., or 20° C.;
b) providing, e.g., forming and or maintaining, a sample/enzyme mixture comprising:
i) sample mixture (e.g., an aliquot of sample mixture from (a)); and
ii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;
c) separating the protein digestion products using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products; and
d) assigning a protein risk score to a protein identified in the sample,
thereby analyzing the sample and providing an assessment of the risk of a protein.

2. A simple method of rapidly analyzing a sample to provide an assessment of the risk of a protein, the method comprising:
a) providing, e.g., forming and/or maintaining, a sample mixture comprising:
i) the sample, which comprises the protein (e.g., a HCP) and optionally a therapeutic product (e.g., a recombinant polypeptide), produced via a process; and
ii) a first denaturant, e.g., guanidine hydrochloride,
under conditions, e.g., concentrations of a first denaturant, that denatures the protein in the sample at temperature of between 30 and 60° C., e.g., 45-55° C., e.g., 50±3° C., 50±2° C., 50±1° C., or 50° C.;
b) providing, e.g., forming and or maintaining, a sample/enzyme mixture comprising:
i) sample mixture;
ii) a second denaturant, e.g., urea; and
iii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;
c) separating the protein digestion products using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products;
d) assigning a protein risk score to a protein identified in the sample, thereby analyzing the sample and providing an assessment of the risk of a protein.

3. The method of either of claim 1 or 2, further comprising evaluating a plurality of different samples, each made by a different process, e.g., evaluating at least 2, 4, 8, 10, 50, 96, 100, 192, 200, 500 or 1,000, different samples.

4. The method of claim 3, further comprising comparing the assessment of risk for a first and a second different sample.

5. The method of claim 4, further comprising, responsive to the comparison, selecting, a process for producing the product.

6. The method of claim 4, further comprising, responsive to the comparison, selecting, classifying, or further processing one of the samples.

7. A method of evaluating a process of making a product, e.g., an evaluation that incorporates assessment of the risk presented by a protein other than the product, e.g., a contaminant, produced by the process, comprising:
a) providing, e.g., forming and/or maintaining, a sample mixture comprising:
i) the protein and optionally a product (e.g., a recombinant polypeptide, e.g., an antibody, enzyme, or cytokine), produced by the process; and
ii) a denaturant, e.g., deoxycholate and urea
under conditions, e.g., concentrations of denaturant, that denatures the protein in the sample, e.g., at temperature of between 10 and 30° C., e.g., 18-26° C., e.g., 20±3° C., 20±2° C., 20±1° C., or 20° C.;
b) providing, e.g., forming and or maintaining, a sample/enzyme mixture comprising:
i) sample mixture (e.g., an aliquot of sample mixture from a)); and
ii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;
c) separating the protein digestion products, e.g., by using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products; and
d) assigning a protein risk score to a protein identified in the sample,
thereby evaluating a process of making a product, e.g., an evaluation that incorporates assessment of the risk presented by a protein other than the product, e.g., a contaminant, produced by the process.

8. A method of evaluating a process of making a product, e.g., an evaluation that incorporates assessment of the risk presented by a protein other than the product, e.g., a contaminant, produced by the process, comprising:
a) providing, e.g., forming and/or maintaining, a sample mixture comprising:
i) the protein and optionally a product (e.g., a recombinant polypeptide, e.g., an antibody, enzyme, or cytokine), produced by the process; and
ii) a first denaturant, e.g., guanidine hydrochloride,
under conditions, e.g., concentrations of denaturant, that denatures the protein in the sample, e.g., at temperature of between 30 and 60° C., e.g., 45-55° C., e.g., 50±3° C., 50±2° C., 50±1° C., or 50° C.;
b) providing, e.g., forming and or maintaining, a sample/enzyme mixture comprising:
i) sample mixture (e.g., an aliquot of sample mixture from a));
ii) a second denaturant, e.g., urea; and
iii) an enzyme preparation comprising an enzyme for which the protein is a substrate, e.g., a proteolytic enzyme, e.g., an enzyme which cleaves proteins at a preselected or defined target site, e.g., trypsin, lysC, GluC, or AspN, with the sample mixture,
under conditions in which the enzyme maintains substantial activity and reacts with, e.g., cleaves, the protein to provide protein digestion products;
c) separating the protein digestion products, e.g., by using chromatography, e.g., 1-dimensional chromatography, providing the identity of the protein digestion products, e.g., by mass spectrometry, e.g., LC/MS, and using one or more protein digestion products to provide the identity of a protein associated with the protein digestion products; and
d) assigning a protein risk score to a protein identified in the sample,
thereby evaluating a process of making a product, e.g., an evaluation that incorporates assessment of the risk presented by a protein other than the product, e.g., a contaminant, produced by the process.

9. The method of either of claim 7 or 8, further comprising evaluating a plurality of different processes of making a product, e.g., evaluating at least 2, 4, 8, 10, 50, 96, 100, 192, 200, 500 or 1,000, different processes.

10. The method of claim 9, further comprising comparing the evaluation for a first and a second different process.

11. The method of claim 10, further comprising, responsive to the comparison, selecting a process of making the product.

12. The method of any of claims 1-11, wherein the protein is a contaminant or other undesirable component (e.g., a fragment, denatured, or mis-folded version of a product being produced by a set of conditions, or a host cell protein (HCP) or fragment thereof).

13. The method of any of claims 1-12, wherein the denaturant, first denaturant, or second denaturant comprises, consists of, or consists essentially of deoxycholate and urea, guanidine hydrochloride, or urea and guanidine hydrochloride.

14. The method of any of claims 1-13, wherein the concentration of denaturant in the sample mixture is higher than the concentration of denaturant in the sample/enzyme mixture.

15. The method of any of claims 1-14, wherein:
the concentration of denaturant in sample mixture is sufficiently high to denature the protein, e.g., wherein at least 50, 60, 70, 80, 90, 95, 96, 97, 98, 99, or 100% of the protein is denatured; and
the concentration of denaturant in sample/enzyme mixture is sufficiently low to not denature the enzyme, e.g., wherein less than 50, 40, 30, 20, 10, 5, 4, 3, 2, or 1% of the enzyme is denatured.

16. The method of any of claims 1-15, wherein the concentration of the denaturant in the sample mixture is:
i) at least 1, 1.5, 2, 2.5, 3, 3.5, 4, 4.5, 5, 5.5, 6, 6.5, 6.6, 6.7, 6.8, 6.9, 7, 7.5, or 8;
ii) 1-10 M, 2-9 M, 3-8 M, 4-7 M, 5-7 M, 6-6.6 M, 6 M, 6.6 M, or 8 M;
iii) 0-10 M, 2-9 M, 3-8 M, 4-7 M, 5-7 M, 0.5-5 M, 0.5-2 M, 0.5 M, 1 M, or 2 M; or
iv) 0.01%-50%, 1%-40%, 1%-20%, 0.5%-10%, 0.01%-5%, or 0.1%-2% (m/v).

17. The method of any of claims 1-16, wherein the sample mixture comprises a first denaturant (i.e. the denaturant of (a)(ii)), and the sample/enzyme mixture comprises the first denaturant and a second denaturant.

18. The method of claim 17, wherein the first denaturant is guanidine hydrochloride and the second denaturant is urea.

19. The method of either of claim 17 or 18, wherein the concentration of the first denaturant in the sample mixture is 1-10 M, 2-9 M, 3-8 M, 4-7 M, 5-7 M, 6-6.6 M, 6 M, 6.6 M, or 8 M.

20. The method of any of claims 17-19, wherein the concentration of the first denaturant in the sample/enzyme mixture is:
i) 0-10 M, 2-9 M, 3-8 M, 4-7 M, 5-7 M, 0.5-5 M, 0.5-2 M, 0.5 M, 1 M, or 2 M;
ii) less than or equal to 4, 3.5, 3, 2.5, 2, 1.5, 1, 0.5, or 0.1 M; or
iii) ii) less than or equal to 0.5 or 0.1 M, e.g., essentially 0 M.

21. The method of any of claims 17-20, wherein the concentration of the second denaturant in the sample/enzyme mixture is:
i) less than or equal to 4, 3.5, 3, 2.5, 2, 1.5, 1, 0.5, or 0.1 M;
ii) less than or equal to 0.5 or 0.1 M, e.g., essentially 0 M; or
iii) less than or equal to 2 M or 0.5 M.

22. The method of any of claims 1-21, wherein:
the pH of the sample mixture is sufficiently low that deamidation reactions are substantially inhibited e.g., wherein at least 50, 60, 70, 80, 90, 95, 96, 97, 98, 99, or 100% of the asparagine and glutamine side chains of the protein are unaltered, and
the pH of the sample/enzyme mixture is sufficiently high that the enzyme is active, e.g., wherein the enzyme is at least 50, 60, 70, 80, 90, or 100% active, e.g., operating at 50, 60, 70, 80, 90, or 100% efficiency compared to maximum efficiency.

23. The method of any of claims 1-22, wherein the pH of the sample mixture is 5.5±1, 0.75, 0.5, or 0.25 (e.g., 5.5±0.5) and the pH of the sample/enzyme mixture is 7.3±1, 0.75, 0.5, or 0.25 (e.g., 7.3±0.5).

24. The method of any of claims 1-23, wherein the pH of the sample mixture is 5.5 and the pH of the sample/enzyme mixture is 7.3.

25. The method of any of claims 1-24, wherein the method does not comprise alkylation of cysteine residues of the protein or protein digestion products.

26. The method of any of claims 1-25, wherein the sample mixture and/or sample/enzyme mixture comprise a reducing agent, e.g., Tris(2-carboxyethyl)phosphine (TCEP), dithiothreitol (DTT), or beta-mercaptoethanol.

27. The method of claim 26, wherein the concentration of reducing agent, e.g., Tris(2-carboxyethyl)phosphine (TCEP), dithiothreitol (DTT), or beta-mercaptoethanol, in the sample mixture is higher than in sample/enzyme mixture.

28. The method of either of claim 26 or 27, wherein:
the concentration of reducing agent, e.g., Tris(2-carboxyethyl)phosphine (TCEP), dithiothreitol (DTT), or beta-mercaptoethanol, in the sample mixture is sufficiently high to substantially reduce the cysteines of the protein, e.g., wherein at least 50, 60, 70, 80, 90, 95, 96, 97, 98, 99, or 100% of the cysteine residues of the protein are reduced; and
the concentration of reducing agent, e.g., Tris(2-carboxyethyl)phosphine (TCEP), dithiothreitol (DTT), or beta-mercaptoethanol, in the sample/enzyme mixture is sufficiently low to not interfere with other steps of the method or method of manufacturing, e.g., wherein the reducing agent does not significantly accumulate in equipment (e.g., mass spectrometer or analytical column) or produce additional signal in data (e.g., mass spectrometry data).

29. The method of any of claims 26-28, wherein the concentration of the reducing agent in the sample mixture is at least 1, 2, 3, 4, 5, 6, 7, 8, 9, 10, 11, 12, 13, 14, 15, 16, 17, 18, 19, or 20 mM, e.g., 1, 2, 3, 4, 5, 6, 7, 8, 9, 10, 11, 12, 13, 14, 15, 16, 17, 18, 19, or 20 mM.

30. The method of any of claims 26-29, wherein the concentration of the reducing agent in the sample/enzyme mixture is less than or equal to 10, 9, 8, 7, 6, 5, 4, 3, 2, 1, 0.5, or 0.1 mM, e.g., 10, 9, 8, 7, 6, 5, 4, 3, 2, 1, 0.5, or 0.1 mM.

31. The method of any of claims 26-30, wherein the reducing agent is Tris(2-carboxyethyl)phosphine (TCEP).

32. The method of any of claims 1-31, wherein the protein digestion products are separated on the basis of one or more (e.g., one, two, three or more) of size, charge, or affinity.

33. The method of any of claims 1-32, wherein separating the protein digestion products comprises using chromatography, e.g., 1-dimensional chromatography, e.g., affinity chromatography, gel filtration chromatography, ion exchange chromatography, reversed phase chromatography, hydrophobic interaction chromatography, high performance liquid chromatography (HPLC), gas chromatography (GC), capillary electrophoresis, ion mobility, or any chromatographic method described herein.

34. The method of any of claims 1-33, wherein (c) further comprises providing the identity of a protein digestion product, e.g., by mass spectroscopy, e.g., LC/MS, tandem mass spectrometry, or RP-LCMS².

35. The method of any of claims 1-34, wherein (c) comprises separating the protein digestion products using chromatography, e.g., 1-dimensional chromatography, and providing the identity of the protein digestion products, e.g., by mass spectrometry, e.g., LC/MS, tandem mass spectrometry, or 1D RP-LCMS².

36. The method of any of claims 1-35, wherein a plurality, e.g., at least 10, 20, 30, 40, 50, 60, 70, 80, 90, or 100% of protein digestion products in at least 2, 10, 20, 96, 100, 192, 1,000, or 10,000 samples are classified or assigned an identity, structure or composition.

37. The method of any of claims 1-36, wherein the protein risk score is a function of one or more of:
an unwanted, e.g., off-target, property in a subject to receiving a preparation comprising the protein and, optionally, a product, e.g., immunogenicity;
an unwanted effect of the protein in a preparation of the product, e.g., a preparation of a drug, e.g., the propensity to cause denaturation, precipitation, or color; and
a value for the abundance of the protein present in the sample.

38. The method of any of claims 1-37, wherein step (d) is repeated to provide a protein risk score for one or more (e.g., at least 2, 10, 50, 100, 200, 500, 1000, or all) proteins identified in the sample.

39. The method of any of claims 1-38, wherein step (d) comprises providing a protein risk score, e.g., an immunogenicity risk score, e.g., as generated by the Epibase® platform.

40. The method of any of claims 1-39, wherein step (d) comprises providing a immunogenicity risk score as generated by the Epibase® platform.

41. The method of any of claims 1-40, further comprising providing a process risk score to the sample.

42. The method of claim 41, wherein the process risk score is a function of the one or more protein risk scores of the sample's proteins.

43. The method of either of claim 41 or 42, wherein the process risk score is calculated based upon the formula:
Process Risk Score=Σ([Protein Abundance]×[Immunogenicity Risk Score]).

44. The method of any of claims 41-43, wherein the method is repeated to analyze a plurality of samples, e.g., at least 2, 10, 50, 96, 10, 192, or 1000, and
wherein two or more (e.g., all) of the samples are provided using a different process or method of manufacturing, and wherein a process risk score is provided for a plurality of samples (e.g., all samples), or
wherein two or more (e.g., all) of the samples are provided at different time points during a process or method of manufacturing, and wherein a process risk score is provided for a plurality of samples (e.g., all samples).

45. The method of any of claims 41-44, comprising comparing the process risk score of a process or method of manufacturing, e.g., the process or method of manufacturing used to provide the sample, with a reference.

46. The method of any of claims 41-45, comprising comparing the process risk score of a first process or method of manufacturing with a process risk score for a second process or method of manufacturing.

47. The method of any of claims 41-45, comprising comparing the process risk score of a process at a first time point with the process risk score of a process at a second time point.

48. The method of claim 46, comprising, responsive to the comparison, selecting one of the processes or methods of manufacturing, e.g., for further analysis or for further use, e.g., to make the product, e.g., recombinant polypeptide.

49. A database (e.g., memorialized or recorded on a computer readable medium) comprising a library of identifying characteristics for HCPs or protein digestion products and protein risk scores derived from cell culture supernatant of a cell culture (e.g., a CHO, eg., a GS-CHO, cell culture).
